(19) United States
(12) Patent Application Publication
(10) Pub. No.: US 2025/0188491 A1
(43) Pub. Date: Jun. 12, 2025

(54) LENTIVIRAL VECTOR

(71) Applicant: Genespire S.r.l., Milan (IT)

(72) Inventors: Elena Barbon, Cernusco sul Naviglio (Milano) (IT); Alessio Cantore, Milano (IT)

(21) Appl. No.: 18/856,456

(22) PCT Filed: Apr. 14, 2023
(86) PCT No.: PCT/EP2023/059851

§ 371 Date: Oct. 11, 2024

(30) Foreign Application Priority Data
Apr. 14, 2022 (GB) 2205606.3
Apr. 14, 2022 (GB) 2205608.9
Apr. 14, 2022 (GB) 2205615.4
Oct. 11, 2022 (GB) 2214990.0

(51) Int. Cl. C12N 15/86; A61K 48/00; A61P 3/00; C12N 9/90

(52) U.S. Cl. CPC C12N 15/86; A61K 48/0041; A61K 48/0058; A61P 3/00; C12N 9/90; C12N 2740/16043; C12Y 504/99002

(57) ABSTRACT

The present invention relates to lentiviral vectors encoding a methylmalonic acidemia (MMA)-associated polypeptide. The present invention also relates to cells and pharmaceutical compositions comprising said lentiviral vectors and to uses of said lentiviral vectors in treating methylmalonic acidemia (MMA).

### FIELD OF THE INVENTION

The present invention relates to lentiviral vectors encoding a methylmalonic acidemia (MMA)-associated polypeptide. The present invention also relates to cells and pharmaceutical compositions comprising said lentiviral vectors and to uses of said lentiviral vectors in treating methylmalonic acidemia (MMA).

### BACKGROUND TO THE INVENTION

Methylmalonic acidemia (MMA), also called methylmalonic aciduria, is a group of inborn errors of metabolism associated with elevated methylmalonic acid concentration in the blood and urine that may result from the failure to convert methylmalonyl-CoA into succinyl-CoA, during propionyl-CoA metabolism in the mitochondrial matrix.

Isolated MMA (iMMA) may refer to MMA without variations in other metabolites and may be caused by a complete (mut⁰ enzymatic subtype) or partial (mut⁻ enzymatic subtype) deficiency of the enzyme methylmalonyl-CoA mutase; a defect in the transport or synthesis of its cofactor, adenosyl-cobalamin (caused by pathogenic variants in MMAA, MMAB, or MMADHC); or a deficiency of the enzyme methylmalonyl-CoA epimerase (encoded by MCEE) (see e.g. Manoli I, et al. 2005. Isolated Methylmalonic Acidemia. In GeneReviews).

Onset of the manifestations of MMA ranges from the neonatal period to adulthood. Affected children usually exhibit anorexia, failure to thrive, hypotonia, developmental delay, progressive renal failure, functional immune impairment, optic nerve atrophy, and hematologic abnormalities. For example, patients with the most severe form of iMMA, mut⁰, display symptoms such as lethargy, vomiting, respiratory distress, severe ketoacidosis and hyperammonemia in the newborn period. Patients with MMA experience significant morbidity and mortality, and the prognosis for long-term survival is poor (see e.g. Zhou, X., et al., 2018. Intractable & rare diseases research, 7(2), pp. 73-78).

Treatment of MMA typically consists of a protein-restricted diet and in medical management of the symptoms. Nevertheless, patients remain at risk of recurrent episodes of life threatening metabolic crises and multiorgan disease, disability and ultimately death (see e.g. Fraser, J. L. and Venditti, C. P., 2016. Current opinion in pediatrics, 28(6), pp. 682-693). In particular, patients with iMMA often have earlier and more severe clinical manifestations, most of which are coupled with long-term complications involving multiple organs and recurrent metabolic decompensation. Liver transplant is currently the only effective treatment for MMA patients with recurrent metabolic decompensation (see e.g. Jiang, Y. Z. and Sun, L. Y., 2019. Frontiers in Pediatrics, 7, p.87).

Thus, there is a demand for new approaches for treating or preventing MMA, for example iMMA.

A lentiviral vector encoding methylmalonyl-CoA mutase is described in Wong, E. S., et al., 2014. Human gene therapy, 25(6), pp. 529-538. To test the lentiviral vector, Wong et al., used adult Mut^{-/-}MUT^{h2} mice, which have a knockout of mouse MUT but express two copies of a human MUT, thus showing a mild phenotype. Since these mice are transgenic for the human MUT, they are immune tolerant to human MUT, thus possible immune responses triggered by lentiviral gene transfer cannot be properly assessed. Such immune responses are a well-established issue with lentiviral gene therapy (see e.g. VandenDriessche, T., et al., 2007. Journal of Thrombosis and Haemostasis, 5(1), pp. 16-24).

### SUMMARY OF THE INVENTION

The present inventors have developed a gene therapy for treating and/or preventing MMA, for example iMMA. The lentiviral vector mediated gene therapy described herein may allow for a stable gene transfer even in paediatric patients at the first disease stages by virtue of lentiviral vector genomic integration and may therefore alleviate symptoms and limit progressive damage to the hepatocytes.

The present inventors have surprisingly shown that the lentiviral vector mediated gene therapy described herein is safe and efficacious in a model of MMA. The present inventors produced a lentiviral vector encoding a human codon optimized MMUT-coding sequence under the control of a hepatocyte-specific cassette. Administration of the lentiviral vector resulted in a significant decrease of serum methylmalonic acid, indicating improved amino acid metabolism.

Immune-shielded lentiviral vectors may reduce immune responses following administration such that there is no loss of therapeutic effect over time. Targeting transgene expression to hepatocytes (e.g. using one or more miRNA target sequence and/or a hepatocyte-specific promoter) may reduce immune responses in the context of MUT knock out mice, without any expression of the human transgene. In contrast, as described above, Wong et al. administered a lentiviral vector to mice that are transgenic for human MUT, and therefore immune tolerant to expression of the human transgene.

Further, the present inventors have demonstrated that the lentiviral vector of the present invention is efficacious in juveniles. The present inventors showed a stable therapeutic effect (e.g. significant reduction of circulating MMA) up to 12 months post gene therapy following administration to young mice (2 weeks of age), where hepatocyte proliferation is occurring. A stable therapeutic effect in juveniles is not always expected, even with an integrating vector. A stable therapeutic effect may not be obtained if the integrating vector does not transduce those hepatocytes responsible for generating the adult liver following growth and/or if there is counter-selection of corrected hepatocytes. In contrast, Wong et al. treated adult mice.

Therefore, in contrast to Wong et al, the present inventors have demonstrated that the lentiviral vector of the present invention is efficacious in juveniles, in a model that shows a more severe phenotype and that is not immune tolerant to expression of the human transgene.

In one aspect, the present invention provides a lentiviral vector comprising a nucleotide sequence encoding a methylmalonic acidemia (MMA)-associated polypeptide. The lentiviral vector may be an immune-shielded lentiviral vector.

The MMA-associated polypeptide may be selected from methylmalonyl-CoA mutase (MMUT), or a fragment thereof; methylmalonic aciduria type A (MMAA), or a fragment thereof; methylmalonic aciduria type B (MMAB), or a fragment thereof; methylmalonic aciduria and homocystinuria type D (MMADHC), or a fragment thereof; and methylmalonyl-CoA epimerase (MCEE), or a fragment thereof. In preferred embodiments, the MMA-associated polypeptide is MMUT, or a fragment thereof.

Suitably, the MMA-associated polypeptide comprises or consists of an amino acid sequence which is at least 70% identical to one of SEQ ID NOs: 37, 40, 42, 44 or 46, or a fragment thereof. In some embodiments, the MMA-associated polypeptide comprises or consists of an amino acid sequence which is at least 70% identical to SEQ ID NO: 37 or a fragment thereof.

Suitably, the nucleotide sequence encoding a MMA-associated polypeptide comprises or consists of a nucleotide sequence which is at least 70% identical to one of SEQ ID NOs: 38, 39, 41, 43, 45 or 47, or a fragment thereof. In some embodiments, the nucleotide sequence encoding a MMA-associated polypeptide comprises or consists of a nucleotide sequence which is at least 70% identical to one of SEQ ID NOs: 38 or 39, or a fragment thereof. Suitably, the nucleotide sequence encoding a MMA-associated polypeptide is codon-optimised. In some embodiments, the nucleotide sequence encoding a MMA-associated polypeptide comprises or consists of a nucleotide sequence which is at least 70% identical to SEQ ID NO: 39, or a fragment thereof.

The lentiviral vector may be a CD47^{high} lentiviral vector. Suitably, the lentiviral vector is obtained from a CD47^{high} host cell, preferably wherein the host cell is genetically engineered to increase expression of CD47 on the cell surface. Suitably, the lentiviral vector has at least about 2-fold more CD47 on its surface than a lentiviral vector obtained from an unmodified host cell.

The lentiviral vector may be a MHC-I^{free} lentiviral vector. Suitably, the lentiviral vector is obtained from a MHC-I^{free} host cell, preferably wherein the host cell is genetically engineered to disrupt expression of MHC-I on the cell surface. Suitably, the lentiviral vector has less than about 50% of the number of surface-exposed MHC-I molecules that are displayed on a lentiviral vector obtained from an unmodified host cell. Suitably, MHC-I is not detectable on the surface of the lentiviral vector.

The lentiviral vector may be a CD47^{high}/MHC-I^{free} lentiviral vector. Suitably, the lentiviral vector is obtained from a CD47^{high}/MHC-I^{free} host cell. Suitably, the lentiviral vector has at least about 2-fold more CD47 on its surface than a lentiviral vector obtained from an unmodified host cell and MHC-I is not detectable on the surface of the lentiviral vector.

The nucleotide sequence encoding a MMA-associated polypeptide may be operably linked to one or more miRNA target sequences. In some embodiments, the one or more miRNA target sequences suppress transgene expression in one or more cells other than hepatocytes. In some embodiments, the one or more miRNA target sequence suppress transgene expression in hematopoietic-lineage cells and/or antigen-presenting cells.

In some embodiments, the one or more miRNA target sequences are selected from miR-181, miR-142, miR-223, and miR-155 target sequences. In some embodiments, the nucleotide sequence encoding a MMA-associated polypeptide is operably linked to one or more mir-142 target sequence, two or more mir-142 target sequences, three or more mir-142 target sequences, or four or more mir-142 target sequences. In some embodiments, the nucleotide sequence encoding a MMA-associated polypeptide is operably linked to four mir-142 target sequences. In some embodiments, the one or more miRNA target sequences comprise or consist of a nucleotide sequence which is at least 90% identical to SEQ ID NO: 17. In some embodiments, the one or more miRNA target sequences comprise or consist of a nucleotide sequence which is at least 90% identical to SEQ ID NO: 18.

The nucleotide sequence encoding a MMA-associated polypeptide may be operably linked to a liver-specific promoter. In some embodiments, the nucleotide sequence encoding a MMA-associated polypeptide is operably linked to a hepatocyte-specific promoter. Suitably, the nucleotide sequence encoding a MMA-associated polypeptide is operably linked to a transthyretin (TTR) promoter, an alpha-1-antityrpsin (AAT) promoter, a thyroxine-binding globulin (TBG) promoter, an APoE/hAAT promoter, a HCR-hAAT promoter, a LP1 promoter, or a HLP promoter. In some embodiments, the nucleotide sequence encoding a MMA-associated polypeptide is operably linked to a transthyretin (TTR) promoter. In preferred embodiments, the nucleotide sequence encoding a MMA-associated polypeptide is operably linked to an Enh1mTTR (ET) promoter. In some embodiments, the nucleotide sequence encoding a MMA-associated polypeptide is operably linked to a promoter which comprises or consists of a nucleotide sequence which is at least 70% identical to SEQ ID NO: 19.

The lentiviral vector may be pseudotyped. Suitably, the lentiviral vector is VSV.G-pseudotyped. The lentiviral vector may be a self-inactivating (SIN) lentiviral vector. Suitably, the lentiviral vector comprises self-inactivating (SIN) LTRs which comprise or consist of a nucleotide sequence which is at least 70% identical to SEQ ID NO: 23, or a fragment thereof. The lentiviral vector may be an integrating lentiviral vector and/or a replication-defective lentiviral vector. The lentiviral vector may be HIV-derived.

In some embodiments, the lentiviral vector comprises a nucleotide sequence which is at least 70% identical to SEQ ID NO: 36.

In one aspect, the present invention provides a method of producing a lentiviral vector according to the present invention.

In one aspect, the present invention provides a kit or system for producing a lentiviral vector of the present invention.

In one aspect, the present invention provides a cell comprising a lentiviral vector according to the present invention. The cell may be an isolated cell.

In one aspect, the present invention provides a pharmaceutical composition comprising a lentiviral vector or a cell according to the present invention, in combination with a pharmaceutically acceptable carrier, diluent or excipient.

In one aspect, the present invention provides a lentiviral vector, a cell, or a pharmaceutical composition according to the present invention, for use as a medicament.

In one aspect, the present invention provides use of a lentiviral vector, a cell, or a pharmaceutical composition according to the present invention, for the manufacture of a medicament.

In one aspect, the present invention provides a method comprising administering a therapeutically effective amount of a lentiviral vector, a cell, or a pharmaceutical composition according to the present invention, to a subject in need thereof.

In one aspect, the present invention provides a lentiviral vector, a cell, or a pharmaceutical composition according to the present invention, for use in preventing or treating methylmalonic acidemia (MMA), for example isolated MMA (iMMA).

In one aspect, the present invention provides use of a lentiviral vector, a cell, or a pharmaceutical composition according to the present invention, for the manufacture of a medicament for preventing or treating methylmalonic acidemia (MMA), for example isolated MMA (iMMA).

In one aspect, the present invention provides a method of preventing or treating methylmalonic acidemia (MMA), for example isolated MMA (iMMA), comprising administering a therapeutically effective amount of a lentiviral vector, a cell, or a pharmaceutical composition according to the present invention, to a subject in need thereof.

Suitably, the MMA-associated polypeptide is MMUT, or a fragment thereof, and the MMA is mut type MMA (mut-MMA); the MMA-associated polypeptide is MMAA, or a fragment thereof, and the MMA is cblA type MMA (cblA-MMA); the MMA-associated polypeptide is MMAB, or a fragment thereof, and the MMA is cblB type MMA (cblB-MMA); the MMA-associated polypeptide is MMADHC, or a fragment thereof, and the MMA is cblD type MMA (cblD-MMA); or the MMA-associated polypeptide is MCEE, or a fragment thereof, and the MMA is due to MCEE deficiency. In preferred embodiments, the MMA-associated polypeptide is MMUT, or a fragment thereof, and the MMA is mut-MMA.

The lentiviral vector, cell, or pharmaceutical composition may be administered to any subject in need thereof. In some embodiments, the subject is a human subject. In some embodiments, the subject is a juvenile. In some embodiments, the subject is a paediatric patient. In some embodiments, the subject is a neonatal patient or an infantile patient.

The lentiviral vector, cell, or pharmaceutical composition may be administered by any suitable route. In some embodiments, the lentiviral vector, cell, or pharmaceutical composition is administered systemically (e.g. by intravenous injection or intraperitoneal injection). In some embodiments, the lentiviral vector, cell, or pharmaceutical composition is administered locally (e.g. by direct injection, intra-arterial injection, or intraportal injection). In some embodiments, the lentiviral vector, cell, or pharmaceutical composition is administered locally to the liver (e.g. by intrahepatic injection, intrahepatic arterial injection, or intraportal injection).

The lentiviral vector, cell, or pharmaceutical composition may be administered in any suitable dose. In some embodiments, the lentiviral vector is administered at a dose of at least about 10⁸ TU/kg, at least about 10⁹ TU/kg, or at least about 10¹⁰ TU/kg. In some embodiments, the lentiviral vector is administered in a dose of from about 10⁸ to about 10¹¹ TU/kg, from about 10⁸ to about 10¹⁰ TU/kg, or from about 10⁹ to about 10¹⁰ TU/kg.

The lentiviral vector may integrate into the genome of liver cells and be maintained as the liver cells duplicate. Suitably, the lentiviral vector integrates into the genome of hepatocytes and is maintained as the hepatocytes duplicate.

Following administration, the subject's plasma methylmalonic acid levels and/or urine methylmalonic acid levels may be reduced and/or normalised; and/or the subject's mitochondrial function may be improved.

In one aspect, the present invention provides an immune-shielded lentiviral vector for use in a method of therapy. In one aspect, the present invention provides a cell for use in a method of therapy, wherein the cell comprises an immune-shielded lentiviral vector.

In one aspect, the present invention provides use of an immune-shielded lentiviral vector for the manufacture of a medicament for treatment or prevention of a disease. In one aspect, the present invention provides use of a cell for the manufacture of a medicament for treatment or prevention of a disease, wherein the cell comprises an immune-shielded lentiviral vector.

In one aspect, the present invention provides a method of treatment, wherein the method comprises administration of an immune-shielded lentiviral vector to a subject. In one aspect, the present invention provides a method of treatment, wherein the method comprises administration of a cell to a subject, wherein the cell comprises an immune-shielded lentiviral vector.

In one aspect, the present invention provides an immune-shielded lentiviral vector for use in a method of therapy, wherein the method comprises administration of the immune-shielded lentiviral vector to a juvenile or paediatric subject. In one aspect, the present invention provides a cell for use in a method of therapy, wherein the cell comprises an immune-shielded lentiviral vector, and wherein the method comprises administration of the cell to a juvenile or paediatric subject.

In one aspect, the present invention provides use of an immune-shielded lentiviral vector for the manufacture of a medicament for treatment or prevention of a disease, wherein the treatment or prevention comprises administration of the immune-shielded lentiviral vector to a juvenile or paediatric subject. In one aspect, the present invention provides use of a cell for the manufacture of a medicament for treatment or prevention of a disease, wherein the cell comprises an immune-shielded lentiviral vector, and wherein the treatment or prevention comprises administration of the cell to a juvenile or paediatric subject.

In one aspect, the present invention provides a method of treatment, wherein the method comprises administration of an immune-shielded lentiviral vector to a juvenile or paediatric subject. In one aspect, the present invention provides a method of treatment, wherein the method comprises administration of a cell to a juvenile or paediatric subject, wherein the cell comprises an immune-shielded lentiviral vector.

In one aspect, the present invention provides an immune-shielded lentiviral vector for use in a method of treatment of a juvenile or paediatric subject. In one aspect, the present invention provides a cell for use in a method of treatment of a juvenile or paediatric subject, wherein the cell comprises an immune-shielded lentiviral vector.

In one aspect, the present invention provides use of an immune-shielded lentiviral vector for the manufacture of a medicament for treatment or prevention of a disease in a juvenile or paediatric subject. In one aspect, the present invention provides use of a cell for the manufacture of a medicament for treatment or prevention of a disease in a juvenile or paediatric subject, wherein the cell comprises an immune-shielded lentiviral vector.

In one aspect, the present invention provides a method of treatment of a juvenile or paediatric subject, wherein the method comprises administration of an immune-shielded lentiviral vector to the juvenile or paediatric subject in need thereof. In one aspect, the present invention provides a method of treatment of a juvenile or paediatric subject, wherein the method comprises administration of a cell to the juvenile or paediatric subject in need thereof, wherein the cell comprises an immune-shielded lentiviral vector.

In some embodiments, the subject is a neonatal subject or an infantile subject.

In some embodiments, the method comprises transduction of a liver cell with the immune-shielded lentiviral vector. In some embodiments, the method comprises transduction of a hepatocyte with the immune-shielded lentiviral vector.

In some embodiments, the therapy, treatment or prevention is treatment or prevention of methylmalonic acidemia (MMA).

In some embodiments, the immune-shielded lentiviral vector comprises a nucleotide sequence encoding a methylmalonic acidemia (MMA)-associated polypeptide.

### DESCRIPTION OF DRAWINGS

FIG. 1—Design and testing of a lentiviral vector (LV) encoding a MUT transgene
(a) Schematic of the third-generation lentiviral vectors (provirus). LTRs, long terminal repeats: the 5′ LTR and the 3′ LTR each have an almost complete deletion of the U3 region (ΔU3); ψ, the psi packaging sequence; RRE, Rev response element; cPPT, central polypurine tract; ET, enhanced transtyretin promoter; MUT wt/co, MUT coding sequences (wild-type or codon optimised); WPRE, posttranscriptional element from the genome of the woodchuck hepatitis virus; 4×mir142T, four copies of miR-142 target sequence in tandem. (b) Western blot analysis of MUT in Huh7 cells transduced with LV-ET.MUTwt or LV-ET.MUTco. kDa, molecular weight marker; Ctrl, untransduced cells. (c) Western blot analysis of MUT in Huh7 cells with LV-hAAT.MUTwt or LV-hAAT.MUTco. kDa, molecular weight marker. (d) MUT protein expression as determined by WB densitometry analysis and normalized on vector copy number (VCN) in Huh7 transduced with LV-ET.MUTwt/co at different MOI. (e) MUT protein expression as determined by WB densitometry analysis and normalized on vector copy number (VCN) in Huh7 transduced with LV-hAAT.MUTwt/co at different MOI. (f) MUT protein expression as determined by WB densitometry analysis and normalized on GAPDH housekeeping gene in Huh7 cells transduced with LV-ET.BSEPco_1 or LV-hAAT.BSEPco_1.

FIG. 2—In vivo gene transfer with LV-ET.MUT
(a) Vector copy number per cell (VCN/cell) in the liver of C57Bl/6 mice treated with LV-ET.MUTwt or LV-ET.MUTco. (b) MUT mRNA, measured as WPRE normalized on HPRT expression, in mice treated with LV-ET.MUTwt or LV-ET.MUTco. (c) Western blot analysis of MUT in liver samples from C57Bl/6 mice treated with LV-ET.MUTwt or LV-ET.MUTco. (d) MUT protein expression as determined by WB densitometry analysis and normalized on beta-actin housekeeping gene in Huh7 cells transduced with LV-ET.MUTco or LV-hAAT.MUTco. UT, untreated mice.

FIG. 3—Evaluation of long-term LV gene therapy in MCK-MUT −/− mice
(a) Percent MCK-Mut survival over time. [Mantel-Cox test p=0.0033]. (b) Measurement of MCK-Mut weight (grams) over time. **** p<000.1 by two-way ANOVA comparing MCK-Mut−/− +LV with MCK-Mut−/− group. (c) Measurement of blood methylmalonic acid (MMA) in MCK-Mut mice over time. Student t test on data 50 weeks post LV, p=0.00029 MCK-Mut−/−+LV vs MCK-Mut −/−. (d) Measurement of serum FGF-21 biomarker. Multiple comparisons of the experimental groups were performed by One-way ANOVA. MCK-Mut −/−, untreated KO; MCK-Mut −/−+LV, KO animals treated with LV-ET.MUTwt; MCK-Mut+/−, heterozygous control animals (phenotypically normal).

FIG. 4—Vector copy number and MUT protein expression in the liver MCK-Mut −/− treated mice 12 months post LV administration
(a) Vector copy number per cell (VCN/cell) assessed in the liver (b) MUT mRNA, measured as WPRE normalized on HPRT expression, and (c) Western blot analysis of MUT in liver samples from MCK-Mut mice 12 months post LV administration. KO+LV, KO animals treated with LV-ET.MUT; WT, phenotypically normal animals, KO, untreated KO (MCK-Mut −/−).

FIG. 5—Liver transduction efficiency and histopathology in MCK-Mut −/− treated mice 6 months post LV administration (a) RNA in situ hybridization performed on liver tissue section, with a probe against the WPRE element. (b) Image quantification of liver transduced area in MCK-Mut −/− mice treated with LV-ET.MUT. (c) Liver histopathology images. KO+LV, KO animals treated with LV-ET.MUT; WT, phenotypically normal animals, KO, untreated KO (MCK-Mut −/−).

FIG. 6—Amelioration of the liver mitochondriopathy of MCK-Mut −/− mice
(a) Example electron microscopy images of liver samples. (b-d) Image quantification of (b) mitochondrial area, (c) mitochondrial perimeter and (d) mitochondrial shape, measured as circularity index [2-way ANOVA, p<0.0001****]. (e) Quantification of methylmalonic acid in liver tissue (liver MMA). [One-way ANOVA, p<0.0001****]. KO+LV, KO animals treated with LV-ET.MUT; WT, phenotypically normal animals, KO, untreated KO (MCK-Mut −/−).

FIG. 7—Amelioration of the kidney mitochondriopathy of MCK-Mut −/− mice
(a) Example electron microscopy images of kidney samples. (b) Image quantification of ring-shaped structures [2-way ANOVA, p<0.0001****]. (c) Quantification of methylmalonic acid in kidney tissue (kidney MMA). [Kruskal-Wallis multiple comparison test, p=0.04*]. KO UT, untreated KO (MCK-Mut −/−); KO+LV, KO animals treated with LV-ET.MUT; WT, phenotypically normal animals.

FIG. 8—Reduction of brain MMA in MCK-Mut −/− treated mice
(a) Example electron microscopy images of brain samples. Quantification of methylmalonic acid in the brain (brain MMA). [Kruskal-Wallis multiple comparison test, p=0.02*]. KO UT, untreated KO (MCK-Mut −/−); KO+LV, KO animals treated with LV-ET.MUT; WT, phenotypically normal animals.

FIG. 9—In vivo gene therapy with CD47^{high}/MHC^{free} LV.MUTwt
(a) Measurement of MCK-Mut weight (grams) over time. (b) Measurement of blood methylmalonic acid (MMA) in MCK-Mut mice over time. KO UT, untreated KO (MCK-Mut −/−); KO+LV, KO animals treated with CD47^{high}/MHC-I^{free} LV.MUTwt; WT, phenotypically normal animals.

FIG. 10—Comparison of in vivo gene therapy with LV.MUTwt and LV.MUTco at matched LV dose of 2.5E10 TU/kg
(a) Measurement of MCK-Mut weight (grams) over time. (b) Measurement of blood methylmalonic acid (MMA) in MCK-Mut mice over time. KO UT, untreated KO (MCK-Mut −/−); KO+LV.MUT, KO animals treated with LV-ET.MUTwt; KO+LV.MUTco, KO animals treated with LV-ET.MUTco; WT, phenotypically normal animals.

FIG. 11—Design and testing of a lentiviral vector (LV) encoding a BSEP transgene
(a) Schematic of the third-generation lentiviral vectors (provirus). LTRs, long terminal repeats: the 5′ LTR and the 3′ LTR each have an almost complete deletion of the U3 region (ΔU3); ψ, the psi packaging sequence; RRE, Rev response element; cPPT, central polypurine tract; ET, enhanced transtyretin promoter; BSEP wt/co, BSEP coding sequences (wild-type or codon optimised); WPRE, posttranscriptional element from the genome of the woodchuck hepatitis virus; 4×mir142T, four copies of miR-142 target sequence in tandem. (b) Flow cytometry analysis on Huh7 cells transduced with LV-ET.BSEPwt, LV-ET.BSEPco_1 or LV-ET.BSEPco_2 at different MOI. UT, untransduced Huh7 cells. MFI, mean fluorescence intensity. (c) Histogram reporting the vector copy number (VCN) per cell (VCN/cell) in Huh7 transduced with LV-ET.BSEPwt, LV-ET.BSEPco_1 or LV-ET.BSEPco_2 at different MOI. (d) Histogram reporting the vector copy number (VCN) per cell (VCN/cell) in Huh7 transduced with LV-ET.BSEPco_1 or LV-hAAT.BSEPco_1 at different MOI. (e) Western Blot analysis of BSEP protein expression on Huh7 cell lysate. UT, untransduced Huh7; hAAT, cells transduced with LV-hAAT.BSEPco; ET, cells transduced with LV-ET.BSEPco. (f) BSEP protein expression as determined by WB densitometry analysis and normalized on GAPDH housekeeping gene in Huh7 cells transduced with LV-ET.BSEPco or LV-hAAT.BSEPco.

FIG. 12—Immunofluorescence analysis of LV-ET.BSEPco_1 in transduced Huh7 cells
Left upper panel, Huh7 cells transduced with LV-ET.BSEPco_1 and stained with Hoechst. Right upper panel, Huh7 cells untransduced (Cntrl-) and stained with Hoechst. Left lower panel, Huh7 cells transduced with LV-ET.BSEPco_1 and stained with an anti-BSEP antibody. Right lower panel, Huh7 cells untransduced (Cntrl-) and stained with an anti-BSEP antibody.

FIG. 13—Evaluation of LV gene therapy in Abcb11−/− mice
(a) Serum bile acids in Abcb11−/− animals treated with LV-ET.BSEPco (KO+LV) compared to Abcb11−/− untreated animals (KO UT) and to WT untreated animals (WT UT). (b) Serum total bilirubin in Abcb11−/− animals treated with LV-ET.BSEPco (KO+LV) compared to Abcb11−/− untreated animals (KO UT). Dotted line indicates the upper limit of the bilirubin levels (mg/dL) measured in WT control mice. (c) Serum alkaline phosphatase (ALP) levels (IU/L) in Abcb11−/− animals treated with LV-ET.BSEPco (KO+LV) compared to Abcb11−/− untreated animals (KO UT) and to WT untreated animals (WT UT). Dotted line indicates the upper limit of the ALP levels measured in a cohort of WT control mice.

FIG. 14—Design and testing of a lentiviral vector (LV) encoding a BSEP transgene
(a) Schematic of the third-generation lentiviral vectors (provirus). LTRs, long terminal repeats: the 5′ LTR and the 3′ LTR each have an almost complete deletion of the U3 region (ΔU3); ψ, the psi packaging sequence; RRE, Rev response element; cPPT, central polypurine tract; ET, enhanced transtyretin promoter; DBT wt/co, DBT coding sequences (wild-type or codon optimised); WPRE, posttranscriptional element from the genome of the woodchuck hepatitis virus; 4×mir142T, four copies of miR-142 target sequence in tandem. (b) Western blot analysis of DBT in Huh7 cells transduced with LV-ET-DBTwt or LV-ET-DBTco. kDa, molecular weight marker; Ctrl, untransduced cells. (c) Western blot analysis of DBT in Huh7 cells transduced with LV-hAAT-DBTwt or LV-hAAT-DBTco. kDa, molecular weight marker; Ctrl, untransduced cells. (d) DBT protein expression as determined by WB densitometry analysis and normalized on vector copy number (VCN) in Huh7 transduced with LV-ET.DBTwt/co at different MOI. (e) VCN in Huh7 transduced with LV-hAAT.DBTwt/co at different MOI. (f) DBT protein expression as determined by WB densitometry analysis and normalized on GAPDH housekeeping gene in Huh7 cells transduced with LV-ET.DBTwt/co or LV-hAAT.DBTwt/co.

FIG. 15—In vivo gene transfer with LV-ET.DBT
(a) Vector copy number (VCN) in liver of C57Bl/6 WT mice administered with LV-ET.DBTwt or LV-ET.DBTco LV. (b) DBT mRNA, normalized on HPRT expression, in mice treated with LV-ET.DBTwt or LV-ET.DBTco. UT, untreated mice. (c) Western blot analysis of DBT in liver samples from C57Bl/6 mice treated with LV-ET.DBTwt or LV-ET.DBTco. (d) DBT protein expression as determined by WB densitometry analysis and normalized on β-actin housekeeping gene.

FIG. 16—Evaluation of LV gene therapy in iMSUD mice
(a) Survival of iMSUD mice treated with LV-ET.DBTco (KO+LV) compared to iMSUD untreated (KO UT) and WT control littermates (WT). ****p<0.0001, Long-rank test. (b) Branched chain amino-acids levels normalized on alanine (BCAA/Ala) in iMSUD mice treated with LV-ET.DBTco (KO+LV) compared to iMSUD untreated (KO UT) and WT control littermates (WT). (c) Alloisoleucine (μM) in iMSUD mice treated with LV-ET.DBTco (KO+LV) compared to iMSUD untreated (KO UT) and WT control littermates (WT).

### DETAILED DESCRIPTION

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The publications discussed herein are provided solely for their disclosure prior to the filing date of the present application. Nothing herein is to be construed as an admission that such publications constitute prior art to the claims appended hereto.

The skilled person will understand that they can combine all features of the invention disclosed herein without departing from the scope of the invention as disclosed.

### Lentiviral Vector

In one aspect, the present invention provides a lentiviral vector comprising a protein-coding sequence, wherein the protein-coding sequence is a nucleotide sequence encoding a methylmalonic acidemia (MMA)-associated polypeptide.

A “lentiviral vector” may refer to an enveloped lentiviral genome (i.e. a lentiviral particle). For example, the pharmaceutical composition of the present invention preferably comprises the lentiviral vector in the form of a lentiviral particle and the lentiviral vector is preferably administered in the form of a lentiviral particle. In other embodiments, a “lentiviral vector” may comprise a lentiviral genome, optionally wherein the lentiviral genome is enveloped. As used herein, a “lentiviral genome” may refer to a genome that comprises at least one element derived or derivable from a lentivirus genome. Suitably, a lentiviral genome comprises at least one element that is involved in the mechanisms by which a lentivirus infects cells, expresses genes, and/or is replicated.

Lentivirus is a genus of retroviruses, which contain an RNA genome that is converted to DNA in the transduced cell by a virally encoded reverse transcriptase. Lentiviral vectors can transduce a wide range of cell types and integrate into the host genome in both dividing and post-mitotic cells, resulting in long-term expression of the protein-coding sequence both in vitro and in vivo (see e.g. Tiscornia, G., et al., 2006. Nature protocols, 1(1), pp. 241-245).

The basic genes required for lentivirus survival and function are the gag, pol, and env genes: gag encodes structural proteins; pol encodes enzymes required for reverse transcription and integration into the host cell genome; and env encodes the viral envelope glycoprotein (see e.g. Milone, M. C. and O'Doherty, U., 2018. Leukemia, 32(7), pp. 1529-1541). Lentiviruses may also have additional cis-acting elements, such as a rev response element (RRE), which enables the efficient export of RNA transcripts of the integrated provirus from the nucleus to the cytoplasm of an infected target cell; a retroviral psi packaging element, which is involved in regulating the essential process of packaging the retroviral RNA genome into the viral capsid during replication; a primer binding site (PBS), where reverse transcription is initiated; the TAT activation region (TAR); splice donor and acceptor sites; and central and terminal polypurine tracts, which allow initiation of plus-strand synthesis.

In a lentivirus genome, these elements are typically flanked at both ends by regions called long terminal repeats (LTRs). The LTRs are responsible for integration and transcription. LTRs may also serve as enhancer-promoter sequences and can control the expression of the lentiviral genes. The LTRs themselves are identical or near-identical sequences that can typically be divided into three regions: U3, R and U5. LTRs may be naturally occurring or may be modified. For example, U3 and U5 modifications are described in Iwakuma, T., et al., 1999. Virology, 261(1), pp. 120-132.

The lentiviral vector of the present invention may comprise a minimal lentiviral genome. As used herein, a “minimal lentiviral genome” may mean that the lentiviral genome has been manipulated so as to remove the non-essential elements and to retain the essential elements in order to provide the required functionality to infect, transduce and deliver a nucleotide sequence of interest to a target host cell (see e.g. Kim, V. N., et al., 1998. Journal of virology, 72(1), pp. 811-816 and Sertkaya, H., et al., 2021. Scientific reports, 11(1), pp. 1-15).

A lentiviral genome may comprise from 5′ to 3′: a 5′ LTR, one or more lentiviral-derived cis-acting elements, and a 3′ LTR. Suitably, a lentiviral genome may comprise from 5′ to 3′: a 5′ LTR, a RRE, and a 3′ LTR. Suitably, a lentiviral genome may comprise from 5′ to 3′: a 5′ LTR, a retroviral psi packaging element, a RRE, and a 3′ LTR. Suitably, a lentiviral genome may comprise from 5′ to 3′: a 5′ LTR, a retroviral psi packaging element, a RRE, a cPPT, and a 3′ LTR. Suitably, a lentiviral genome may comprise from 5′ to 3′: a 5′ LTR, a PBS, a retroviral psi packaging element, a RRE, a cPPT, and a 3′ LTR.

A lentiviral genome may further comprise a protein-coding sequence and, optionally, one or more regulatory elements (e.g. operably linked to the protein-coding sequence). Suitably, a lentiviral genome may comprise from 5′ to 3′: a 5′ LTR, a RRE, a protein-coding sequence, and a 3′ LTR. Suitably, a lentiviral genome may comprise from 5′ to 3′: a 5′ LTR, a retroviral psi packaging element, a RRE, a protein-coding sequence, and a 3′ LTR. Suitably, a lentiviral genome may comprise from 5′ to 3′: a 5′ LTR, a retroviral psi packaging element, a RRE, a cPPT, a protein-coding sequence, and a 3′ LTR. Suitably, lentiviral genome may comprise from 5′ to 3′: a 5′ LTR, a PBS, a retroviral psi packaging element, a RRE, a cPPT, a protein-coding sequence, and a 3′ LTR.

The lentiviral vector of the present invention may be replication-defective. Typically, at least part of one or more protein coding regions essential for replication may be removed from the lentiviral genome. This makes the lentiviral vector “replication-defective” or “replication-incompetent”. Suitably, one or more of gag, pol, rev, and env genes are deleted (at least partially) in a replication-defective lentiviral vector. Suitably, each of the gag, pol, rev, and env genes are deleted (at least partially) in a replication-defective lentiviral vector. Optionally, the lentiviral vector lacks a functional gag-pol and/or env gene and/or other genes essential for replication.

The lentiviral vector of the present invention may be derived from any lentivirus. As used herein “lentivirus-derived” or “lentivirus-based” may mean that the lentiviral genome comprises one or more elements from said lentivirus. For example, the coding regions of viral proteins may be deleted, but one or more cis-acting element may be retained from said lentivirus.

The lentiviral vector may be derived from a primate lentivirus. Examples of “primate” lentiviruses include, but are not limited to, human immunodeficiency virus (HIV) and simian immunodeficiency virus (SIV). The lentiviral vector may be derived from a non-primate lentivirus (i.e. derived from a lentivirus which does not primarily infect primates, especially humans). Examples of “non-primate” lentiviruses include, but are not limited to, the prototype “slow virus” visna/maedi virus (VMV), caprine arthritis-encephalitis virus (CAEV), equine infectious anaemia virus (EIAV), feline immunodeficiency virus (FIV), and bovine immunodeficiency virus (BIV).

Suitably, the lentiviral vector of the present invention is a HIV-derived lentiviral vector. As used herein “HIV-derived” or “HIV-based” may mean that the lentiviral genome comprises one or more element from HIV. For example, the coding regions of HIV viral proteins may be deleted, and one or more HIV cis-acting element may retained in the lentiviral genome (see e.g. Johnson, N. M., et al., 2021. Molecular Therapy-Methods & Clinical Development, 21, pp. 451-465). A HIV-derived lentiviral genome may comprise from 5′ to 3′: a 5′ LTR, one or more HIV-derived cis-acting elements (e.g. RRE and/or cPPT), and a 3′ LTR.

Suitably, the lentiviral vector of the present invention is a HIV-1-derived lentiviral vector. The prototype lentiviral vector system is based on HIV-1 (see e.g. Merten, O. W., et al., 2016. Molecular Therapy-Methods & Clinical Development, 3, p.16017). It has been shown that sequences that extend into the gag open reading frame may be important for packaging of HIV-1. Therefore, HIV-1 vectors often contain the relevant portion of gag in which the translational initiation codon has been mutated. In addition, HIV-1 vectors often also contain a portion of the env gene that includes the RRE. Rev binds to RRE, which permits the transport of full-length or singly spliced mRNAs from the nucleus to the cytoplasm. In the absence of rev and/or a RRE, full-length HIV-1 RNAs may accumulate in the nucleus. Alternatively, a constitutive transport element from certain simple retroviruses such as Mason-Pfizer monkey virus can be used to relieve the requirement for rev and a RRE. A HIV-1-derived lentiviral genome may comprise from 5′ to 3′: a 5′ LTR, one or more HIV-1-derived cis-acting elements (e.g. a PBS, a retroviral psi packaging element, a RRE and/or a cPPT), and a 3′ LTR.

The lentiviral vector of the present invention may be a self-inactivating lentiviral vector. As used herein, “self-inactivating” or “SIN” lentiviral vectors may comprise lentiviral genomes in which the lentiviral enhancer and promoter sequences have been deleted (see e.g. Zufferey, R., et al., 1998. Journal of virology, 72(12), pp. 9873-9880 and Miyoshi, H., et al., 1998. Journal of virology, 72(10), pp. 8150-8157). SIN lentiviral vectors can be generated and transduce non-dividing cells in vivo with an efficacy similar to that of wild-type vectors. The transcriptional inactivation of the long terminal repeat (LTR) in the SIN provirus can prevent mobilisation by replication-competent virus. This can also enable the regulated expression of genes from internal promoters by eliminating any cis-acting effects of the LTR.

The lentiviral vector of the present invention may be integration competent. As used herein, an “integration competent” lentiviral vector is capable of integrating into the genome of a host cell. In contrast to integration competent lentiviral vectors, integration defective lentiviral vectors can be produced, for example, either by packaging the lentiviral vector with catalytically inactive integrase (such as an HIV integrase bearing the D64V mutation in the catalytic site) or by modifying or deleting essential att sequences from the lentiviral genome LTR, or by a combination of the above (see e.g. Wanisch, K. and Yenez-Munoz, R. J., 2009. Molecular Therapy, 17(8), pp. 1316-1332).

The lentiviral vector of the present invention may be replication-defective and integrating. The lentiviral vector of the present invention may be replication-defective, integrating, and self-inactivating. The lentiviral vector of the present invention may be replication-defective, integrating, self-inactivating, and HIV-derived.

The lentiviral vector of the present invention may be a lentiviral particle. A “lentiviral particle” may refer to an enveloped lentiviral genome. Lentiviral particles may be generated by co-transfection of a plasmid containing a lentiviral genome (e.g. a “transfer vector”) with helper plasmids (e.g. “packaging vectors” encoding gag-pol and/or rev and “envelope vectors” encoding env) into host cells and harvesting of the lentivirus-containing supernatant afterwards.

The lentiviral vector of the present invention may be pseudotyped. Pseudotyping lentiviral vectors with naturally occurring or engineered lentiviral envelopes can allow targeted transduction of specific cell types (see e.g. Joglekar, A. V. and Sandoval, S., 2017. Human Gene Therapy Methods, 28(6), pp. 291-301). Suitably, the lentiviral vector is pseudotyped to allow transduction of liver cells (e.g. hepatocytes).

The lentiviral vector of the present invention may be VSV-G pseudotyped. Vesicular stomatitis virus G protein (VSV-G) is a commonly used envelope protein for pseudotyping. VSV-G is a trimeric protein that binds phosphatidylserine and low-density lipoprotein receptors on a cell surface to endocytose into the cell. VSV-G pseudotyped lentiviral vectors may efficiently transduce liver cells (e.g. hepatocytes).

The lentiviral vector of the present invention may be replication-defective, integrating, and VSV-G pseudotyped. The lentiviral vector of the present invention may be replication-defective, integrating, self-inactivating, and VSV-G pseudotyped. The lentiviral vector of the present invention may be replication-defective, integrating self-inactivating, HIV-derived, and VSV-G pseudotyped.

### Immune-Shielded Lentiviral Vector

The lentiviral vector of the present invention may be an immune-shielded lentiviral vector.

As used herein, an “immune shielded lentiviral vector” may refer to a lentiviral vector which is modified to reduce immune responses following administration. For example, the immune-shielding may reduce activation of acute inflammatory response after administration.

In some embodiments, the lentiviral vector of the present invention is: (i) a CD47^{high} lentiviral vector; (ii) a MHC-I^{free} lentiviral vector; and/or (iii) comprises one or more miRNA target sequences (e.g. which suppress transgene expression in antigen-presenting cells). In preferred embodiments, the lentiviral vector of the present invention is a CD47^{high}/MHC-I^{free} lentiviral vector and comprises one or more miRNA target sequences (e.g. which suppress transgene expression in antigen-presenting cells). Each of these modifications may act to reduce immune responses following administration.

An immune-shielded lentiviral vector may be “phagocytosis-shielded” to reduce uptake by professional phagocytes. In some embodiments, the lentiviral vector is a phagocytosis-shielded lentiviral vector. For example, the lentiviral vector may be a CD47^{high} lentiviral vector.

### CD47^{high} Lentiviral Vectors

The lentiviral vector of the present invention may be a CD47^{high} lentiviral vector. As used herein, a “CD47^{high} lentiviral vector” may refer to a lentiviral vector with increased levels of CD47 (or a fragment thereof) on its surface. A CD47^{high} lentiviral vector may have reduced uptake by professional phagocytes.

CD47 (Cluster of Differentiation 47) also known as integrin associated protein (IAP) is a transmembrane protein that in humans is encoded by the CD47 gene. Phagocytosis is physiologically inhibited by CD47, which is a ubiquitously expressed ligand of signal regulatory protein α (SIRP-α) receptor, that is expressed by professional phagocytes. CD47 may be incorporated into lentiviral vectors when they bud from producer cells.

The lentiviral vector of the present invention may comprise one or more CD47 polypeptides (or a fragment thereof) on its surface. The amount of CD47 (or a fragment thereof) on the surface may be enough to reduce uptake by professional phagocytes. Any suitable assay to quantify the amount of CD47 polypeptides (or fragments thereof) present on the surface of the lentiviral vector may be used.

In some embodiments, the density of CD47 polypeptides (or fragments thereof) may be determined by immunostaining for CD47 and total internal reflection fluorescence microscopy, e.g. as described in US2010/0316570A1. The CD47 polypeptides (or fragments thereof) may be present in a density of at least about 20 molecules/μm², at least about 25 molecules/μm², at least about 30 molecules/μm², at least about 35 molecules/μm², at least about 40 molecules/μm², at least about 45 molecules/μm², at least about 50 molecules/μm², at least about 60 molecules/μm², at least about 70 molecules/μm², at least about 80 molecules/μm², at least about 90 molecules/μm², at least about 100 molecules/μm², at least about 150 molecules/μm², at least about 200 molecules/μm², at least about 250 molecules/μm², at least about 300 molecules/μm², at least about 350 molecules/μm², at least about 400 molecules/μm², at least about 450 molecules/μm², at least about 500 molecules/μm², at least about 600 molecules/μm², at least about 700 molecules/μm², at least about 800 molecules/μm², at least about 900 molecules/μm², or at least about 1000 molecules/μm². The CD47 polypeptides (or fragments thereof) may be present in a density of about 1000 molecules/μm² or less, about 500 molecules/μm² or less, or about 250 molecules/μm² or less.

The CD47 polypeptides (or fragments thereof) may be present in a density of from about 20 molecules/μm² to about 1000 molecules/μm², from about 20 molecules/μm² to about 500 molecules/μm², or from about 20 molecules/μm² to about 250 molecules/μm².

In some embodiments, the amount of CD47 polypeptides (or fragments thereof) may be determined by immunostaining for CD47 and electron microscopy, as described in Milani, M., et al., 2019. Science Translational Medicine, 11(493), p.eaav7325. The CD47 polypeptides (or fragments thereof) may be detected in an amount of at least about 10 gold particles/lentiviral particle, at least about 15 gold particles/lentiviral particle, or at least about 20 gold particles/lentiviral particle. The CD47 polypeptides (or fragments thereof) may be detected in an amount of about 100 gold particles/lentiviral particle or less, about 80 gold particles/lentiviral particle or less, or about 60 gold particles/lentiviral particle or less. The CD47 polypeptides (or fragments thereof) may be detected in an amount of from about 10 to about 100 gold particles/lentiviral particle, from about 15 to about 80 gold particles/lentiviral particle, or from about 20 to about 60 gold particles/lentiviral particle.

The lentiviral vector of the present invention may be obtained from a CD47^{high} host cell. As used herein, a “CD47^{high} host cell” may refer to a host cell with increased levels of CD47 (or a fragment thereof) on its surface.

A CD47^{high} host cell may be genetically engineered to increase expression of CD47 (or a fragment thereof) on the cell surface. For example, the host cell may comprise a vector encoding CD47 (or a fragment thereof) or may be edited to introduce a nucleotide sequence encoding CD47 (or a fragment thereof) into its genome. Suitably, the host cell is transduced with a viral vector encoding a CD47 polypeptide (or a fragment thereof).

A CD47^{high} host cell may have a higher concentration of CD47 (or a fragment thereof) on its surface than an unmodified host cell (e.g. an unmodified producer cell or packaging cell, as described herein). Suitably, the host cell has at least about 2-fold, at least about 3-fold, at least about 4-fold, at least about 5-fold, at least about 10-fold, at least about 20-fold, or at least about 30-fold more CD47 on its cell surface than an unmodified host cell. Suitably, the host cell has from about 5-fold to about 30-fold more CD47 (or a fragment thereof) on its cell surface than an unmodified host cell.

Suitably, the lentiviral vector of the present has a higher concentration of CD47 (or a fragment thereof) on its surface than a lentiviral vector obtained from an unmodified host cell (e.g. an unmodified producer cell or packaging cell, as described herein). Suitably, the lentiviral vector has at least about 2-fold, at least about 3-fold, at least about 4-fold, at least about 5-fold, at least about 10-fold, at least about 20-fold, at least about 30-fold, at least about 40-fold, or at least about 50-fold more CD47 (or a fragment thereof) on its surface than a lentiviral vector obtained from an unmodified host cell. Suitably, the lentiviral vector has from about 5-fold to about 30-fold more CD47 (or a fragment thereof) on its surface than a lentiviral vector obtained from an unmodified host cell.

CD47 is a member of the immunoglobulin (Ig) superfamily of membrane proteins, with a single IgV-like domain at its N-terminus, a highly hydrophobic stretch with five membrane-spanning segments and an alternatively spliced cytoplasmic C-terminus ranging in length from 3 to 36 amino acids. Mouse, rat, bovine and human CD47 molecules have been cloned and show about 70% overall amino acid identity (see e.g. Brown, E. J. and Frazier, W. A., 2001. Trends in cell biology, 11(3), pp. 130-135).

The CD47 polypeptide (or a fragment thereof) may be a human CD47 polypeptide (or a fragment thereof). A CD47 polypeptide may have an amino acid sequence of UniProtKB Q08722.

Exemplary CD47 polypeptides are provided by SEQ ID NOs: 1-4. Suitably, a CD47 polypeptide comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to any of SEQ ID NOs: 1-4. Suitably, a CD47 polypeptide comprises or consists of the amino acid sequence of any of SEQ ID NOs: 1-4.

Exemplary CD47 polypeptide
(SEQ ID NO: 1)
MWPLVAALLLGSACCGSAQLLENKTKSVEFTFCNDTVVIPCFVTNMEAQNTTEVYVKWKFKG
RDIYTFDGALNKSTVPTDESSAKIEVSQLLKGDASLKMDKSDAVSHTGNYTCEVTELTREGE
TIIELKYRVVSWESPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSEVIAILVIQVIA
YILAVVGLSLCIAACIPMHGPLLISGLSILALAQLLGLVYMKFVASNQKTIQPPRNN
Exemplary CD47 polypeptide
(SEQ ID NO: 2)
MWPLVAALLLGSACCGSAQLLENKTKSVEFTFCNDTVVIPCFVTNMEAQNTTEVYVKWKFKG
RDIYTFDGALNKSTVPTDESSAKIEVSQLLKGDASLKMDKSDAVSHTGNYTCEVTELTREGE
TIIELKYRVVSWESPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKFVASNQKTIQPPRKAVEEPL
NAFKESKGMMNDE
Exemplary CD47 polypeptide
(SEQ ID NO: 3)
MWPLVAALLLGSACCGSAQLLENKTKSVEFTFCNDTVVIPCFVTNMEAQNTTEVYVKWKFKG
RDIYTFDGALNKSTVPTDESSAKIEVSQLLKGDASLKMDKSDAVSHTGNYTCEVTELTREGE
TIIELKYRVVSWESPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKFV
Exemplary CD47 polypeptide
(SEQ ID NO: 4)
MWPLVAALLLGSACCGSAQLLENKTKSVEFTFCNDTVVIPCFVTNMEAQNTTEVYVKWKFKG
RDIYTFDGALNKSTVPTDESSAKIEVSQLLKGDASLKMDKSDAVSHTGNYTCEVTELTREGE
TIIELKYRVVSWESPNENILIVIFPIFAILLEWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKEVASNQKTIQPPRKAVEEPL
N

Exemplary CD47 polypeptides excluding the signal peptide are provided by SEQ ID NOs: 5-8. Suitably, a CD47 polypeptide comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to any of SEQ ID NOs: 5-8. Suitably, a CD47 polypeptide comprises or consists of the amino acid sequence of any of SEQ ID NOs: 5-8.

Exemplary CD47 polypeptide excluding signal peptide
(SEQ ID NO: 5)
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVVSWESPNEN
ILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPM
HGPLLISGLSILALAQLLGLVYMKFVASNQKTIQPPRNN
Exemplary CD47 polypeptide excluding signal peptide
(SEQ ID NO: 6)
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVVSWESPNEN
ILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPM
HGPLLISGLSILALAQLLGLVYMKEVASNQKTIQPPRKAVEEPLNAFKESKGMMNDE
Exemplary CD47 polypeptide excluding signal peptide
(SEQ ID NO: 7)
QLLFNKTKSVEFTFCNDTVVIPCFVTNMEAQNTTEVYVKWKFKGRDIYTEDGALNKSTVPTD
FSSAKIEVSQLLKGDASLKMDKSDAVSHTGNYTCEVTELTREGETIIELKYRVVSWESPNEN
ILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPM
HGPLLISGLSILALAQLLGLVYMKFV
Exemplary CD47 polypeptide excluding signal peptide
(SEQ ID NO: 8)
QLLFNKTKSVEFTFCNDTVVIPCFVTNMEAQNTTEVYVKWKFKGRDIYTEDGALNKSTVPTD
FSSAKIEVSQLLKGDASLKMDKSDAVSHTGNYTCEVTELTREGETIIELKYRVVSWESPNEN
ILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPM
HGPLLISGLSILALAQLLGLVYMKFVASNQKTIQPPRKAVEEPLN

A person skilled in the art would be able to generate variants and/or fragments based on conservative substitutions and/or the known structural and functional features of CD47. These are described, for instance in Fenalti, G., et al., 2021. Nature communications, 12(1), pp. 1-14.

Suitably, a fragment of CD47 and/or CD47 variant retains the ability to inhibit phagocytosis. Suitably, a CD47 fragment and/or CD47 variant may comprise the extracellular domain of CD47. The extracellular domain of human CD47 may interact with SIRP-α and inhibit phagocytosis. Optionally, a CD47 fragment and/or CD47 variant comprises the transmembrane domain of CD47. The domains may be linked by inter-domain linker(s). The fragment and/or variant may have at least 40%, at least 50%, at least 60%, at least 70%, at least 80%, at least 90%, at least 95%, or at least 100% of the activity of a full-length CD47 polypeptide.

Suitably, a variant of SEQ ID NO: 1 may comprise one or more variation selected from V5I, C14W, C15R, F22L, S27F, F30L, F32Y, T36S, V38L, V38I, F42V, T44A, N50S, T51A, T52S, T52A, V56I, R63K, A71T, S75Y, T76A, P78L, P78S, P78A, S82R, S82N, S83T, K85N, K85E, V88A, V88L, V88I, Q90R, L91F, K93N, M100I, M100V, D101G, K102R, K102T, S107L, I126F, I127V, K130Q, R132H, S138F, V146, I150V, I153V, S169A, G170R, G170S, G171S, D173Y, I177V, A178G, V181I, V185A, I186V, V188A, I189T, I191V, V198I, A207S, T215I, I219M, Y226C, A231S, T235A, S236F, A240V, A240T, I241V, V243I, I244T, V246L, Y249F, A252S, A252T, V254A, S257T, I264M, I264L, M266I, M266T, M266V, V287I, V292A, N295S, N295D, Q296L, P302S, N304S, and N304D. These are considered to be tolerated, benign, and/or likely benign variations as predicted by SIFT, PolyPhen, CADD, REVEL, and MetaLR.

Suitably, a variant of SEQ ID NO: 2 may comprise one or more variation selected from P3L, A6P, F22L, S27F, F30L, F32Y, T36S, V38L, V38I, F42V, N50S, T51A, T52S, T52A, V56I, R63K, A71T, S75Y, T76A, P78L, P78S, P78A, S82R, S82N, S83T, K85N, K85E, V88A, V88L, V88I, Q90R, L91F, K93N, M100I, M100V, D101G, K102R, K102T, S107L, I126F, I127V, K130Q, R132H, S138F, V146I, I150V, I153V, S169A, G170R, G170S, G171S, I177V, A178G, V181I, I186V, V188A, I189T, I191V, V198I, A207S, T215I, I219M, Y226C, A231S, T235A, A240V, A240T, I241V, V243I, I244T, V246L, Y249F, A252S, A252T, V254A, I264M, I264L, M266I, M266T, M266V, V287I, V292A, N295S, N295D, and Q296L. These are considered to be tolerated, benign, and/or likely benign variations as predicted by SIFT, PolyPhen, CADD, REVEL, and MetaLR.

An exemplary CD47 fragment is provided by SEQ ID NO: 9. Suitably, a CD47 fragment comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to SEQ ID NO: 9. Suitably, a CD47 fragment comprises or consists of the amino acid sequence of SEQ ID NO: 9.

Exemplary CD47 fragment
(SEQ ID NO: 9)
MWPLVAALLLGSACCGSAQLLENKTKSVEFTFCNDTVVIPCFVTNMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPN

An exemplary CD47 fragment excluding the signal peptide is provided by SEQ ID NO: 10. Suitably, a CD47 fragment comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to SEQ ID NO: 10. Suitably, a CD47 fragment comprises or consists of the amino acid sequence of SEQ ID NO: 10.

Exemplary CD47 fragment excluding signal peptide
(SEQ ID NO: 10)
QLLENKTKSVEFTFCNDTVVIPCFVTNMEAQNTTEVYVKWKFKGRDIYTEDGALNKSTVPTD
FSSAKIEVSQLLKGDASLKMDKSDAVSHTGNYTCEVTELTREGETIIELKYRVVSWFSPN

MHC-I^{low} or MHC-I^{free} Lentiviral Vectors

The lentiviral vector of the present invention may be a MHC-I^{low} lentiviral vector or a MHC-I^{free} lentiviral vector. In preferred embodiments, the lentiviral vector of the present invention is a MHC-I^{free} lentiviral vector.

As used herein, a “MHC-I^{low} lentiviral vector” may refer to a lentiviral vector with reduced levels of one or more MHC-I molecules on its surface (i.e. reduced levels of surface-exposed MHC-I molecules). The number of surface-exposed MHC-I molecules may be reduced such that the immune response to the MHC-I is decreased to a therapeutically relevant degree.

As used herein, a “MHC-I^{free} lentiviral vector” may refer to a lentiviral vector which is substantially devoid of (or free of) one or more MHC-I molecules on its surface (i.e. substantially devoid of (or free of) surface-exposed MHC-I molecules).

The major histocompatibility complex class I (MHC-I) is a heterodimeric membrane protein that is displayed on the outer leaflet of the cell membrane (see e.g. Penn, D. J. and Ilmonen, P., 2005. Major histocompatibility complex (MHC). eLS). MHC-I functions to bind and display peptide fragments of proteins to the extracellular environment where they may be recognised by CD8+ cytotoxic T cells. Peptide fragments generated from normal cellular proteins will not activate cytotoxic T cells due to central and peripheral tolerance mechanisms. However, foreign peptides (e.g. those originating from viral proteins) will cause activation of an immune response to destroy the cell. An allogeneic MHC-I protein itself may be recognised by the immune system. For example, antibodies may bind MHC-I epitopes directly. As a result, lentiviral vectors that comprise MHC-I molecules originating from an allogeneic source may be targeted and neutralised by the immune system.

The term “MHC-I molecules” may refer to human MHC-I molecules. Human MHC-I is also referred to as human leukocyte antigen class I (HLA-1) and is expressed on almost all nucleated cells. HLA-I consists of two polypeptide chains, an HLA-I heavy chain (a chain) and P2 microglobulin (P2M or p chain). The HLA-I α chain and P2M are linked non-covalently. The HLA-I α chain is polymorphic. Six HLA-I α chains have been identified to date, including three classical, highly polymorphic α chains (HLA-A, HLA-B and HLA-C) and three non-classical, less polymorphic (HLA-E, HLA-F and HLA-G) α chains. The MHC-I molecules may comprise or consist of HLA-A, HLA-B, and HLA-C molecules, which comprise an invariant P2M sequence.

The term “MHC-I molecules” may also include variant MHC-I sequences, such as polymorphisms of HLA-I α chain sequences and/or P2M sequences. For example, variant MHC-I sequences may include HLA-I α chain sequences and/or P2M sequences with single nucleotide polymorphisms (SNPs) or multiple SNPs.

Any suitable assay to quantify the amount of MHC-I molecules present on the surface of the lentiviral vector may be used.

In some embodiments, the amount of MHC-I molecules may be determined by immunostaining for MHC-I and electron microscopy, as described in Milani, M., et al., 2017. EMBO molecular medicine, 9(11), pp. 1558-1573. The MHC-I molecules may be detected in an amount of less than about 10 gold particles/lentiviral particle, less than about 9 gold particles/lentiviral particle, less than about 8 gold particles/lentiviral particle, less than about 7 gold particles/lentiviral particle, less than about 6 gold particles/lentiviral particle, less than about 5 gold particles/lentiviral particle, less than about 4 gold particles/lentiviral particle, less than about 3 gold particles/lentiviral particle, less than about 2 gold particles/lentiviral particle, less than about 1 gold particle/lentiviral particle, or about 0 gold particles/lentiviral particle. The MHC-I molecules may be undetectable (e.g. the amount of gold particles detected may not be significantly higher than background levels).

The lentiviral vector of the present invention may be obtained from a MHC-I^{low} host cell or a MHC-I^{free} host cell. In preferred embodiments, the lentiviral vector of the present invention is obtained from a MHC-I^{free} host cell. As used herein, a “MHC-I^{low} host cell” may refer to a host cell with reduced levels of one or more MHC-I molecule on its surface. As used herein, a “MHC-I^{free} host cell” may refer to a host cell which is substantially devoid of or free of one or more MHC-I molecule on its surface.

A MHC-I^{low} or MHC-I^{free} host cell may be genetically engineered to decrease expression of MHC-I on the cell surface. For example, the cell may comprise a genetically engineered disruption of a gene encoding P2-microglobulin and/or a genetically engineered disruption of a gene encoding an MHC-I α chain.

Methods for genetic engineering to decrease protein expression are known in the art. For example, this may be achieved by targeted gene knockout. To decrease protein expression, the gene encoding the protein itself or its regulatory sequence (e.g. its promoter) may be knocked out. Knockout may be achieved by deletion of a section of the coding nucleic acid sequence, which may delete a section of the protein essential for expression or stability, or alter the reading frame of the coding sequence or by base-editing. Suitable methods for targeted gene knockout include use of zinc finger nucleases (ZFNs), transcription activator-like effector nucleases (TALENs) and CRISPR/Cas-based RNA-guided nucleases (see e.g. Gaj, T. et al., 2013. Trends Biotechnol, 31, pp. 397-405). For example, the CRISPR/Cas9 RNA-guided nuclease may be used to catalyse a double strand break at a specific locus in the genome if provided with appropriate RNA guides designed to bind that locus. Cas9 and the guide RNA may be delivered to a target cell by transfection of vectors encoding the protein and RNA. Cells attempt to repair any double strand breaks in their DNA using the non-homologous end joining (NHEJ) pathway. This is an error-prone mechanism which inserts random nucleotides and often disrupts the reading frame of the targeted gene. Alternatively, the genetic engineering to decrease protein expression may be accomplished using RNAi techniques, microRNA or antisense RNA to suppress expression of the target gene.

Once the targeted gene knockout or suppression of expression approach has been carried out, the resulting population of cells may be screened to select and enrich for those cells exhibiting the phenotype of interest, for example decreased expression of surface-exposed MHC-I. Suitable techniques for screening and enrichment are known in the art and include flow cytometry and fluorescence-activated cell sorting (FACS).

In some embodiments, the host cell comprises a genetically engineered disruption of a gene encoding β2-microglobulin. β2-microglobulin stabilises MHC-1, thus cells deficient in P2-microglobulin will exhibit decreased expression of MHC-I on the surface of the cell. The cell may comprise genetically engineered disruptions in all copies of the gene encoding P2-microglobulin.

In another embodiment, the cell comprises a genetically engineered disruption of one or more gene encoding an MHC-I α chain. The cell may comprise genetically engineered disruptions in all copies of the gene encoding an MHC-I α chain.

The cell may comprise both genetically engineered disruptions of genes encoding P2-microglobulin and genetically engineered disruptions of genes encoding an MHC-I α chain.

Decreased expression of MHC-I on the surface of the cell may refer to a decrease in the number of MHC-I molecules that are expressed on the surface of the cell that has been genetically engineered, in comparison to the number of MHC-I molecules that are expressed on the surface of a cell lacking the genetic engineering, but under otherwise substantially identical conditions. The expression of MHC-I on the surface of the cell may be decreased such that the number of surface-exposed MHC-I molecules is, for example, less than about 50%, 40%, 30%, 25%, 20%, 15%, 10%, 5%, 4%, 3%, 2% or 1% of the number of surface-exposed MHC-I molecules that are displayed in the absence of the genetic engineering. In some embodiments, the expression of MHC-I on the surface of the cell is decreased such that the number of surface-exposed MHC-I molecules is 0% of the number of surface-exposed MHC-I molecules that are displayed in the absence of the genetic engineering.

The expression of MHC-I on the surface of the cell is preferably decreased such that the cell is substantially devoid of surface-exposed MHC-I molecules. In this context, “substantially devoid” may mean that there is a substantial decrease in the number of MHC-I molecules that are expressed on the surface of the cell that has been genetically engineered, in comparison to the number of MHC-I molecules that are expressed on the surface of a cell lacking the genetic engineering, such that the immune response to MHC-I on lentiviral vectors produced by the cell is decreased to a therapeutically useful degree.

Suitably, the lentiviral vector of the present invention has a lower concentration of MHC-I molecules on its surface than a lentiviral vector obtained from an unmodified host cell (e.g. an unmodified producer cell or packaging cell, as described herein). Suitably, the lentiviral vector has less than about 50%, 40%, 30%, 25%, 20%, 15%, 10%, 5%, 4%, 3%, 2% or 1% of the number of surface-exposed MHC-I molecules that are displayed on a lentiviral vector obtained from an unmodified host cell. In some embodiments, the lentiviral vector has less than about 20% of the number of surface-exposed MHC-I molecules that are displayed on a lentiviral vector obtained from an unmodified host cell.

In some embodiments, the lentiviral vector of the present invention is substantially devoid of MHC-I molecules on its surface. In this context, “substantially devoid” may mean that there is no detectable immune response due to the molecules on the surface of the lentiviral vector.

In some embodiments, the lentiviral vector of the present invention is free of MHC-I molecules on its surface. In this context, “free” may mean that there are no detectable molecules (e.g. by immunostaining and electron microscopy) on the surface of the lentiviral vector. As used herein, “not detectable” may refer to levels which are not statistically significantly different compared to background levels.

In some embodiments, the lentiviral vector of the present invention has decreased HLA-A, HLA-B, and/or HLA-C molecules on its surface. Suitably, the lentiviral vector has less than about 50%, 40%, 30%, 25%, 20%, 15%, 10%, 5%, 4%, 3%, 2% or 1% of the number of surface-exposed HLA-A molecules that are displayed on a lentiviral vector obtained from an unmodified host cell. Suitably, the lentiviral vector has less than about 50%, 40%, 30%, 25%, 20%, 15%, 10%, 5%, 4%, 3%, 2% or 1% of the number of surface-exposed HLA-B molecules that are displayed on a lentiviral vector obtained from an unmodified host cell. Suitably, the lentiviral vector has less than about 50%, 40%, 30%, 25%, 20%, 15%, 10%, 5%, 4%, 3%, 2% or 1% of the number of surface-exposed HLA-C molecules that are displayed on a lentiviral vector obtained from an unmodified host cell.

In some embodiments, the lentiviral vector of the present invention is substantially devoid of HLA-A, HLA-B, and/or HLA-C molecules on its surface. In some embodiments, the lentiviral vector of the present invention is substantially devoid of HLA-A, HLA-B, and HLA-C molecules on its surface. In some embodiments, the lentiviral vector of the present invention is free of HLA-A, HLA-B, and/or HLA-C molecules on its surface. In some embodiments, the lentiviral vector of the present invention is free of HLA-A, HLA-B, and HLA-C molecules on its surface.

As described above, an HLA-1 molecule consists of two polypeptide chains, an HLA-1 heavy chain (α chain) and P2 microglobulin (P2M or p chain). The HLA-1 α chain and P2M are linked non-covalently.

The skilled person would readily be able to determine amino acid and nucleic acid sequences of HLA-1 α chains. For example, the HLA-1 α chains may be identified in a genome sequence using their location within the major histocompatibility complex region of the chromosome (see e.g. Penn, D. J. and Ilmonen, P., 2005. Major histocompatibility complex (MHC). eLS).

HLA-A alpha chains may have an amino acid sequence of UniProtKB P04439. Exemplary HLA-A alpha chains are provided by SEQ ID NOs: 11 and 12. Suitably, an HLA-A alpha chain comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to SEQ ID NO: 11 or 12. Suitably, a HLA-A alpha chain comprises or consists of the amino acid sequence of SEQ ID NO: 11 or 12.

Exemplary HLA-A alpha chain
(SEQ ID NO: 11)
MAVMAPRTLLLLLSGALALTQTWAGSHSMRYFFTSVSRPGRGEPRFIAVGYVDDTQFVRFDS
DAASQRMEPRAPWIEQEGPEYWDQETRNVKAQSQTDRVDLGTLRGYYNQSEAGSHTIQIMYG
CDVGSDGRFLRGYRQDAYDGKDYIALNEDLRSWTAADMAAQITKRKWEAAHEAEQLRAYLDG
TCVEWLRRYLENGKETLQRTDPPKTHMTHHPISDHEATLRCWALGFYPAEITLTWQRDGEDQ
TQDTELVETRPAGDGTFQKWAAVVVPSGEEQRYTCHVQHEGLPKPLTLRWELSSQPTIPIVG
IIAGLVLLGAVITGAVVAAVMWRRKSSDRKGGSYTQAASSDSAQGSDVSLTACKV
Exemplary HLA-A alpha chain
(SEQ ID NO: 12)
MAVMAPRTLLLLLSGALALTQTWAGSHSMRYFFTSVSRPGRGEPRFIAVGYVDDTQFVRFDS
DAASQRMEPRAPWIEQEGPEYWDQETRNVKAQSQTDRVDLGTLRGYYNQSEAGSHTIQIMYG
CDVGSDGRFLRGYRQDAYDGKDYIALNEDLRSWTAADMAAQITKRKWEAAHAAEQQRAYLEG
RCVEWLRRYLENGKETLQRTDPPKTHMTHHPISDHEATLRCWALGFYPAEITLTWQRDGEDQ
TQDTELVETRPAGDGTFQKWAAVVVPSGEEQRYTCHVQHEGLPKPLTLRWELSSQPTIPIVG
IIAGLVLLGAVITGAVVAAVMWRRKSSGGEGVKDRKGGSYTQAASSDSAQGSDVSLTACKV

HLA-B alpha chains may have an amino acid sequence of UniProtKB P01889. An exemplary HLA-B alpha chain is provided by SEQ ID NO: 13. Suitably, an HLA-B alpha chain comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to SEQ ID NO: 13. Suitably, a HLA-B alpha chain comprises or consists of the amino acid sequence of SEQ ID NO: 13.

Exemplary HLA-B alpha chain
(SEQ ID NO: 13)
MLVMAPRTVLLLLSAALALTETWAGSHSMRYFYTSVSRPGRGEPRFISVGYVDDTQFVRFDS
DAASPREEPRAPWIEQEGPEYWDRNTQIYKAQAQTDRESLRNLRGYYNQSEAGSHTLQSMYG
CDVGPDGRLLRGHDQYAYDGKDYIALNEDLRSWTAADTAAQITQRKWEAAREAEQRRAYLEG
ECVEWLRRYLENGKDKLERADPPKTHVTHHPISDHEATLRCWALGFYPAEITLTWQRDGEDQ
TQDTELVETRPAGDRTFQKWAAVVVPSGEEQRYTCHVQHEGLPKPLTLRWEPSSQSTVPIVG
IVAGLAVLAVVVIGAVVAAVMCRRKSSGGKGGSYSQAACSDSAQGSDVSLTA

HLA-C alpha chains may have an amino acid sequence of UniProtKB P10321. Exemplary HLA-C alpha chains are provided by SEQ ID NOs: 14 and 15. Suitably, an HLA-C alpha chain comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to SEQ ID NO: 14 or 15. Suitably, a HLA-C alpha chain comprises or consists of the amino acid sequence of SEQ ID NO: 14 or 15.

Exemplary HLA-C alpha chain
(SEQ ID NO: 14)
MRVMAPRALLLLLSGGLALTETWACSHSMRYEDTAVSRPGRGEPRFISVGYVDDTQFVRFDS
DAASPRGEPRAPWVEQEGPEYWDRETQKYKRQAQADRVSLRNLRGYYNQSEDGSHTLQRMSG
CDLGPDGRLLRGYDQSAYDGKDYIALNEDLRSWTAADTAAQITQRKLEAARAAEQLRAYLEG
TCVEWLRRYLENGKETLQRAEPPKTHVTHHPLSDHEATLRCWALGFYPAEITLTWQRDGEDQ
TQDTELVETRPAGDGTFQKWAAVVVPSGQEQRYTCHMQHEGLQEPLTLSWEPSSQPTIPIMG
IVAGLAVLVVLAVLGAVVTAMMCRRKSSGGKGGSCSQAACSNSAQGSDESLITCKA
Exemplary HLA-C alpha chain
(SEQ ID NO: 15)
MRVMAPRALLLLLSGGLALTETWACSHSMRYFDTAVSRPGRGEPRFISVGYVDDTQFVRFDS
DAASPRGEPRAPWVEQEGPEYWDRETQKYKRQAQADRVSLRNLRGYYNQSEDGSHTLQRMSG
CDLGPDGRLLRGYDQSAYDGKDYIALNEHLRSCTAADTAAQITQRKLEAARAAEQLRAYLEG
TCVEWLRRYLENGKETLQRAEPPKTHVTHHPLSDHEATLRCWALGFYPAEITLTWQRDGEDQ
TQDTELVETRPAGDGTFQKWAAVVVPSGQEQRYTCHMQHEGLQEPLTLRWGGKGGSCSQAAC
SNSAQGSDESLITCKA

Amino acid and nucleic acid sequences encoding P2M are also known in the art. For example, a nucleic acid sequence of a human P2M is deposited as GenBank Accession No. NM_004048.

An HLA p chain may be that of UniProtKB P61769. An exemplary HLA p chain is provided by SEQ ID NO: 16. Suitably, a HLA p chain comprises an amino acid sequence having at least about 70%, at least about 75%, at least about 80%, at least about 85%, at least about 90%, at least about 95%, at least about 96%, at least about 97%, at least about 98%, at least about 99%, or about 100% identity to SEQ ID NO: 16. Suitably, a HLA p chain comprises or consists of the amino acid sequence of SEQ ID NO: 16.

Exemplary HLA beta chain
(SEQ ID NO: 16)
MSRSVALAVLALLSLSGLEAIQRTPKIQVYSRHPAENGKSNFLNCYVSGFHPSDIEVDLLKN
GERIEKVEHSDLSFSKDWSFYLLYYTEFTPTEKDEYACRVNHVTLSQPKIVKWDRDM

The lentiviral vector of the present invention may be a CD47^{high}/MHC-I^{free} lentiviral vector or a CD47^{high}/MHC-I^{low} lentiviral vector. In preferred embodiments, the lentiviral vector of the present invention is a CD47^{high}/MHC-I^{free} lentiviral vector.

The lentiviral vector of the present invention may be obtained from a CD47^{high}/MHC-I^{free} host cell or a CD47^{high}/MHC-I^{low} host cell. In preferred embodiments, the lentiviral vector of the present invention is obtained from a CD47^{high}/MHC-I^{free} host cell.

miRNA Target Sequence

The lentiviral vector of the present invention may comprise one or more miRNA target sequences. The one or more miRNA target sequences may be operably linked to the protein-coding sequence. The term “operably linked” may mean that the components described are in a relationship permitting them to function in their intended manner.

MicroRNA (miRNA) genes are scattered across all human chromosomes, except for the Y chromosome. Similar to protein-coding genes, miRNAs are usually transcribed from polymerase-II promoters, generating a so-called primary miRNA transcript (pri-miRNA). From the pri-miRNA, a stem loop of about 60 nucleotides in length, called miRNA precursor (pre-miRNA), is excised leaving a 5′ phosphate and a 2 bp long, 3′ overhang. The pre-miRNA is then actively transported from the nucleus to the cytoplasm. Then, Dicer performs a double strand cut at the other end of the stem loop, generating a 19-24 bp duplex, which is composed of the mature miRNA and the opposite strand of the duplex, called miRNA*. One strand of the duplex is selectively loaded into the RNA-induced silencing complex (RISC), and accumulates as the mature microRNA. This strand is usually the one whose 5′ end is less tightly paired to its complement. However, there are some miRNAs that support accumulation of both duplex strands to similar extent.

Once loaded into RISC, the guide strand of the mature microRNA interacts with mRNA target sequences preferentially found in the 3′ untranslated region (3′UTR) of protein-coding genes. If the whole guide strand sequence is perfectly complementary to the mRNA target, the mRNA is endonucleolytically cleaved. If only the seed sequence (i.e. nucleotides 2-8 counted from the 5′ end of the miRNA) is perfectly complementary to the target mRNA, RNAi may act through alternative mechanisms leading to translational repression.

Expression of the protein from the protein-coding sequence (i.e. “transgene expression”) may be regulated by one or more endogenous miRNAs using one or more corresponding miRNA target sequences. Using this method, one or more miRNAs endogenously expressed in a cell prevent or reduce transgene expression in that cell by interacting with its corresponding miRNA target sequence positioned in the lentiviral genome (see e.g. Brown, B. D. et al. (2007) Nat Biotechnol 25: 1457-1467).

Suitable miRNA target sequences which suppress transgene expression in specific cells will be known to the skilled person. Determining a miRNA with the desired expression profile may be achieved using techniques known to those skilled in the art. For example, a mammalian microRNA expression atlas is described in Landgraf, P., et al., 2007. Cell, 129(7), pp. 1401-1414 and the distribution of miRNA expression across human tissues is described in Ludwig, N., et al., 2016. Nucleic acids research, 44(8), pp. 3865-3877. Once a miRNA has been identified, the corresponding target sequence can readily be determined using, for example, a microRNA database, such as miRBase (Griffiths-Jones, S., et al., 2007. Nucleic acids research, 36(suppl_1), pp.D154-D158).

A miRNA target sequence may be fully or partially complementary to the corresponding miRNA. The term “fully complementary”, as used herein, may mean that the target sequence has a nucleic acid sequence which is 100% complementary to the sequence of the miRNA which recognises it. The term “partially complementary”, as used herein, may mean that the target sequence is only in part complementary to the sequence of the miRNA which recognises it, whereby the partially complementary sequence is still recognised by the miRNA. In other words, a partially complementary target sequence in the context of the present invention is effective in recognising the corresponding miRNA and effecting prevention or reduction of transgene expression in cells expressing that miRNA. Suitably, a partially complementary miRNA target sequence may be fully complementary to the miRNA seed sequence.

Including more than one copy of a miRNA target sequence in a lentiviral vector may increase the effectiveness of the system. Also, different miRNA target sequences can be included. For example, the protein-coding sequence may be operably linked to more than one miRNA target sequence, which may or may not be different. The miRNA target sequences may be in tandem, but other arrangements are envisaged. The lentiviral vector may, for example, comprise 1, 2, 3, 4, 5, 6, 7 or 8 copies of the same or different miRNA target sequences. Suitably, the lentiviral vector comprises 4 miRNA target sequences of each miRNA target sequence.

Copies of miRNA target sequences may be separated by a spacer sequence. A spacer sequence may comprise, for example, at least one, at least two, at least three, at least four or at least five nucleotide bases.

Suitably, the lentiviral vector comprises one or more miRNA target sequence, two or more miRNA target sequences, three or more miRNA target sequences, or four or more miRNA target sequences. Suitably, the protein-coding sequence is operably linked to one or more miRNA target sequence, two or more miRNA target sequences, three or more miRNA target sequences, or four or more miRNA target sequences. In some embodiments, the protein-coding sequence is operably linked to four miRNA target sequences.

The miRNA target sequence may be a human miRNA target sequence. Suitably, the miRNA target sequence is a −5p or −3p miRNA target sequence.

The one or more miRNA target sequence may suppress transgene expression in one or more cells other than liver cells (e.g. hepatocytes).

The one or more miRNA target sequence may suppress transgene expression in hematopoietic-lineage cells. Hematopoietic stem cells give rise to different types of blood cells, in lines called myeloid and lymphoid. As used herein, “hematopoietic-lineage cells” may include myeloid cells and lymphoid cells. Myeloid cells may include monocytes, macrophages, neutrophils, basophils, and eosinophils. Lymphoid cells may include T cells, B cells, natural killer cells, and innate lymphoid cells.

The one or more miRNA target sequences may suppress transgene expression in antigen-presenting cells. As used herein, an “antigen presenting cell” (APC) may refer to a cell that displays antigen bound by major histocompatibility complex (MHC) proteins on its surface. APCs may be hematopoietic-lineage cells. The antigen-presenting cells may be professional antigen-presenting cells. Professional APCs specialise in presenting antigens to T cells and may include macrophages, B cells and dendritic cells. Suitably, the APCs are splenic and/or hepatic APCs.

The one or more miRNA target sequences may suppress transgene expression in hematopoietic-lineage antigen-presenting cells.

By preventing transgene expression in antigen-presenting cells, while permitting high levels of expression in other cells, miRNA regulation may enable strong and stable gene transfer in the absence of an immune response.

As used herein, the term “suppress expression” may refer to a reduction of expression in the relevant cell type(s) of a transgene to which the one or more miRNA target sequence is operably linked as compared to transgene expression in the absence of the one or more miRNA target sequence, but under otherwise substantially identical conditions. In some embodiments, transgene expression is suppressed by at least 50%. In some embodiments, transgene expression is suppressed by at least 60%, at least 70%, at least 80%, at least 90%, or at least 95%. In some embodiments, transgene expression is substantially prevented, e.g. not detectable.

The miRNA-mediated approach for restricting gene expression has several advantages over other strategies of regulating transgenes. Although using tissue-specific promoters can successfully limit expression to target cells, leaky expression in a fraction of non-target cells is observed. This occurs because the reconstituted promoter, modified for inclusion into a vector system, often loses some of its cell specificity and also because vector integration near active promoters and enhancers can activate the tissue-specific promoter and drive transgene expression. In contrast, because miRNA-mediated silencing occurs at the post-transcriptional level, promoter and enhancer trapping is irrelevant. As such, miRNA-regulation can be used to effectively de-target transgene expression from a particular cell type, while still allowing for broad tissue expression. miRNA regulation may also be used as in combination with tissue-specific promoter/enhancers. By including the miRNA target sequence in expression cassettes already under the control of a tissue-specific promoter, an additional layer of regulation is added which may eliminate off-target expression.

Exemplary miRNA target sequences which suppress transgene expression in hematopoietic-lineage cells and/or antigen-presenting cells, include, but are not limited to, miR-181, miR-142, miR-223, and miR-155 target sequences. Other miRNA target sequences which suppress transgene expression in hematopoietic-lineage cells and/or antigen-presenting cells are known in the art (see e.g. Ghafouri-Fard, S., et al., 2021. Non-coding RNA research, 6(1), pp. 8-14). miRNAs which are expressed in hematopoietic-lineage cells and/or antigen-presenting cells interact with the corresponding miRNA target sequence and reduce the expression of the target gene (see e.g. Brown, B. D., et al., 2006. Nature medicine, 12(5), pp. 585-591 and Brown, B. D., et al., 2007. Nature biotechnology, 25(12), pp. 1457-1467).

Further miRNA target sequences that suppress transgene expression in hematopoietic-lineage cells and/or antigen-presenting cells can be identified by any suitable method, for example miRNA expression analysis as described in Monticelli, S., et al., 2005. Genome biology, 6(8), pp. 1-15.

Suitably, the one or more miRNA target sequence comprise or consist of: (i) one or more (e.g. 1, 2, 3, 4, 5, 6, 7, 8) miR-142 target sequence; (ii) one or more (e.g. 1, 2, 3, 4, 5, 6, 7, 8) miR-181 target sequence; (iii) one or more (e.g. 1, 2, 3, 4, 5, 6, 7, 8) miR-223 target sequence; and/or (iv) one or more (e.g. 1, 2, 3, 4, 5, 6, 7, 8) miR-155 target sequence.

In some embodiments, the one or more miRNA target sequence comprise or consist of: (i) two or more miR-142 target sequences; (ii) two or more miR-181 target sequences; (iii) two or more miR-223 target sequences; and/or (iv) two or more miR-155 target sequences. In some embodiments, the one or more miRNA target sequence comprise or consist of: (i) at least four miR-142 target sequences; (ii) at least four miR-181 target sequences; (iii) at least four miR-223 target sequences; and/or (iv) at least four miR-155 target sequences. In some embodiments, the one or more miRNA target sequence comprise or consist of: (i) four miR-142 target sequences; (ii) four miR-181 target sequences; (iii) four miR-223 target sequences; and/or (iv) four miR-155 target sequences. Suitably, the target sequences are separated by 10 spacer sequences.

In some embodiments, the one or more miRNA target sequence comprise or consist of one or more (e.g. 1, 2, 3, 4, 5, 6, 7, 8) miR-142 target sequence. In some embodiments, the one or more miRNA target sequence comprise or consist of two or more miR-142 target sequences. In some embodiments, the one or more miRNA target sequence comprise or consist of three or more miR-142 target sequences. In some embodiments, the one or more miRNA target sequence comprise or consist of four or more miR-142 target sequences. In some embodiments, the one or more miRNA target sequence comprise or consist of four miR-142 target sequences. Suitably, the target sequences are separated by spacer sequences.

The miR-142 target sequence may be a human miRNA target sequence. Suitably, the miR-142 target sequence is a miR-142-5p or miR-142-3p miRNA target sequence. In some embodiments, the miR-142 target sequence is a miR-142-3p miRNA target sequence.

In some embodiments, the miR-142 target sequence comprises or consists of a nucleotide sequence which is at least 80% identical to SEQ ID NO: 17 or a fragment thereof. Suitably, the miR-142 target sequence comprises or consists of a nucleotide sequence which is at least 85%, at least 90%, or at least 95% identical to SEQ ID NO: 17 or a fragment thereof.

In some embodiments, the miR-142 target sequence comprises or consists of the nucleotide sequence SEQ ID NO: 17 or a fragment thereof.

Exemplary miR-142 target sequence
(SEQ ID NO: 17)
TCCATAAAGTAGGAAACACTACA

In some embodiments, the one or more miRNA target sequence comprises or consists of a nucleotide sequence which is at least 70% identical to SEQ ID NO: 18 or a fragment thereof. Suitably, the one or more miRNA target sequence comprises or consists of a nucleotide sequence which is at least 75%, at least 75%, at least 80%, at least 85%, at least 90%, at least 95%, at least 96%, at least 97%, at least 98%, at least 99%, or 100% identical to SEQ ID NO: 18 or a fragment thereof.

In some embodiments, the one or more miRNA target sequence comprises or consists of the nucleotide sequence of SEQ ID NO: 18 or a fragment thereof.

(SEQ ID NO: 18)
TCCATAAAGTAGGAAACACTACACGATTCCATAAAGTAGGAAACA
CTACAACCGGTTCCATAAAGTAGGAAACACTACATCACTCCATAA
AGTAGGAAACACTACA
Exemplary 4x miR-142 target sequence

### MMA-Associated Polypeptide

The protein-coding sequence delivered by the lentiviral vector of the present invention is a nucleotide sequence encoding a methylmalonic acidemia (MMA)-associated polypeptide. Once delivered to a cell, the protein encoded by the protein-coding sequence may be expressed in the cell (i.e. “transgene expression” may occur).

As used herein, a “MMA-associated polypeptide” may be any polypeptide associated with methylmalonic acidemia (MMA). As described above, MMA is a group of inborn errors of metabolism associated with elevated methylmalonic acid concentration in the blood and urine that result from the failure to convert methylmalonyl-CoA into succinyl-CoA, during propionyl-CoA metabolism in the mitochondrial matrix (see e.g. Manoli I, et al. 2005. Isolated Methylmalonic Acidemia. In GeneReviews). A MMA-associated polypeptide may be any polypeptide associated with the pathway of the conversion of methylmalonyl-CoA into succinyl-CoA, including polypeptides associated with intracellular cobalamin metabolism (e.g. a polypeptide encoded by MMUT, MMAA, MMAB, MMACHC, MMADHC, LMBRD1, ABCD4, HCFC1, MCEE, SUCLA2/SUCLG1, ACSF3, or ALDH6A1).

In preferred embodiments, the MMA-associated polypeptide is an iMMA-associated polypeptide. As described above, isolated MMA (iMMA) may be caused by a complete (mut⁰ enzymatic subtype) or partial (mut⁻ enzymatic subtype) deficiency of the enzyme methylmalonyl-CoA mutase (encoded by MMUT); a defect in the transport or synthesis of its cofactor, adenosyl-cobalamin (caused by pathogenic variants in MMAA, MMAB, or MMADHC); or a deficiency of the enzyme methylmalonyl-CoA epimerase (encoded by MCEE) (see e.g. Manoli I, et al. 2005. Isolated Methylmalonic Acidemia. In GeneReviews).

Suitably, the MMA-associated polypeptide is selected from methylmalonyl-CoA mutase (MMUT), or a fragment and/or variant thereof; methylmalonic aciduria type A (MMAA), or a fragment and/or variant thereof; methylmalonic aciduria type B (MMAB), or a fragment and/or variant thereof; methylmalonic aciduria and homocystinuria type D (MMADHC), or a fragment and/or variant thereof; and methylmalonyl-CoA epimerase (MCEE), or a fragment and/or variant thereof.

The protein-coding sequence may be codon-optimised. For example, the protein-coding sequence may be codon-optimised for expression in a mammalian (e.g. human) cell.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells (e.g. humans), as well as for a variety of other organisms.

### MMUT

In some embodiments, the MMA-associated polypeptide is MMUT or a fragment and/or variant thereof.

“MMUT” or “MUT” is the abbreviated name of the polypeptide encoded by MMUT and is also known as mitochondrial methylmalonyl-CoA mutase (MCM) and methylmalonyl-CoA isomerase. MMUT catalyses the AdoCbl-dependent rearrangement of methylmalonyl-CoA (mmCoA) to succinyl-CoA (sCoA). Each MMUT monomer features a two-domain structure comprising a large substrate-binding TIM barrel connected to a small AdoCbl-binding domain via a ˜100 aa inter-domain linker, with the active site situated at the N/C-domain interface (see e.g. Froese, D. S., et al., 2010. Journal of Biological Chemistry, 285(49), pp. 38204-38213).

A fragment and/or variant of MMUT may retain MMUT activity (see e.g. EC 5.4.99.2). For example, a fragment and/or variant of MMUT may be able to catalyse the rearrangement of mmCoA to sCoA. Suitably, a fragment and/or variant of MMUT may have the same or similar activity to MMUT, e.g. may have at least 40%, at least 50%, at least 60%, at least 70%, at least 80%, at least 90%, at least 95%, or at least 100% of the activity of MMUT. Assays to determine MMUT activity are described, for instance in Janata, J., et al., 1997. Human Molecular Genetics, 6(9), pp. 1457-1464 and Forny, P., et al., 2014. Human mutation, 35(12), pp. 1449-1458.

A “fragment of MMUT” may refer to a portion or region of a full-length MMUT that has the same or similar activity as a full-length MMUT, i.e. the fragment may be a functional fragment (e.g. the fragment may catalyse the rearrangement of mmCoA to sCoA). The fragment may have at least 40%, at least 50%, at least 60%, at least 70%, at least 80%, at least 90%, at least 95%, or at least 100% of the activity of full-length MMUT.

A “MMUT variant” may include an amino acid sequence or a nucleotide sequence which may be at least 50%, at least 55%, at least 65%, at least 70%, at least 75%, at least 80%, at least 85% or at least 90% identical, optionally at least 95% or at least 97% or at least 99% identical to a wild-type MMUT. MMUT variants may have the same or similar activity to a wild-type MMUT (e.g. MMUT variants may catalyse the rearrangement of mmCoA to sCoA). MMUT variants may have at least at least 40%, at least 50%, at least 60%, at least 70%, at least 80%, at least 90%, at least 95%, or at least 100% of the activity of a wild-type MMUT.

A person skilled in the art would be able to generate a fragment of MMUT and/or MMUT variants using conservative substitutions, based on the known structural and functional features of MMUT (see e.g. Froese, D. S., et al., 2010. Journal of Biological Chemistry, 285(49), pp. 38204-38213; Thomsu, N. H. and Leadlay, P. F., 1996. Protein Science, 5(9), pp. 1922-1927; and Acquaviva, C., et al., 2005. Human mutation, 25(2), pp. 167-176) and/or based on known variants (see e.g. NCBI Gene ID: 4594 and NCBI HomoloGene: 20097).

Suitably, a fragment of MMUT and/or a MMUT variant comprises an mmCoA-binding domain and an AdoCbl-binding domain. A fragment of MMUT may also comprise a mitochondrial targeting sequence and/or a dimerization domain. The domains/sequences may be linked by inter-domain linker(s).

The MMUT gene is conserved in chimpanzee, Rhesus monkey, dog, cow, mouse, and rat. The MMUT may be a human MMUT. Suitably, the MMUT may comprise or consist of a polypeptide sequence of UniProtKB accession P22033, or a fragment and/or variant thereof.

In some embodiments, the MMUT comprises or consists of an amino acid sequence which is at least 70% identical to SEQ ID NO: 37 or a fragment thereof. Suitably, the MMUT comprises or consists of an amino acid sequence which is at least 80%, at least 85%, at least 90%, at least 95%, at least 96%, at least 97%, at least 98% or at least 99% identical to SEQ ID NO: 37 or a fragment thereof.

In some embodiments, the MMUT comprises or consists of SEQ ID NO: 37 or a fragment thereof.

(SEQ ID NO: 37)
MLRAKNQLFLLSPHYLRQVKESSGSRLIQQRLLHQQQPLHPEWAA
LAKKQLKGKNPEDLIWHTPEGISIKPLYSKRDTMDLPEELPGVKP
FTRGPYPTMYTFRPWTIRQYAGFSTVEESNKFYKDNIKAGQQGLS
VAFDLATHRGYDSDNPRVRGDVGMAGVAIDTVEDTKILFDGIPLE
KMSVSMTMNGAVIPVLANFIVTGEEQGVPKEKLTGTIQNDILKEF
MVRNTYIFPPEPSMKIIADIFEYTAKHMPKENSISISGYHMQEAG
ADAILELAYTLADGLEYSRTGLQAGLTIDEFAPRLSFFWGIGMNF
YMEIAKMRAGRRLWAHLIEKMFQPKNSKSLLLRAHCQTSGWSLTE
QDPYNNIVRTAIEAMAAVFGGTQSLHTNSFDEALGLPTVKSARIA
RNTQIIIQEESGIPKVADPWGGSYMMECLTNDVYDAALKLINEIE
EMGGMAKAVAEGIPKLRIEECAARRQARIDSGSEVIVGVNKYQLE
KEDAVEVLAIDNTSVRNRQIEKLKKIKSSRDQALAERCLAALTEC
AASGDGNILALAVDASRARCTVGEITDALKKVFGEHKANDRMVSG
AYRQEFGESKEITSAIKRVHKFMEREGRRPRLLVAKMGQDGHDRG
AKVIATGFADLGFDVDIGPLFQTPREVAQQAVDADVHAVGISTLA
AGHKTLVPELIKELNSLGRPDILVMCGGVIPPQDYEFLFEVGVSN
VFGPGTRIPKAAVQVLDDIEKCLEKKQQSV
Exemplary MMUT

Suitably, a MMUT variant may comprise one or more variation selected from N6K, N6S, Q7R, F9L, S12L, P13L, P13S, H14L, H14R, H14N, Y15N, L16V, R17S, R17K, Q18R, V19G, K20E, E21A, S22L, S22P, L27V, 128L, P38S, A44T, A45S, K48E, K49R, K54R, N55K, D58A, 160T, 160L, K75R, K75E, R76T, R76K, R76G, T78A, M79T, M79V, M79L, L811, L85F, L85V, V88M, R154H, R154C, I200V, P209S, K210E, E211D, E211D, K251N, K251E, H252Y, I274V, S288A, Q293R, H331R, E334K, M336I, M336V, S342A, A349T, I372V, S416T, C433W, C433Y, C433R, N436S, N436D, I449V, M452L, E461Q, R475K, A499P, A499T, A504V, A504T, N512K, K519E, K520R, I521V, K522R, S524G, A528V, A528G, E531Q, R532H, E539K, A542V, S543N, A550V, K571R, K595R, I597L, T598A, T598P, I601V, I601L, H605R, K606R, M608T, R610H, R610C, I634V, I671M, I671V, K687R, S691A, E715K, S719A, N720S, V721I, K730T, V733I, Q734R, D737H, E740K, K741R, and K746T. These are considered to be tolerated, benign, and/or likely benign variations as predicted by SIFT, PolyPhen, CADD, and REVEL.

Suitably, a MMUT variant may comprise one or more variation selected from I69V, R154H, A499T, K520T, K522R, R532G, R532H, and I671V. These are considered to be benign (or likely benign) variations based on clinical data.

Suitably, the nucleotide sequence encoding MMUT, or a fragment and/or variant thereof, may comprise or consist of a nucleotide sequence of NCBI reference sequence NM_000255, or a fragment and/or variant thereof.

In some embodiments of the invention, the nucleotide sequence encoding MMUT, or a fragment and/or variant thereof, comprises or consists of a nucleotide sequence which is at least 70% identical to SEQ ID NO: 38 or a fragment thereof. Suitably, the nucleotide sequence encoding MMUT, or a fragment and/or variant thereof, comprises or consists of a nucleotide sequence which is at least 80%, at least 85%, at least 90%, at least 95%, at least 96%, at least 97%, at least 98% or at least 99% identical to SEQ ID NO: 38 or a fragment thereof.

In some embodiments of the invention, the nucleotide sequence encoding MMUT, or a fragment and/or variant thereof, comprises or consists of the nucleotide sequence SEQ ID NO: 38 or a fragment thereof.

(SEQ ID NO: 38)
ATGTTAAGAGCTAAGAATCAGCTTTTTTTACTTTCACCTCATTAC
CTGAGGCAGGTAAAAGAATCATCAGGCTCCAGGCTCATACAGCAA
CGACTTCTACACCAGCAACAGCCCCTTCACCCAGAATGGGCTGCC
CTGGCTAAAAAGCAGCTGAAAGGCAAAAACCCAGAAGACCTAATA
TGGCACACCCCGGAAGGGATCTCTATAAAACCCTTGTATTCCAAG
AGAGATACTATGGACTTACCTGAAGAACTTCCAGGAGTGAAGCCA
TTCACACGTGGACCATATCCTACCATGTATACCTTTAGGCCCTGG
ACCATCCGCCAGTATGCTGGTTTTAGTACTGTGGAAGAAAGCAAT
AAGTTCTATAAGGACAACATTAAGGCTGGTCAGCAGGGATTATCA
GTTGCCTTTGATCTGGCGACACATCGTGGCTATGATTCAGACAAC
CCTCGAGTTCGTGGTGATGTTGGAATGGCTGGAGTTGCTATTGAC
ACTGTGGAAGATACCAAAATTCTTTTTGATGGTATTCCTTTAGAA
AAAATGTCAGTTTCCATGACTATGAATGGAGCAGTTATTCCAGTT
CTTGCAAATTTTATAGTAACTGGAGAAGAACAAGGTGTACCTAAA
GAGAAGCTTACTGGTACCATCCAAAATGATATACTAAAGGAATTT
ATGGTTCGAAATACATACATTTTTCCTCCAGAACCATCCATGAAA
ATTATTGCTGACATATTTGAATATACAGCAAAGCACATGCCAAAA
TTTAATTCAATTTCAATTAGTGGATACCATATGCAGGAAGCAGGG
GCTGATGCCATTCTGGAGCTGGCCTATACTTTAGCAGATGGATTG
GAGTACTCTAGAACTGGACTCCAGGCTGGCCTGACAATTGATGAA
TTTGCACCAAGGTTGTCTTTCTTCTGGGGAATTGGAATGAATTTC
TATATGGAAATAGCAAAGATGAGAGCTGGTAGAAGACTCTGGGCT
CACTTAATAGAGAAAATGTTTCAGCCTAAAAACTCAAAATCTCTT
CTTCTAAGAGCACACTGTCAGACATCTGGATGGTCACTTACTGAG
CAGGATCCCTACAATAATATTGTCCGTACTGCAATAGAAGCAATG
GCAGCAGTATTTGGAGGGACTCAGTCTTTGCACACAAATTCTTTT
GATGAAGCTTTGGGTTTGCCAACTGTGAAAAGTGCTCGAATTGCC
AGGAACACACAAATCATCATTCAAGAAGAATCTGGGATTCCCAAA
GTGGCTGATCCTTGGGGAGGTTCTTACATGATGGAATGTCTCACA
AATGATGTTTATGATGCTGCTTTAAAGCTCATTAATGAAATTGAA
GAAATGGGTGGAATGGCCAAAGCTGTAGCTGAGGGAATACCTAAA
CTTCGAATTGAAGAATGTGCTGCCCGAAGACAAGCTAGAATAGAT
TCTGGTTCTGAAGTAATTGTTGGAGTAAATAAGTACCAGTTGGAA
AAAGAAGACGCTGTAGAAGTTCTGGCAATTGATAATACTTCAGTG
CGAAACAGGCAGATTGAAAAACTTAAGAAGATCAAATCCAGCAGG
GATCAAGCTTTGGCTGAACGTTGTCTTGCTGCACTAACCGAATGT
GCTGCAAGCGGAGATGGAAATATCCTGGCTCTTGCAGTGGATGCA
TCTCGGGCAAGATGTACAGTGGGAGAAATCACAGATGCCCTGAAA
AAGGTATTTGGTGAACATAAAGCGAATGATCGAATGGTGAGTGGA
GCATATCGCCAGGAATTTGGAGAAAGTAAAGAGATAACATCTGCT
ATCAAGAGGGTTCATAAATTCATGGAACGTGAAGGTCGCAGACCT
CGTCTTCTTGTAGCAAAAATGGGACAAGATGGCCATGACAGAGGA
GCAAAAGTTATTGCTACAGGATTTGCTGATCTTGGTTTTGATGTG
GACATAGGCCCTCTTTTCCAGACTCCTCGTGAAGTGGCCCAGCAG
GCTGTGGATGCGGATGTGCATGCTGTGGGCATAAGCACCCTCGCT
GCTGGTCATAAAACCCTAGTTCCTGAACTCATCAAAGAACTTAAC
TCCCTTGGACGGCCAGATATTCTTGTCATGTGTGGAGGGGTGATA
CCACCTCAGGATTATGAATTTCTGTTTGAAGTTGGTGTTTCCAAT
GTATTTGGTCCTGGGACTCGAATCCCAAAGGCTGCCGTTCAGGTG
CTTGATGATATTGAGAAGTGTTTGGAAAAGAAGCAGCAATCTGTA
TAA
Exemplary nucleotide encoding MMUT

In some embodiments, the nucleotide sequence encoding MMUT, or a fragment and/or variant thereof, is codon optimised. An exemplary codon-optimised sequence is provided in SEQ ID NO: 39.

In some embodiments, the nucleotide sequence encoding MMUT, or a fragment and/or variant thereof, comprises or consists of a nucleotide sequence which is at least 70% identical to SEQ ID NO: 39 or a fragment thereof. Suitably, the nucleotide sequence encoding MMUT, or a fragment and/or variant thereof, comprises or consists of a nucleotide sequence which is at least 75%, at least 75%, at least 80%, at least 85%, at least 90%, at least 95%, at least 96%, at least 97%, at least 98% or at least 99% identical to SEQ ID NO: 39 or a fragment thereof.

In some embodiments, the nucleotide sequence encoding MMUT, or a fragment and/or variant thereof, comprises or consists of the nucleotide sequence SEQ ID NO: 39 or a fragment thereof.

(SEQ ID NO: 39)
ATGCTGAGGGCCAAGAACCAGCTGTTCCTGCTGTCCCCACACTAC
CTGCGCCAGGTGAAGGAGAGCTCCGGCTCTAGGCTGATCCAGCAG
CGCCTGCTGCACCAGCAGCAGCCACTGCACCCTGAGTGGGCCGCC
CTGGCCAAGAAGCAGCTGAAGGGCAAGAATCCCGAGGACCTGATC
TGGCACACCCCAGAGGGCATCAGCATCAAGCCCCTGTATTCCAAG
AGGGACACCATGGATCTGCCAGAGGAGCTGCCAGGAGTGAAGCCT
TTCACACGCGGCCCTTACCCAACCATGTATACATTTCGGCCCTGG
ACCATCAGACAGTACGCCGGCTTCAGCACAGTGGAGGAGTCCAAC
AAGTTTTATAAGGACAATATCAAGGCAGGACAGCAGGGCCTGAGC
GTGGCCTTTGATCTGGCCACACACCGCGGCTACGACTCCGATAAC
CCCAGGGTGCGCGGCGACGTGGGAATGGCAGGAGTGGCAATCGAC
ACCGTGGAGGATACAAAGATCCTGTTCGATGGCATCCCTCTGGAG
AAGATGTCCGTGTCTATGACCATGAACGGCGCCGTGATCCCCGTG
CTGGCCAACTTCATCGTGACAGGCGAGGAGCAGGGCGTGCCTAAG
GAGAAGCTGACCGGCACAATCCAGAACGACATCCTGAAGGAGTTC
ATGGTGCGGAATACCTATATCTTTCCCCCTGAGCCATCCATGAAG
ATCATCGCCGACATCTTCGAGTACACAGCCAAGCACATGCCCAAG
TTTAATAGCATCTCCATCTCTGGCTATCACATGCAGGAGGCAGGA
GCAGACGCAATCCTGGAGCTGGCCTACACCCTGGCCGATGGCCTG
GAGTATTCCCGGACCGGCCTGCAGGCAGGCCTGACAATCGACGAG
TTCGCCCCTAGACTGTCTTTCTTTTGGGGCATCGGCATGAACTTT
TACATGGAGATCGCCAAGATGAGGGCAGGCCGGAGACTGTGGGCT
CACCTGATCGAGAAGATGTTCCAGCCTAAGAATAGCAAGTCCCTG
CTGCTGAGAGCCCACTGCCAGACCTCCGGATGGAGCCTGACAGAG
CAGGACCCATATAACAATATCGTGAGAACCGCCATCGAGGCAATG
GCAGCCGTGTTCGGAGGAACCCAGTCTCTGCACACAAACAGCTTT
GATGAGGCCCTGGGCCTGCCTACCGTGAAGAGCGCCCGGATCGCC
AGAAATACACAGATCATCATCCAGGAGGAGTCTGGCATCCCAAAG
GTGGCAGATCCATGGGGAGGAAGCTACATGATGGAGTGCCTGACA
AACGACGTGTATGATGCCGCCCTGAAGCTGATCAATGAGATCGAG
GAGATGGGAGGAATGGCAAAGGCAGTGGCAGAGGGAATCCCAAAG
CTGCGGATCGAGGAGTGTGCAGCAAGGCGCCAGGCAAGAATCGAC
TCTGGCAGCGAAGTGATCGTGGGCGTGAACAAGTACCAGCTGGAG
AAGGAGGACGCCGTGGAGGTGCTGGCCATCGATAACACCTCCGTG
CGGAATAGACAGATCGAGAAGCTGAAGAAGATCAAGTCTAGCCGG
GATCAGGCCCTGGCCGAGAGATGCCTGGCCGCCCTGACAGAGTGT
GCAGCCTCTGGCGACGGAAACATCCTGGCCCTGGCCGTGGATGCC
AGCCGGGCCAGATGTACCGTGGGAGAGATCACAGACGCCCTGAAG
AAGGTGTTCGGCGAGCACAAGGCCAATGATAGGATGGTGAGCGGC
GCCTATCGCCAGGAGTTCGGCGAGTCCAAGGAGATCACCTCTGCC
ATCAAGAGGGTGCACAAGTTTATGGAGAGGGAGGGCAGACGGCCC
AGGCTGCTGGTGGCAAAGATGGGACAGGACGGACACGATAGGGGA
GCCAAAGTGATCGCAACCGGCTTCGCAGACCTGGGATTTGACGTG
GACATCGGCCCTCTGTTCCAGACACCACGGGAGGTGGCACAGCAG
GCAGTGGACGCAGATGTGCACGCAGTGGGCATCAGCACCCTGGCC
GCAGGACACAAGACACTGGTGCCTGAGCTGATCAAGGAGCTGAAC
TCCCTGGGCAGACCAGACATCCTGGTCATGTGCGGCGGCGTGATC
CCACCCCAGGATTACGAGTTCCTGTTTGAAGTGGGCGTGTCTAAC
GTGTTCGGCCCTGGAACCAGGATCCCTAAGGCAGCCGTGCAGGTG
CTGGACGACATCGAGAAGTGTCTGGAGAAGAAGCAGCAGAGCGTG
TGA
Exemplary codon-optimised nucleotide
encoding MMUT

### MMAA

In some embodiments, the MMA-associated polypeptide is MMAA or a fragment and/or variant thereof.

“MMAA” is the abbreviated name of the polypeptide encoded by the MMAA gene and is also known as mitochondrial methylmalonic aciduria type A protein and metabolism of cobalamin associated A. MMAA mediates the transport of cobalamin (Cbl) into mitochondria for the final steps of adenosylcobalamin (AdoCbl) synthesis and assists AdoCbl cofactor delivery from MMAB to the methylmalonyl-CoA mutase (MMUT).

A fragment and/or variant of MMAA may retain MMAA activity. For example, a fragment and/or variant of MMAA may mediate the transport of Cbl into mitochondria and/or assist AdoCbl cofactor delivery from MMAB to MMUT. Suitably, a fragment and/or variant of MMAA may have the same or similar activity to MMAA, e.g. may have at least 40%, at least 50%, at least 60%, at least 70%, at least 80%, at least 90%, at least 95%, or at least 100% of the activity of MMAA. Suitable assays for MMAA activity are known in the art (see e.g. Plessl, T., et al., 2017. Human Mutation, 38(8), pp. 988-1001).

A person skilled in the art would be able to generate fragments and/or variants using conservative substitutions, based on the known structural and functional features of MMAA (see e.g. Plessl, T., et al., 2017. Human Mutation, 38(8), pp. 988-1001 and Froese, D. S., et al., 2010. Journal of Biological Chemistry, 285(49), pp. 38204-38213), and/or based on known variants (see e.g. NCBI Gene ID: 166785 and NCBI HomoloGene: 14586).

The MMAA gene is conserved in chimpanzee, dog, cow, mouse, and rat. The MMAA may be a human MMAA. Suitably, the MMAA may comprise or consist of a polypeptide sequence of UniProtKB accession Q8IVH4, or a fragment and/or variant thereof.

In some embodiments, the MMAA comprises or consists of an amino acid sequence which is at least 70% identical to SEQ ID NO: 40 or a fragment thereof. Suitably, the MMAA comprises or consists of an amino acid sequence which is at least 75%, at least 80%, at least 85%, at least 90%, at least 95%, at least 96%, at least 97%, at least 98% or at least 99% identical to SEQ ID NO: 40 or a fragment thereof.

In some embodiments, the MMAA comprises or consists of SEQ ID NO: 40 or a fragment thereof.

(SEQ ID NO: 40)
MPMLLPHPHQHFLKGLLRAPFRCYHFIFHSSTHLGSGIPCAQPFN
SLGLHCTKWMLLSDGLKRKLCVQTTLKDHTEGLSDKEQRFVDKLY
TGLIQGQRACLAEAITLVESTHSRKKELAQVLLQKVLLYHREQEQ
SNKGKPLAFRVGLSGPPGAGKSTFIEYFGKMLTERGHKLSVLAVD
PSSCTSGGSLLGDKTRMTELSRDMNAYIRPSPTRGTLGGVTRTTN
EAILLCEGAGYDIILIETVGVGQSEFAVADMVDMFVLLLPPAGGD
ELQGIKRGIIEMADLVAVTKSDGDLIVPARRIQAEYVSALKLLRK
RSQVWKPKVIRISARSGEGISEMWDKMKDFQDLMLASGELTAKRR
KQQKVWMWNLIQESVLEHFRTHPTVREQIPLLEQKVLIGALSPGL
AADFLLKAFKSRD
Exemplary MMAA

Suitably, a MMAA variant may comprise Q363H. This variation is considered to be benign based on clinical data.

Suitably, the nucleotide sequence encoding MMAA, or a fragment and/or variant thereof, may comprise or consist of a nucleotide sequence of NCBI reference sequence NM_172250 or NM_001375644, or a fragment and/or variant thereof.

In some embodiments, the nucleotide sequence encoding MMAA, or a fragment and/or variant thereof, comprises or consists of a nucleotide sequence which is at least 70% identical to SEQ ID NO: 41 or a fragment thereof. Suitably, the nucleotide sequence encoding MMAA, or a fragment and/or variant thereof, comprises or consists of a nucleotide sequence which is at least 75%, at least 80%, at least 85%, at least 90%, at least 95%, at least 96%, at least 97%, at least 98% or at least 99% identical to SEQ ID NO: 41 or a fragment thereof.

In some embodiments, the nucleotide sequence encoding MMAA, or a fragment and/or variant thereof, comprises or consists of the nucleotide sequence SEQ ID NO: 41 or a fragment thereof.

(SEQ ID NO: 41)
ATGCCCATGCTGCTACCACATCCTCACCAGCATTTCCTAAAAGGC
CTTTTAAGAGCACCTTTCCGATGTTACCACTTCATCTTTCACTCA
AGTACTCATCTCGGATCAGGAATCCCATGTGCTCAGCCGTTTAAT
TCTCTTGGACTCCATTGTACAAAGTGGATGCTGCTGTCAGATGGC
TTAAAGAGAAAATTATGTGTACAAACAACCTTAAAGGACCACACA
GAAGGACTTTCTGATAAAGAGCAAAGATTTGTGGATAAACTTTAT
ACTGGTTTAATCCAAGGGCAAAGGGCCTGTTTAGCAGAGGCCATA
ACTCTTGTAGAATCAACTCACAGCAGGAAAAAGGAGTTAGCCCAG
GTGCTTCTTCAGAAAGTATTACTTTACCACAGAGAACAAGAACAA
TCAAATAAAGGAAAACCACTAGCATTTCGAGTAGGATTGTCTGGG
CCCCCTGGTGCTGGAAAATCAACATTTATAGAATATTTTGGAAAA
ATGCTTACTGAGAGAGGGCACAAATTATCTGTGCTAGCTGTGGAC
CCTTCTTCTTGTACTAGTGGTGGATCACTCTTAGGTGATAAAACC
CGAATGACTGAGTTATCAAGAGATATGAATGCATACATCAGGCCA
TCTCCTACTAGAGGAACTTTAGGAGGCGTGACAAGGACCACAAAT
GAAGCTATTCTGTTGTGTGAAGGAGCGGGATATGACATAATTCTT
ATTGAAACCGTTGGTGTGGGTCAGTCGGAGTTTGCTGTTGCTGAC
ATGGTTGACATGTTTGTTTTACTACTGCCACCAGCAGGAGGAGAT
GAGCTGCAGGGTATCAAAAGGGGTATAATCGAGATGGCAGATCTG
GTAGCTGTAACTAAATCTGATGGAGACTTGATTGTGCCAGCTCGA
AGGATACAAGCGGAATATGTGAGTGCACTGAAATTACTCCGCAAA
CGTTCACAAGTCTGGAAACCAAAGGTAATTCGTATTTCTGCCCGA
AGTGGAGAGGGGATCTCTGAAATGTGGGATAAAATGAAAGATTTC
CAGGACCTAATGCTTGCCAGTGGGGAGCTGACTGCCAAACGACGG
AAGCAACAGAAAGTTTGGATGTGGAATCTCATTCAGGAAAGTGTG
TTAGAGCATTTCAGGACCCACCCCACAGTCCGGGAACAGATTCCA
CTTCTGGAACAAAAGGTTCTCATTGGGGCCCTGTCCCCAGGACTA
GCAGCAGACTTCTTGTTAAAAGCTTTTAAAAGCAGAGACTAA
Exemplary nucleotide encoding MMAA

### MMAB

In some embodiments, the MMA-associated polypeptide is MMAB or a fragment and/or variant thereof.

“MMAB” is the abbreviated name of the polypeptide encoded by the MMAB gene and is also known as mitochondrial methylmalonic aciduria type B protein and corrinoid adenosyltransferase. MMAB catalyses the final step in the conversion of vitamin B12 into adenosylcobalamin (AdoCbl) and may also deliver AdoCbl to MMUT.

A fragment and/or variant of MMAB may retain MMAB activity (see e.g. EC 2.5.1.17). For example, a fragment and/or variant of MMAB may catalyse the final step in the conversion of vitamin B12 into AdoCbl and/or deliver AdoCbl to MMUT. Suitably, a fragment and/or variant of MMAB may have the same or similar activity to MMAB, e.g. may have at least 40%, at least 50%, at least 60%, at least 70%, at least 80%, at least 90%, at least 95%, or at least 100% of the activity of MMAB. Suitable assays for MMAB activity are known in the art (see e.g. Forny, P., et al., 2021. Human genetics, pp.1-15)

A person skilled in the art would be able to generate fragments and/or variants using conservative substitutions, based on the known structural and functional features of MMAB (see e.g. Forny, P., et al., 2021. Human genetics, pp. 1-15; and Schubert, H. L. and Hill, C. P., 2006. Biochemistry, 45(51), pp. 15188-15196) and/or based on known variants (see e.g. NCBI Gene ID: 326625 and NCBI HomoloGene: 12680). Suitably, a fragment of MMAB and/or a MMAB variant comprises a cobalamin adenosyltransferase domain.

The MMAB gene is conserved in chimpanzee, Rhesus monkey, dog, cow, mouse, and rat. The MMAB may be a human MMAB. Suitably, the MMAB may comprise or consist of a polypeptide sequence of UniProtKB accession Q96EY8, or a fragment and/or variant thereof.

In some embodiments, the MMAB comprises or consists of an amino acid sequence which is at least 70% identical to SEQ ID NO: 42 or a fragment thereof. Suitably, the MMAB comprises or consists of an amino acid sequence which is at least 75%, at least 80%, at least 85%, at least 90%, at least 95%, at least 96%, at least 97%, at least 98% or at least 99% identical to SEQ ID NO: 42 or a fragment thereof.

In some embodiments, the MMAB comprises or consists of SEQ ID NO: 42 or a fragment thereof.

(SEQ ID NO: 42)
MAVCGLGSRLGLGSRLGLRGCFGAARLLYPRFQSRGPQGVEDGDR
PQPSSKTPRIPKIYTKTGDKGESSTFTGERRPKDDQVFEAVGTTD
ELSSAIGFALELVTEKGHTFAEELQKIQCTLQDVGSALATPCSSA
REAHLKYTTFKAGPILELEQWIDKYTSQLPPLTAFILPSGGKISS
ALHFCRAVCRRAERRVVPLVQMGETDANVAKFLNRLSDYLFTLAR
YAAMKEGNQEKIYMKNDPSAESEGL
Exemplary MMAB

Suitably, a MMAB variant may comprise one or more variation selected from C4R, R19H, R19Q, S52N, T62M, S69N, T222M, and M239K. These are considered to be benign (or likely benign) variations based on clinical data.

Suitably, the nucleotide sequence encoding MMAB, or a fragment and/or variant thereof, may comprise or consist of a nucleotide sequence of NCBI reference sequence NM_052845, or a fragment and/or variant thereof.

In some embodiments, the nucleotide sequence encoding MMAB, or a fragment and/or variant thereof, comprises or consists of a nucleotide sequence which is at least 70% identical to SEQ ID NO: 43 or a fragment thereof. Suitably, the nucleotide sequence encoding MMAB, or a fragment and/or variant thereof, comprises or consists of a nucleotide sequence which is at least 75%, at least 80%, at least 85%, at least 90%, at least 95%, at least 96%, at least 97%, at least 98% or at least 99% identical to SEQ ID NO: 43 or a fragment thereof.

In some embodiments, the nucleotide sequence encoding MMAB, or a fragment and/or variant thereof, comprises or consists of the nucleotide sequence SEQ ID NO: 43 or a fragment thereof.

(SEQ ID NO: 43)
ATGGCTGTGTGCGGCCTGGGGAGCCGTCTTGGCCTGGGGAGCCGT
CTTGGCCTGCGCGGGTGCTTCGGCGCCGCCAGGCTCCTGTATCCC
CGTTTCCAGAGCCGCGGCCCTCAGGGCGTGGAAGACGGGGACAGG
CCACAGCCTTCCTCGAAGACACCCAGGATCCCCAAGATTTACACC
AAAACGGGAGACAAAGGGTTTTCTAGTACCTTCACAGGAGAAAGG
AGACCCAAAGATGACCAAGTGTTTGAAGCCGTGGGAACTACAGAT
GAATTAAGTTCAGCTATTGGGTTTGCTCTGGAATTAGTCACAGAA
AAGGGCCATACATTTGCCGAAGAGCTTCAGAAAATCCAGTGCACA
TTGCAGGACGTCGGCTCGGCCCTGGCGACACCATGCTCCTCGGCC
CGGGAGGCTCACTTAAAGTATACCACGTTCAAGGCGGGGCCCATC
CTGGAGCTGGAGCAGTGGATCGACAAGTACACCAGCCAGCTCCCA
CCACTCACGGCCTTCATCCTGCCTTCGGGAGGCAAGATCAGCTCG
GCGCTGCATTTCTGCCGGGCCGTGTGCCGCCGGGCCGAGAGACGT
GTGGTGCCTCTTGTCCAGATGGGAGAGACCGATGCGAACGTGGCC
AAGTTCTTAAACAGACTCAGTGACTATCTCTTCACGCTAGCCAGA
TATGCAGCCATGAAGGAGGGGAATCAAGAGAAAATATACATGAAA
AATGACCCATCGGCCGAGTCTGAGGGACTCTGA
Exemplary nucleotide encoding MMAB

### MMADHC

In some embodiments, the MMA-associated polypeptide is MMADHC or a fragment and/or variant thereof.

“MMADHC” is the abbreviated name of the polypeptide encoded by the MMADHC gene and is also known as cobalamin trafficking protein CblD and mitochondrial methylmalonic aciduria and homocystinuria type D protein. MMADHC is involved in cobalamin metabolism and trafficking and plays important roles in processing and targeting the Cbl cofactor to its destination enzymes. It has been suggested that MMACHC-bound Cbl can partner with MMADHC to be ferried to methylmalonyl-CoA mutase (see e.g. Froese, D. S., et al., 2015. Journal of Biological Chemistry, 290(49), pp. 29167-29177).

A fragment and/or variant of MMADHC may retain MMADHC activity. For example, a fragment and/or variant of MMADHC may traffick Cbl to methylmalonyl-CoA mutase. Suitably, a fragment and/or variant of MMADHC may have the same or similar activity to MMADHC, e.g. may have at least 40%, at least 50%, at least 60%, at least 70%, at least 80%, at least 90%, at least 95%, or at least 100% of the activity of MMADHC.

A person skilled in the art would be able to generate fragments and/or variants using conservative substitutions, based on the known structural and functional features of MMADHC (see e.g. Froese, D. S., et al., 2015. Journal of Biological Chemistry, 290(49), pp. 29167-29177; Jusufi, J., et al., 2014. Journal of inherited metabolic disease, 37(5), pp. 841-849; Deme, J. C., et al., 2012. Molecular genetics and metabolism, 107(3), pp. 352-362; and Yamada, K., et al., 2015. Journal of Biological Chemistry, 290(49), pp. 29155-29166), and/or based on known variants (see e.g. NCBI Gene ID: 27249 and NCBI HomoloGene: 9248).

The MMADHC gene is conserved in chimpanzee, Rhesus monkey, dog, cow, mouse, and rat. The MMADHC may be a human MMADHC. Suitably, the MMADHC may comprise or consist of a polypeptide sequence of UniProtKB accession Q9H3L0, or a fragment and/or variant thereof.

In some embodiments, the MMADHC comprises or consists of an amino acid sequence which is at least 70% identical to SEQ ID NO: 44 or a fragment thereof. Suitably, the MMADHC comprises or consists of an amino acid sequence which is at least 75%, at least 80%, at least 85%, at least 90%, at least 95%, at least 96%, at least 97%, at least 98% or at least 99% identical to SEQ ID NO: 44 or a fragment thereof.

In some embodiments, the MMADHC comprises or consists of SEQ ID NO: 44 or a fragment thereof.

(SEQ ID NO: 44)
MANVLCNRARLVSYLPGFCSLVKRVVNPKAFSTAGSSGSDESHVA
AAPPDICSRTVWPDETMGPFGPQDQRFQLPGNIGFDCHLNGTASQ
KKSLVHKTLPDVLAEPLSSERHEFVMAQYVNEFQGNDAPVEQEIN
SAETYFESARVECAIQTCPELLRKDFESLFPEVANGKLMILTVTQ
KTKNDMTVWSEEVEIEREVLLEKFINGAKEICYALRAEGYWADFI
DPSSGLAFFGPYTNNTLFETDERYRHLGFSVDDLGCCKVIRHSLW
GTHVVVGSIFTNATPDSHIMKKLSGN
Exemplary MMADHC

Suitably, a MMADHC variant may comprise one or more variation selected from K29N, E138K, S1431, R158Q, N206S, and P236L. These are considered to be benign (or likely benign) variations based on clinical data.

Suitably, the nucleotide sequence encoding MMADHC, or a fragment and/or variant thereof, may comprise or consist of a nucleotide sequence of NCBI reference sequence NM_015702, or a fragment and/or variant thereof.

In some embodiments, the nucleotide sequence encoding MMADHC, or a fragment and/or variant thereof, comprises or consists of a nucleotide sequence which is at least 70% identical to SEQ ID NO: 45 or a fragment thereof. Suitably, the nucleotide sequence encoding MMADHC, or a fragment and/or variant thereof, comprises or consists of a nucleotide sequence which is at least 75%, at least 80%, at least 85%, at least 90%, at least 95%, at least 96%, at least 97%, at least 98% or at least 99% identical to SEQ ID NO: 45 or a fragment thereof.

In some embodiments, the nucleotide sequence encoding MMADHC, or a fragment and/or variant thereof, comprises or consists of the nucleotide sequence SEQ ID NO: 45 or a fragment thereof.

(SEQ ID NO: 45)
ATGGCCAATGTGCTTTGTAACAGAGCCAGACTGGTTTCCTATCTC
CCAGGATTTTGCTCTTTAGTTAAAAGGGTTGTCAATCCCAAAGCC
TTTTCGACTGCAGGATCATCAGGTTCGGATGAGTCTCATGTGGCT
GCTGCACCTCCAGATATATGCTCTCGAACAGTGTGGCCTGATGAA
ACTATGGGACCCTTTGGACCTCAAGATCAGAGGTTCCAGCTTCCT
GGGAACATAGGTTTTGATTGTCACCTCAATGGGACTGCTTCACAG
AAGAAAAGCCTGGTTCATAAAACTTTGCCTGATGTTCTAGCAGAA
CCTTTATCAAGTGAAAGACATGAGTTTGTGATGGCACAATATGTG
AATGAATTTCAGGGTAATGATGCACCTGTTGAACAAGAAATTAAC
AGTGCAGAAACTTACTTTGAAAGTGCCAGAGTAGAGTGTGCAATA
CAGACATGTCCAGAATTGCTGCGAAAAGATTTTGAATCACTGTTT
CCAGAAGTAGCTAATGGCAAACTAATGATTCTGACTGTAACACAA
AAAACTAAGAATGATATGACTGTTTGGAGTGAAGAAGTAGAAATT
GAAAGAGAAGTGCTCTTAGAAAAGTTCATCAATGGTGCTAAGGAA
ATTTGCTATGCTCTTCGAGCTGAGGGTTATTGGGCTGACTTTATT
GACCCATCATCTGGTTTGGCATTTTTTGGACCATATACAAACAAC
ACTCTTTTTGAAACTGATGAACGCTACCGACATTTAGGATTCTCT
GTTGATGACCTTGGATGCTGTAAAGTGATTCGTCATAGTCTCTGG
GGTACCCATGTAGTTGTAGGGAGTATCTTCACTAATGCAACACCA
GACAGCCATATTATGAAGAAATTAAGTGGAAATTAG
Exemplary nucleotide encoding MMADHC

### MCEE

In some embodiments, the MMA-associated polypeptide is MCEE or a fragment and/or variant thereof.

“MCEE” is the abbreviated name of the polypeptide encoded by the MCEE gene and is also known as mitochondrial methylmalonyl-CoA epimerase and methylmalonyl-CoA racemase. MCEE interconverts D- and L-methylmalonyl-CoA during the degradation of branched-chain amino acids, odd chain-length fatty acids, and other metabolites.

A fragment and/or variant of MCEE may retain MCEE activity (see e.g. EC 5.1.99.1). For example, a fragment and/or variant of MCEE may convert (S)-methylmalonyl-CoA to (R)-methylmalonyl-CoA. Suitably, a fragment and/or variant of MCEE may have the same or similar activity to MCEE, e.g. may have at least 40%, at least 50%, at least 60%, at least 70%, at least 80%, at least 90%, at least 95%, or at least 100% of the activity of MCEE. Suitable assays for MCEE activity are known in the art (see e.g. Heuberger, K., et al., 2019. BBA-Molecular Basis of Disease, 1865(6), pp.1265-1272)

A person skilled in the art would be able to generate fragments and/or variants using conservative substitutions, based on the known structural and functional features of MCEE (see e.g. Heuberger, K., et al., 2019. BBA-Molecular Basis of Disease, 1865(6), pp. 1265-1272), and/or based on known variants (see e.g. NCBI Gene ID: 84693 and NCBI HomoloGene: 13078).

The MCEE gene is conserved in chimpanzee, Rhesus monkey, dog, cow, mouse, and rat. The MCEE may be a human MCEE. Suitably, the MCEE may comprise or consist of a polypeptide sequence of UniProtKB accession Q96PE7, or a fragment and/or variant thereof.

In some embodiments, the MCEE comprises or consists of an amino acid sequence which is at least 70% identical to SEQ ID NO: 46 or a fragment thereof. Suitably, the MCEE comprises or consists of an amino acid sequence which is at least 75%, at least 80%, at least 85%, at least 90%, at least 95%, at least 96%, at least 97%, at least 98% or at least 99% identical to SEQ ID NO: 46 or a fragment thereof.

In some embodiments, the MCEE comprises or consists of SEQ ID NO: 46 or a fragment thereof.

(SEQ ID NO: 46)
MARVLKAAAANAVGLFSRLQAPIPTVRASSTSQPLDQVTGSVWNL
GRLNHVAIAVPDLEKAAAFYKNILGAQVSEAVPLPEHGVSVVFVN
LGNTKMELLHPLGRDSPIAGFLQKNKAGGMHHICIEVDNINAAVM
DLKKKKIRSLSEEVKIGAHGKPVIFLHPKDCGGVLVELEQA
Exemplary MCEE

Suitably, a MCEE variant may comprise one or more variation selected from A76V, R104L, and R143H. These are considered to be benign (or likely benign) variations based on clinical data.

Suitably, the nucleotide sequence encoding MCEE, or a fragment and/or variant thereof, may comprise or consist of a nucleotide sequence of NCBI reference sequence NM_032601, or a fragment and/or variant thereof.

In some embodiments, the nucleotide sequence encoding MCEE, or a fragment and/or variant thereof, comprises or consists of a nucleotide sequence which is at least 70% identical to SEQ ID NO: 47 or a fragment thereof. Suitably, the nucleotide sequence encoding MCEE, or a fragment and/or variant thereof, comprises or consists of a nucleotide sequence which is at least 75%, at least 80%, at least 85%, at least 90%, at least 95%, at least 96%, at least 97%, at least 98% or at least 99% identical to SEQ ID NO: 47 or a fragment thereof.

In some embodiments, the nucleotide sequence encoding MCEE, or a fragment and/or variant thereof, comprises or consists of the nucleotide sequence SEQ ID NO: 47 or a fragment thereof.

(SEQ ID NO: 47)
ATGGCGCGGGTGCTGAAGGCTGCAGCCGCGAATGCCGTAGGGCTT
TTTTCCAGACTTCAAGCTCCCATTCCAACAGTAAGAGCTTCTTCC
ACATCACAGCCCTTGGATCAAGTGACAGGTTCTGTGTGGAACCTG
GGTCGACTCAACCATGTAGCCATAGCAGTGCCAGATTTGGAAAAG
GCTGCAGCATTTTATAAGAATATTCTGGGGGCCCAGGTAAGTGAA
GCGGTCCCTCTTCCTGAACATGGAGTATCTGTTGTTTTTGTCAAC
CTGGGAAATACCAAGATGGAACTGCTTCATCCATTGGGACGTGAC
AGTCCAATTGCAGGTTTTCTGCAGAAAAACAAGGCTGGAGGAATG
CATCACATCTGCATCGAGGTGGATAATATTAATGCAGCTGTGATG
GATTTGAAAAAAAAGAAGATCCGCAGTCTAAGTGAAGAGGTCAAA
ATAGGAGCACATGGAAAACCAGTGATTTTTCTCCATCCTAAAGAC
TGTGGTGGAGTCCTTGTGGAACTGGAGCAAGCTTGA
Exemplary nucleotide encoding MCEE

### Regulatory Elements

The lentiviral vector of the present invention may further comprise one or more regulatory elements which may act pre- or post-transcriptionally. Suitably, the protein-coding sequence is operably linked to one or more regulatory elements which may act pre- or post-transcriptionally. The one or more regulatory elements may facilitate expression of the transgene in liver cells (e.g. hepatocytes).

As used herein, a “regulatory element” may refer any nucleotide sequence that facilitates expression of a polypeptide, e.g. acts to increase expression of a transcript or to enhance mRNA stability. Suitable regulatory elements include for example promoters, enhancer elements, post-transcriptional regulatory elements, polyadenylation sites, and Kozak sequences.

### Promoter

The lentiviral vector of the present invention may comprise a promoter, preferably a liver-specific (e.g. hepatocyte-specific) promoter. Suitably, the protein-coding sequence is operably linked to a promoter, preferably a liver-specific (e.g. hepatocyte-specific) promoter.

A “promoter” may refer to a region of DNA that leads to initiation of transcription of a gene. Promoters are located near the transcription start sites of genes, upstream on the DNA (towards the 5′ region of the sense strand).

As used herein, a “tissue-specific promoter” may refer to a promoter which preferentially facilitates expression of a transgene in a specific type of cells or tissue. Suitably, a tissue-specific promoter may facilitate higher expression of a transgene in one cell-type as compared to other cell-types. Higher expression may be measured for example by measuring the expression of a transgene, e.g. green fluorescence protein (GFP), operably linked to the promoter, wherein expression of the transgene correlates with the ability of the promoter to facilitate expression of a gene. For example, a tissue-specific promoter may be a promoter which facilitates transgene expression levels at least 10% higher, at least 20% higher, at least 30% higher, at least 40% higher, at least 50% higher, at least 100% higher, at least 200% higher, at least 300% higher, at least 400% higher, at least 500% higher, or at least 1000% higher in one cell-type as compared to expression levels in other cell-types.

In some embodiments, the promoter is a liver-specific promoter. In some embodiments, the promoter is a hepatocyte-specific promoter.

Suitably, the promoter may be (or may be derived from) a promoter associated with a gene with selective expression in human liver cells (e.g. hepatocytes). Suitably, the promoter may be (or may be derived from) a promoter associated with a gene with selective expression in human hepatocyte cells. Methods to identify promoters associated with genes will be well known to those of skill in the art.

Exemplary liver-specific and/or hepatocyte-specific promoters are described in Kattenhorn, L. M., et al., 2016. Human gene therapy, 27(12), pp. 947-961 and include transthyretin (TTR) promoters, alpha-1-antityrpsin (AAT) promoters, thyroxine-binding globulin (TBG) promoters, APoE/hAAT promoters, HCR-hAAT promoters, LP1 promoters, and HLP promoters.

An engineered promoter variant derived from any of these promoters may be used, provided that the variant retains the capacity to drive liver-specific and/or hepatocyte-specific expression of a transgene which is operably coupled to the promoter. A skilled person will be arrive at such variants using methods known in the art. The variant may have at least 70%, at least 80%, at least 90%, at least 95%, at least 96%, at least 97%, at least 98% or at least 99% identity to any of the promoters.

A fragment of any of these promoters (or variants thereof) may be used, provided that the fragment retains the capacity to drive liver-specific and/or hepatocyte-specific expression of a transgene which is operably coupled to the promoter. A skilled person will be able to arrive at such fragments using methods known in the art. The fragment may be, for example, at least 200 nucleotides, at least 300 nucleotides, at least 400 nucleotides, at least 500 nucleotides, or at least 1000 nucleotides in length.

In some embodiments, the promoter is selected from the group consisting of: a transthyretin (TTR) promoter, an alpha-1-antityrpsin (AAT) promoter, a thyroxine-binding globulin (TBG) promoter, an APoE/hAAT promoter, a HCR-hAAT promoter, a LP1 promoter, and a HLP promoter.

In some embodiments, the promoter is a TTR promoter, or a variant and/or fragment thereof. In some embodiments, the promoter is an Enh1mTTR (ET) promoter, or a variant and/or fragment thereof.

An exemplary ET promoter is provided in GenBank accession number AY661265. In some embodiments, the ET promoter comprises or consists of a nucleotide sequence which is at least 70% identical to SEQ ID NO: 19 or a fragment thereof. Suitably, the ET promoter comprises or consists of a nucleotide sequence which is at least 75%, at least 80%, at least 85%, at least 90%, at least 95%, at least 96%, at least 97%, at least 98%, or at least 99% identical to SEQ ID NO: 19 or a fragment thereof.

In some embodiments, the ET promoter comprises or consists of the nucleotide sequence SEQ ID NO: 19 or a fragment thereof.

(SEQ ID NO: 19)
CGCGAGTTAATAATTACCAGCGCGGGCCAAATAAATAATCCGCGA
GGGGCAGGTGACGTTTGCCCAGCGCGCGCTGGTAATTATTAACCT
CGCGAATATTGATTCGAGGCCGCGATTGCCGCAATCGCGAGGGGC
AGGTGACCTTTGCCCAGCGCGCGTTCGCCCCGCCCCGGACGGTAT
CGATAAGCTTAGGAGCTTGGGCTGCAGGTCGAGGGCACTGGGAGG
ATGTTGAGTAAGATGGAAAACTACTGATGACCCTTGCAGAGACAG
AGTATTAGGACATGTTTGAACAGGGGCCGGGCGATCAGCAGGTAG
CTCTAGAGGATCCCCGTCTGTCTGCACATTTCGTAGAGCGAGTGT
TCCGATACTCTAATCTCCCTAGGCAAGGTTCATATTTGTGTAGGT
TACTTATTCTCCTTTTGTTGACTAAGTCAATAATCAGAATCAGCA
GGTTTGGAGTCAGCTTGGCAGGGATCAGCAGCCTGGGTTGGAAGG
AGGGGGTATAAAAGCCCCTTCACCAGGAGAAGCCGTCACACAGAT
CCACAAGCTCCTG
Exemplary ET promoter

In some embodiments, the promoter is an AAT promoter, or a variant and/or fragment thereof. In some embodiments, the promoter is a human AAT (hAAT) promoter, or a variant and/or fragment thereof.

In some embodiments, the hAAT promoter comprises or consists of a nucleotide sequence which is at least 70% identical to SEQ ID NO: 20 or a fragment thereof. Suitably, the hAAT promoter comprises or consists of a nucleotide sequence which is at least 75%, at least 80%, at least 85%, at least 90%, at least 95%, at least 96%, at least 97%, at least 98%, or at least 99% identical to SEQ ID NO: 20 or a fragment thereof.

In some embodiments, the hAAT promoter comprises or consists of the nucleotide sequence SEQ ID NO: 20 or a fragment thereof.

(SEQ ID NO: 20)
GATCTTGCTACCAGTGGAACAGCCACTAAGGATTCTGCAGTGAGA
GCAGAGGGCCAGCTAAGTGGTACTCTCCCAGAGACTGTCTGACTC
ACGCCACCCCCTCCACCTTGGACACAGGACGCTGTGGTTTCTGAG
CCAGGTACAATGACTCCTTTCGGTAAGTGCAGTGGAAGCTGTACA
CTGCCCAGGCAAAGCGTCCGGGCAGCGTAGGCGGGCGACTCAGAT
CCCAGCCAGTGGACTTAGCCCCTGTTTGCTCCTCCGATAACTGGG
GTGACCTTGGTTAATATTCACCAGCAGCCTCCCCCGTTGCCCCTC
TGGATCCACTGCTTAAATACGGACGAGGACAG
Exemplary hAAT promoter

The promoter may be a constitutive promoter. As used herein, a “constitutive promoter” is a promoter which is always active.

Alternatively, the promoter may be an inducible promoter. As used herein, an “inducible promoter” is a promoter which is only active under specific conditions. For example, expression of the transgene may be induced by a small molecule or drug (e.g. which binds to a promoter, regulatory sequence or to a transcriptional repressor or activator molecule) or by using an environmental trigger. Types of inducible promoter include chemically-inducible promoters (e.g. a Tet-on system); temperature-inducible promoters (e.g. Hsp70 or Hsp90-derived promoters); and light-inducible promoters. Suitably, the promoter is chemically-inducible. Any suitable method for engineering an inducible promoter may be used.

### Enhancer Elements

The lentiviral vector of the present invention may comprise an enhancer, preferably a liver-specific (e.g. hepatocyte-specific) enhancer. Suitably, the protein-coding sequence is operably linked to an enhancer, preferably a liver-specific (e.g. hepatocyte-specific) enhancer.

An “enhancer” or “enhancer element” may refer a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site.

As used herein, a “tissue-specific enhancer” is an enhancer which preferentially facilitates expression of a gene in specific cells or tissues. Suitably, a tissue-specific enhancer may facilitate higher expression of a gene in specific cells-types as compared to other cell-types. Higher expression may be measured for example by measuring the expression of a transgene, e.g. green fluorescence protein (GFP), operably linked to the enhancer, wherein expression of the transgene correlates with the ability of the enhancer to facilitate expression of a gene. For example, a tissue-specific enhancer may be an enhancer which facilitates gene expression levels at least 10% higher, at least 20% higher, at least 30% higher, at least 40% higher, at least 50% higher, at least 100% higher, at least 200% higher, at least 300% higher, at least 400% higher, at least 500% higher, or at least 1000% higher in a specific cell-type compared to expression levels in other cell types.

Suitable tissue-specific enhancers will be well known to those of skill in the art. The enhancer may be a liver-specific enhancer, preferably a hepatocyte-specific enhancer.

Suitably, the enhancer may be (or may be derived from) an enhancer associated with a gene with selective expression in human liver cells (e.g. hepatocytes). Suitably, the enhancer may be (or may be derived from) an enhancer associated with a gene with selective expression in human hepatocyte cells. Methods to identify the enhancer regions associated with genes will be well known to those of skill in the art.

Exemplary liver-specific and/or hepatocyte-specific enhancers are described in Kramer, M. G., et al., 2003. Molecular therapy, 7(3), pp. 375-385, and include enhancer regions of the albumin, α1-antitrypsin, hepatitis B virus core protein, and hemopexin genes. Other liver-specific and/or hepatocyte-specific enhancers include apolipoprotein E (APoE) enhancers, hepatic control region (HCR) enhancers, and alpha-1-antitrypsin (AAT) enhancers.

An engineered enhancer variant derived from any of these enhancers may be used, provided that the variant retains the capacity to drive liver-specific and/or hepatocyte-specific expression of a transgene which is operably coupled to the enhancer. A skilled person will be arrive at such variants using methods known in the art. The variant may have at least 70%, at least 80%, at least 90%, at least 95%, at least 96%, at least 97%, at least 98% or at least 99% identity to any of the enhancers.

A fragment of any of these enhancers (or variants thereof) may be used, provided that the fragment retains the capacity to drive liver-specific and/or hepatocyte-specific expression of a transgene which is operably coupled to the enhancer. A skilled person will be able to arrive at such fragments using methods known in the art. The fragment may be at least 200 nucleotides, at least 300 nucleotides, at least 400 nucleotides, at least 500 nucleotides, or at least 1000 nucleotides in length.

The vector of the present invention may comprise a liver-specific promoter and/or a liver-specific enhancer, i.e. a liver-specific promoter and/or enhancer. Suitably, the protein-coding sequence is operably linked to a liver-specific promoter and/or enhancer. Suitably, the protein-coding sequence is operably linked to a hepatocyte-specific promoter and/or enhancer. The promoter and enhancer may be a combination of any of the above, for example a hAAT promoter and an ApoE or HCR enhancer.

### Post-Transcriptional Regulatory Elements

The lentiviral vector of the present invention may comprise one or more further post-transcriptional regulatory elements (e.g. in addition to one or more miRNA target sequence). Suitably, the protein-coding sequence is operably linked to one or more further post-transcriptional regulatory elements. The further post-transcriptional regulatory element may improve gene expression.

The lentiviral vector of the present invention may comprise a Woodchuck Hepatitis Virus Post-transcriptional Regulatory Element (WPRE). Suitably, the protein-coding sequence is operably linked to a WPRE.

Suitable WPRE sequences will be well known to those of skill in the art (see e.g. Zufferey, R., et al., 1999. Journal of virology, 73(4), pp. 2886-2892; and Zanta-Boussif, M. A. et al., 2009. Gene therapy, 16(5), pp. 605-619). Suitably, the WPRE is a wild-type WPRE or is a mutant WPRE. For example, the WPRE may be mutated to abrogate translation of the woodchuck hepatitis virus X protein (WHX) e.g. by mutating the WHX ORF translation start codon.

In some embodiments, the WPRE comprises or consists of a nucleotide sequence which is at least 70% identical to SEQ ID NO: 21 or a fragment thereof. Suitably, the WPRE comprises or consists of a nucleotide sequence which is at least 75%, at least 80%, at least 85%, at least 90%, at least 95%, at least 96%, at least 97%, at least 98%, or at least 99% identical to SEQ ID NO: 21 or a fragment thereof.

In some embodiments, the WPRE comprises or consists of the nucleotide sequence SEQ ID NO: 21 or a fragment thereof.

(SEQ ID NO: 21)
AATCAACCTCTGGATTACAAAATTTGTGAAAGATTGACTGGTATT
CTTAACTATGTTGCTCCTTTTACGCTATGTGGATACGCTGCTTTA
ATGCCTTTGTATCATGCTATTGCTTCCCGTATGGCTTTCATTTTC
TCCTCCTTGTATAAATCCTGGTTGCTGTCTCTTTATGAGGAGTTG
TGGCCCGTTGTCAGGCAACGTGGCGTGGTGTGCACTGTGTTTGCT
GACGCAACCCCCACTGGTTGGGGCATTGCCACCACCTGTCAGCTC
CTTTCCGGGACTTTCGCTTTCCCCCTCCCTATTGCCACGGCGGAA
CTCATCGCCGCCTGCCTTGCCCGCTGCTGGACAGGGGCTCGGCTG
TTGGGCACTGACAATTCCGTGGTGTTGTCGGGGAAATCATCGTCC
TTTCCTTGGCTGCTCGCCTGTGTTGCCACCTGGATTCTGCGCGGG
ACGTCCTTCTGCTACGTCCCTTCGGCCCTCAATCCAGCGGACCTT
CCTTCCCGC
Exemplary WPRE

### Polyadenylation Sequence

The lentiviral vector of the present invention may comprise a polyadenylation sequence. Suitably, the protein-coding sequence is operably linked to a polyadenylation sequence. A polyadenylation sequence may be inserted after the protein-coding sequence to improve transgene expression.

A polyadenylation sequence typically comprises a polyadenylation signal, a polyadenylation site and a downstream element: the polyadenylation signal comprises the sequence motif recognised by the RNA cleavage complex; the polyadenylation site is the site of cleavage at which a poly-A tails is added to the mRNA; the downstream element is a GT-rich region which usually lies just downstream of the polyadenylation site, which is important for efficient processing.

Suitable polyadenylation sequences will be well known to those of skill in the art (see e.g. Schambach, A., et al., 2007. Molecular Therapy, 15(6), pp. 1167-1173; and Choi, J. H. et al., 2014. Molecular brain, 7(1), pp. 1-10). Exemplary polyadenylation sequences include the bGH poly(A) signal sequence and SV40pA signal sequence.

Suitably, the polyadenylation sequence may be present in the 3′ LTR (i.e. the lentiviral vector does not comprise an additional polyadenylation sequence).

### Kozak Sequence

The lentiviral vector of the present invention may comprise a Kozak sequence. Suitably, the protein-coding sequence is operably linked to a Kozak sequence. A Kozak sequence may be inserted before the start codon to improve the initiation of translation.

Suitable Kozak sequences will be well known to those of skill in the art (see e.g. Kozak, M., 1987. Nucleic acids research, 15(20), pp. 8125-8148).

In some embodiments, the Kozak sequence comprises or consists of a nucleotide sequence which is at least 80% identical to SEQ ID NO: 22 or a fragment thereof.

In some embodiments, the Kozak sequence comprises or consists of the nucleotide sequence SEQ ID NO: 22 or a fragment thereof.

| | Exemplary Kozak sequence |
|---|---|
| (SEQ ID NO: 22) | |
| | GCCACC |

### Other Cis-Acting Elements

The lentiviral vector of the present invention may comprise any other suitable cis-acting elements, such as one or more of a rev response element (RRE); a retroviral psi packaging element; a primer binding site (PBS); a TAT activation region (TAR); splice donor and acceptor sites; and central and terminal polypurine tracts.

### Long Terminal Repeats (LTRs)

The lentiviral vector of the present invention may comprise one or more long terminal repeat (LTR). As described above, LTRs are responsible for proviral integration and transcription. Typically, a naturally occurring LTR comprises U3, R, and U5 regions.

The lentiviral vector may comprise a 5′ LTR and/or a 3′ LTR. The lentiviral vector may comprise a 5′ LTR and a 3′ LTR. Suitably, a 5′ LTR comprises R and U5 regions, and optionally comprise a U3 region. Suitably, a 3′ LTR comprises U3, R, and U5 regions.

Suitable LTR sequences will be well known to those of skill in the art (see e.g. Frech, K., et al., 1996. Virology, 224(1), pp. 256-267).

In some embodiments, a LTR comprises or consists of a nucleotide sequence which is at least 70% identical to SEQ ID NO: 23 or a fragment thereof. Suitably, a LTR comprises or consists of a nucleotide sequence which is at least 75%, at least 80%, at least 85%, at least 90%, at least 95%, at least 96%, at least 97%, at least 98%, or at least 99% identical to SEQ ID NO: 23 or a fragment thereof.

In some embodiments, a LTR comprises or consists of the nucleotide sequence SEQ ID NO: 23 or a fragment thereof.

Exemplary LTR
(SEQ ID NO: 23)
TGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTC
AAGTAGTGTGTGCCCGTCTGTTGTGTGACTCTGGTAACTAGAGATCCCTC
AGACCCTTTTAGTCAGTGTGGAAAATCTCTAGCAG

The lentiviral vector of the present invention may comprise one or more self-inactivating long terminal repeat (SIN-LTR). A “SIN-LTR” may comprise a deletion that abolishes transcription of the full-length virus after it has incorporated into a host cell. For example, a 3′ SIN-LTR may comprise a deletion in the U3 region removing the promoter/enhancer elements (see e.g. Zufferey, R., et al., 1998. Journal of virology, 72(12), pp. 9873-9880). This deletion is copied into the 5′ LTR after reverse transcription, thereby making the gene expression in target cells dependent on an internal promoter of choice.

Suitable SIN-LTR sequences will be well known to those of skill in the art (see e.g. Zufferey, R., et al., 1998. Journal of virology, 72(12), pp. 9873-9880 and Miyoshi, H., et al., 1998. Journal of virology, 72(10), pp. 8150-8157).

In some embodiments, the 5′ LTR comprises or consists of a nucleotide sequence which is at least 70% identical to SEQ ID NO: 24 or a fragment thereof. Suitably, the 5′ LTR comprises or consists of a nucleotide sequence which is at least 75%, at least 80%, at least 85%, at least 90%, at least 95%, at least 96%, at least 97%, at least 98%, or at least 99% identical to SEQ ID NO: 24 or a fragment thereof.

In some embodiments, the 5′ LTR comprises or consists of the nucleotide sequence SEQ ID NO: 24 or a fragment thereof.

Exemplary 5′ LTR
(SEQ ID NO: 24)
GGGTCTCTCTGGTTAGACCAGATCTGAGCCTGGGAGCTCTCTGGCTAACT
AGGGAACCCACTGCTTAAGCCTCAATAAAGCTTGCCTTGAGTGCTTCAAG
TAGTGTGTGCCCGTCTGTTGTGTGACTCTGGTAACTAGAGATCCCTCAGA
CCCTTTTAGTCAGTGTGGAAAATCTCTAGCAG

In some embodiments, the 5′ LTR and/or the 3′ LTR comprises or consists of a nucleotide sequence which is at least 70% identical to SEQ ID NO: 25 or a fragment thereof. Suitably, the 5′ LTR and/or the 3′ LTR comprises or consists of a nucleotide sequence which is at least 75%, at least 80%, at least 85%, at least 90%, at least 95%, at least 96%, at least 97%, at least 98%, or at least 99% identical to SEQ ID NO: 25 or a fragment thereof.

In some embodiments, the 5′ LTR and/or the 3′ LTR comprises or consists of the nucleotide sequence SEQ ID NO: 25 or a fragment thereof.

In some embodiments, the 5′ LTR and the 3′ LTR comprise or consist of a nucleotide sequence which is at least 70% identical to SEQ ID NO: 25 or a fragment thereof. Suitably, the 5′ LTR and the 3′ LTR comprise or consist of a nucleotide sequence which is at least 75%, at least 80%, at least 85%, at least 90%, at least 95%, at least 96%, at least 97%, at least 98%, or at least 99% identical to SEQ ID NO: 25 or a fragment thereof.

In some embodiments, the 5′ LTR and the 3′ LTR comprise or consist of the nucleotide sequence SEQ ID NO: 25 or a fragment thereof.

Exemplary 3′ LTR
(SEQ ID NO: 25)
TGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTC
AAGTAGTGTGTGCCCGTCTGTTGTGTGACTCTGGTAACTAGAGATCCCTC
AGACCCTTTTAGTCAGTGTGGAAAATCTCTAGCAG

### Primer Binding Site (PBS)

The lentiviral vector of the present invention may comprise a primer binding site (PBS). A PBS is a cis-acting element where a primer may bind to initiate reverse transcription of the RNA genome (see e.g. Lanchy, J. M., et al., 1998. Journal of Biological Chemistry, 273(38), pp. 24425-24432).

Suitable retroviral PBSs will be well known to those of skill in the art.

In some embodiments, a PBS comprises or consists of a nucleotide sequence which is at least 70% identical to SEQ ID NO: 26 or a fragment thereof. Suitably, a PBS comprises or consists of a nucleotide sequence which is at least 75%, at least 80%, at least 85%, at least 90%, at least 95%, at least 96%, at least 97%, at least 98%, or at least 99% identical to SEQ ID NO: 26 or a fragment thereof.

In some embodiments, a PBS comprises or consists of the nucleotide sequence SEQ ID NO: 26 or a fragment thereof.

Exemplary primer binding site
(SEQ ID NO: 26)
TGGCGCCCGAACAGGGACTTGAAAGCGAAAGGGAAACCAGAGGAGCTCTC
TCGACGCAGGACTCGGCTTGCTGAAGCGCGCACGGCAAGAGGCGAGGGGC
GGCGACTGGTGAGTACGCCAAAAATTTTGACTAGCGGAGGCTAGAAGGAG
AGAG

In some embodiments, a PBS comprises or consists of a nucleotide sequence which is at least 70% identical to SEQ ID NO: 27 or a fragment thereof. Suitably, a PBS comprises or consists of a nucleotide sequence which is at least 75%, at least 80%, at least 85%, at least 90%, at least 95%, at least 96%, at least 97%, at least 98%, or at least 99% identical to SEQ ID NO: 27 or a fragment thereof.

In some embodiments, a PBS comprises or consists of the nucleotide sequence SEQ ID NO: 27 or a fragment thereof.

Exemplary primer binding site
(SEQ ID NO: 27)
TGGCGCCCGAACAGGGACCTGAAAGCGAAAGGGAAACCAGAGCTCTCTCG
ACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGA
G

### Retroviral Psi Packaging Element

The lentiviral vector of the present invention may comprise a retroviral psi packaging element. As described above, a retroviral psi packaging element is a cis-acting element which is involved in regulating the essential process of packaging the retroviral RNA genome into the viral capsid during replication (see e.g. McBride, M. S., et al., 1997. Journal of virology, 71(6), pp. 4544-4554). A retroviral psi packaging element may form part of the 5′ region of the gag gene.

Suitable retroviral psi packaging elements will be well known to those of skill in the art.

In some embodiments, a retroviral psi packaging element comprises or consists of a nucleotide sequence which is at least 70% identical to SEQ ID NO: 28 or a fragment thereof. Suitably, a retroviral psi packaging element comprises or consists of a nucleotide sequence which is at least 75%, at least 80%, at least 85%, at least 90%, at least 95%, at least 96%, at least 97%, at least 98%, or at least 99% identical to SEQ ID NO: 28 or a fragment thereof.

In some embodiments, a retroviral psi packaging element comprises or consists of the nucleotide sequence SEQ ID NO: 28 or a fragment thereof.

Exemplary retroviral psi packaging element
(SEQ ID NO: 28)
ATGGGTGCGAGAGCGTCAGTATTAAGCGGGGGAGAATTAGATCGCGATGG
GAAAAAATTCGGTTAAGGCCAGGGGGAAAGAAAAAATATAAATTAAAACA
TATAGTATGGGCAAGCAGGGAGCTAGAACGATTCGCAGTTAATCCTGGCC
TGTTAGAAACATCAGAAGGCTGTAGACAAATACTGGGACAGCTACAACCA
TCCCTTCAGACAGGATCAGAAGAACTTAGATCATTATATAATACAGTAGC
AACCCTCTATTGTGTGCATCAAAGGATAGAGATAAAAGACACCAAGGAAG
CTTTAGACAAGATAGAGGAAGAGCAAAACAAAAGTAAGACCACCGCACAG
CTGATCAAGCGGCCG

### Rev Response Element (RRE)

The lentiviral vector of the present invention may comprise a rev response element (RRE). As described above, a RRE is a cis-acting element that enables the efficient export of RNA transcripts of the integrated provirus from the nucleus to the cytoplasm of an infected target cell (see e.g. Pollard, V. W. and Malim, M. H., 1998. Annual review of microbiology, 52(1), pp. 491-532).

Suitable RRE sequences will be well known to those of skill in the art.

In some embodiments, a RRE comprises or consists of a nucleotide sequence which is at least 70% identical to SEQ ID NO: 29 or a fragment thereof. Suitably, a RRE comprises or consists of a nucleotide sequence which is at least 75%, at least 80%, at least 85%, at least 90%, at least 95%, at least 96%, at least 97%, at least 98%, or at least 99% identical to SEQ ID NO: 29 or a fragment thereof.

In some embodiments, a RRE comprises or consists of the nucleotide sequence SEQ ID NO: 29 or a fragment thereof.

Exemplary rev response element
(SEQ ID NO: 29)
GGAGCTTTGTTCCTTGGGTTCTTGGGAGCAGCAGGAAGCACTATGGGCGC
AGCGTCAATGACGCTGACGGTACAGGCCAGACAATTATTGTCTGGTATAG
TGCAGCAGCAGAACAATTTGCTGAGGGCTATTGAGGCGCAACAGCATCTG
TTGCAACTCACAGTCTGGGGCATCAAGCAGCTCCAGGCAAGAATCCTGGC
TGTGGAAAGATACCTAAAGGATCAACAGCTCCTGGGGATTT

In some embodiments, a RRE comprises or consists of a nucleotide sequence which is at least 70% identical to SEQ ID NO: 30 or a fragment thereof. Suitably, a RRE comprises or consists of a nucleotide sequence which is at least 75%, at least 80%, at least 85%, at least 90%, at least 95%, at least 96%, at least 97%, at least 98%, or at least 99% identical to SEQ ID NO: 30 or a fragment thereof.

In some embodiments, a RRE comprises or consists of the nucleotide sequence SEQ ID NO: 30 or a fragment thereof.

Exemplary rev response element
(SEQ ID NO: 30)
GGAGCTTTGTTCCTTGGGTTCTTGGGAGCAGCAGGAAGCACTATGGGCGC
AGCCTCAATGACGCTGACGGTACAGGCCAGACAATTATTGTCTGGTATAG
TGCAGCAGCAGAACAATTTGCTGAGGGCTATTGAGGCGCAACAGCATCTG
TTGCAACTCACAGTCTGGGGCATCAAGCAGCTCCAGGCAAGAATCCTGGC
TGTGGAAAGATACCTAAAGGATCAACAGCTCCTGGGGATTT

Central Polypurine Tract (cPPT)

The lentiviral vector of the present invention may comprise a central polypurine tract (cPPT). As described above, a cPPT may allow initiation of plus-strand synthesis (see e.g. Follenzi, A. et al., 2000. Nature genetics, 25(2), pp. 217-222).

Suitable cPPT sequences will be well known to those of skill in the art.

In some embodiments, a cPPT comprises or consists of a nucleotide sequence which is at least 70% identical to SEQ ID NO: 31 or a fragment thereof. Suitably, a cPPT comprises or consists of a nucleotide sequence which is at least 75%, at least 80%, at least 85%, at least 90%, at least 95%, at least 96%, at least 97%, at least 98%, or at least 99% identical to SEQ ID NO: 31 or a fragment thereof.

In some embodiments, a cPPT comprises or consists of the nucleotide sequence SEQ ID NO: 31 or a fragment thereof.

Exemplary central polypurine tract
(SEQ ID NO: 31)
AACTTTTAAAAGAAAAGGGGGGATTGGGGGGTACAGTGCAGGGGAAAGAA
TAGTAGACATAATAGCAACAGACATACAAACTAAAGAATTACAAAAACAA
ATTACAAAAATTCAAAATTTTATC

### Other Elements

The lentiviral vector of the present invention may comprise any other suitable elements.

In some embodiments, the lentiviral vector of the present invention comprises an element comprising or consisting of a nucleotide sequence which is at least 70%, at least 75%, at least 80%, at least 85%, at least 90%, at least 95%, at least 96%, at least 97%, at least 98%, or at least 99% identical to SEQ ID NO: 32 or a fragment thereof. In some embodiments, the lentiviral vector of the present invention comprises an element comprising or consisting of the nucleotide sequence of SEQ ID NO: 32 or a fragment thereof.

Exemplary delta ENV1
(SEQ ID NO: 32)
TCTTCAGACCTGGAGGAGGAGATATGAGGGACAATTGGAGAAGTGAATTA
TATAAATATAAAGTAGTAAAAATTGAACCATTAGGAGTAGCACCCACCAA
GGCAAAGAGAAGAGTGGTGCAGAGAGAAAAAAGAGCAGTGGGAATA

In some embodiments, the lentiviral vector of the present invention comprises an element comprising or consisting of a nucleotide sequence which is at least 70%, at least 75%, at least 80%, at least 85%, at least 90%, at least 95%, at least 96%, at least 97%, at least 98%, or at least 99% identical to SEQ ID NO: 33 or a fragment thereof. In some embodiments, the lentiviral vector of the present invention comprises an element comprising or consisting of the nucleotide sequence of SEQ ID NO: 33 or a fragment thereof.

Exemplary delta ENV2
(SEQ ID NO: 33)
GGGTTGCTCTGGAAAACTCATTTGCACCACTGCTGTGCCTTGGAATGCTA
GTTGGAGTAATAAATCTCTGGAACAGATTTGGAATCACACGACCTGGATG
GAGTGGGACAGAGAAATTAACAATTACACAAGCTTAATACACTCCTTAAT
TGAAGAATCGCAAAACCAGCAAGAAAAGAATGAACAAGAATTATTGGAAT
TAGATAAATGGGCAAGTTTGTGGAATTGGTTTAACATAACAAATTGGCTG
TGGTATATAAAATTATTCATAATGATAGTAGGAGGCTTGGTAGGTTTAAG
AATAGTTTTTGCTGTACTTTCTATAGTGAATAGAGTTAGGCAGGGATATT
CACCATTATCGTTTCAGACCCACCTCCCAACCCCGAGGGGACCCGACAGG
CCCGAAGGAATAGAAGAAGAAGGTGGAGAGAGAGACAGAGACAGATCCAT
TCGATTAGTGAACGGATC

### Exemplary Cis-Acting Elements

The lentiviral vector of the present invention may comprise a cis-acting element comprising a PBS, a retroviral psi packaging element, and a rev response element (RRE).

In some embodiments, a cis-acting element comprising a PBS, a retroviral psi packaging element, and a RRE comprises or consists of a nucleotide sequence which is at least 70% identical to SEQ ID NO: 34 or a fragment thereof. Suitably, a cis-acting element comprising a PBS, a retroviral psi packaging element, and a RRE comprises or consists of a nucleotide sequence which is at least 75%, at least 80%, at least 85%, at least 90%, at least 95%, at least 96%, at least 97%, at least 98%, or at least 99% identical to SEQ ID NO: 34 or a fragment thereof.

In some embodiments, a cis-acting element comprising a PBS, a retroviral psi packaging element, and a RRE comprises or consists of the nucleotide sequence SEQ ID NO: 34 or a fragment thereof.

Exemplary cis-acting element comprising a
PBS, a retroviral psi packaging element,
and a RRE
(SEQ ID NO: 34)
TGGCGCCCGAACAGGGACCTGAAAGCGAAAGGGAAACCAGAGCTCTCTCG
ACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGA
GATGGGTGCGAGAGCGTCAGTATTAAGCGGGGGAGAATTAGATCGCGATG
GGAAAAAATTCGGTTAAGGCCAGGGGGAAAGAAAAAATATAAATTAAAAC
ATATAGTATGGGCAAGCAGGGAGCTAGAACGATTCGCAGTTAATCCTGGC
CTGTTAGAAACATCAGAAGGCTGTAGACAAATACTGGGACAGCTACAACC
ATCCCTTCAGACAGGATCAGAAGAACTTAGATCATTATATAATACAGTAG
CAACCCTCTATTGTGTGCATCAAAGGATAGAGATAAAAGACACCAAGGAA
GCTTTAGACAAGATAGAGGAAGAGCAAAACAAAAGTAAGACCACCGCACA
GCAAGCGGCCGCTGATCTTCAGACCTGGAGGAGGAGATATGAGGGACAAT
TGGAGAAGTGAATTATATAAATATAAAGTAGTAAAAATTGAACCATTAGG
AGTAGCACCCACCAAGGCAAAGAGAAGAGTGGTGCAGAGAGAAAAAAGAG
CAGTGGGAATAGGAGCTTTGTTCCTTGGGTTCTTGGGAGCAGCAGGAAGC
ACTATGGGCGCAGCCTCAATGACGCTGACGGTACAGGCCAGACAATTATT
GTCTGGTATAGTGCAGCAGCAGAACAATTTGCTGAGGGCTATTGAGGCGC
AACAGCATCTGTTGCAACTCACAGTCTGGGGCATCAAGCAGCTCCAGGCA
AGAATCCTGGCTGTGGAAAGATACCTAAAGGATCAACAGCTCCTGGGGAT
TT

The lentiviral vector of the present invention may comprise a cis-acting element comprising a PBS, a retroviral psi packaging element, a rev response element (RRE), and a central polypurine tract (cPPT).

In some embodiments, a cis-acting element comprising a PBS, a retroviral psi packaging element, a RRE, and a cPPT comprises or consists of a nucleotide sequence which is at least 70% identical to SEQ ID NO: 35 or a fragment thereof. Suitably, a cis-acting element comprising a PBS, a retroviral psi packaging element, a RRE, and a cPPT comprises or consists of a nucleotide sequence which is at least 75%, at least 80%, at least 85%, at least 90%, at least 95%, at least 96%, at least 97%, at least 98%, or at least 99% identical to SEQ ID NO: 35 or a fragment thereof.

In some embodiments, a cis-acting element comprising a PBS, a retroviral psi packaging element, a RRE, and a cPPT comprises or consists of the nucleotide sequence SEQ ID NO: 35 or a fragment thereof.

Exemplary cis-acting element comprising a
PBS, a retroviral psi packaging element,
a RRE, and a CPPT
(SEQ ID NO: 35)
TGGCGCCCGAACAGGGACCTGAAAGCGAAAGGGAAACCAGAGCTCTCTCG
ACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGA
GATGGGTGCGAGAGCGTCAGTATTAAGCGGGGGAGAATTAGATCGCGATG
GGAAAAAATTCGGTTAAGGCCAGGGGGAAAGAAAAAATATAAATTAAAAC
ATATAGTATGGGCAAGCAGGGAGCTAGAACGATTCGCAGTTAATCCTGGC
CTGTTAGAAACATCAGAAGGCTGTAGACAAATACTGGGACAGCTACAACC
ATCCCTTCAGACAGGATCAGAAGAACTTAGATCATTATATAATACAGTAG
CAACCCTCTATTGTGTGCATCAAAGGATAGAGATAAAAGACACCAAGGAA
GCTTTAGACAAGATAGAGGAAGAGCAAAACAAAAGTAAGACCACCGCACA
GCAAGCGGCCGCTGATCTTCAGACCTGGAGGAGGAGATATGAGGGACAAT
TGGAGAAGTGAATTATATAAATATAAAGTAGTAAAAATTGAACCATTAGG
AGTAGCACCCACCAAGGCAAAGAGAAGAGTGGTGCAGAGAGAAAAAAGAG
CAGTGGGAATAGGAGCTTTGTTCCTTGGGTTCTTGGGAGCAGCAGGAAGC
ACTATGGGCGCAGCCTCAATGACGCTGACGGTACAGGCCAGACAATTATT
GTCTGGTATAGTGCAGCAGCAGAACAATTTGCTGAGGGCTATTGAGGCGC
AACAGCATCTGTTGCAACTCACAGTCTGGGGCATCAAGCAGCTCCAGGCA
AGAATCCTGGCTGTGGAAAGATACCTAAAGGATCAACAGCTCCTGGGGAT
TTGGGGTTGCTCTGGAAAACTCATTTGCACCACTGCTGTGCCTTGGAATG
CTAGTTGGAGTAATAAATCTCTGGAACAGATTTGGAATCACACGACCTGG
ATGGAGTGGGACAGAGAAATTAACAATTACACAAGCTTAATACACTCCTT
AATTGAAGAATCGCAAAACCAGCAAGAAAAGAATGAACAAGAATTATTGG
AATTAGATAAATGGGCAAGTTTGTGGAATTGGTTTAACATAACAAATTGG
CTGTGGTATATAAAATTATTCATAATGATAGTAGGAGGCTTGGTAGGTTT
AAGAATAGTTTTTGCTGTACTTTCTATAGTGAATAGAGTTAGGCAGGGAT
ATTCACCATTATCGTTTCAGACCCACCTCCCAACCCCGAGGGGACCCGAC
AGGCCCGAAGGAATAGAAGAAGAAGGTGGAGAGAGAGACAGAGACAGATC
CATTCGATTAGTGAACGGATCTCGACGGTATCGGTTAACTTTTAAAAGAA
AAGGGGGGATTGGGGGGTACAGTGCAGGGGAAAGAATAGTAGACATAATA
GCAACAGACATACAAACTAAAGAATTACAAAAACAAATTACAAAAATTCA
AAATTTTATC

### Exemplary Lentiviral Genomes

The lentiviral genome of the present invention may comprise from 5′ to 3′: a 5′ LTR, one or more cis-acting elements, a protein-coding sequence, optionally one or more post-transcriptional regulatory sequences, and a 3′ LTR.

For example, the lentiviral genome of the present invention may comprise from 5′ to 3′: a 5′ LTR, a PBS, a retroviral psi packaging element, a RRE, a cPPT, a liver-specific promoter, a protein-coding sequence, a WPRE, one or more miRNA target sequence, and a 3′ LTR.

The lentiviral genome of the present invention may further comprise any other suitable elements, such as any other elements described herein or one or more spacer sequence. The spacer sequence(s) may comprise, for example, at least one (e.g. 1, 2, 3, 4, 5, 6, 7, 8, 9, 10), at least two, at least three, at least four, at least five, at least six, at least seven, at least eight, at least nine, or at least ten nucleotide bases.

In some embodiments, the lentiviral genome comprises or consists of a nucleotide sequence which is at least 70% identical to SEQ ID NO: 36 or a fragment thereof. Suitably, the lentiviral genome comprises or consists of a nucleotide sequence which is at least 75%, at least 80%, at least 85%, at least 90%, at least 95%, at least 96%, at least 97%, at least 98%, or at least 99% identical to SEQ ID NO: 36 or a fragment thereof.

In some embodiments, the lentiviral genome comprises or consists of the nucleotide sequence SEQ ID NO: 36 or a fragment thereof.

Exemplary lentiviral genome
(SEQ ID NO: 36)
GGGTCTCTCTGGTTAGACCAGATCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTGTGACTCTGGTAACTAGAGATCC
CTCAGACCCTTTTAGTCAGTGTGGAAAATCTCTAGCAGTGGCGCCCGAACAGGGACCTGAAAGCGAAAGGGA
AACCAGAGCTCTCTCGACGCAGGACTCGGCTTGCTGAAGCGCGCACGGCAAGAGGCGAGGGGCGGCGACTGG
TGAGTACGCCAAAAATTTTGACTAGCGGAGGCTAGAAGGAGAGAGATGGGTGCGAGAGCGTCAGTATTAAGC
GGGGGAGAATTAGATCGCGATGGGAAAAAATTCGGTTAAGGCCAGGGGGAAAGAAAAAATATAAATTAAAAC
ATATAGTATGGGCAAGCAGGGAGCTAGAACGATTCGCAGTTAATCCTGGCCTGTTAGAAACATCAGAAGGCT
GTAGACAAATACTGGGACAGCTACAACCATCCCTTCAGACAGGATCAGAAGAACTTAGATCATTATATAATA
CAGTAGCAACCCTCTATTGTGTGCATCAAAGGATAGAGATAAAAGACACCAAGGAAGCTTTAGACAAGATAG
AGGAAGAGCAAAACAAAAGTAAGACCACCGCACAGCAAGCGGCCGCTGATCTTCAGACCTGGAGGAGGAGAT
ATGAGGGACAATTGGAGAAGTGAATTATATAAATATAAAGTAGTAAAAATTGAACCATTAGGAGTAGCACCC
ACCAAGGCAAAGAGAAGAGTGGTGCAGAGAGAAAAAAGAGCAGTGGGAATAGGAGCTTTGTTCCTTGGGTTC
TTGGGAGCAGCAGGAAGCACTATGGGCGCAGCCTCAATGACGCTGACGGTACAGGCCAGACAATTATTGTCT
GGTATAGTGCAGCAGCAGAACAATTTGCTGAGGGCTATTGAGGCGCAACAGCATCTGTTGCAACTCACAGTC
TGGGGCATCAAGCAGCTCCAGGCAAGAATCCTGGCTGTGGAAAGATACCTAAAGGATCAACAGCTCCTGGGG
ATTTGGGGTTGCTCTGGAAAACTCATTTGCACCACTGCTGTGCCTTGGAATGCTAGTTGGAGTAATAAATCT
CTGGAACAGATTTGGAATCACACGACCTGGATGGAGTGGGACAGAGAAATTAACAATTACACAAGCTTAATA
CACTCCTTAATTGAAGAATCGCAAAACCAGCAAGAAAAGAATGAACAAGAATTATTGGAATTAGATAAATGG
GCAAGTTTGTGGAATTGGTTTAACATAACAAATTGGCTGTGGTATATAAAATTATTCATAATGATAGTAGGA
GGCTTGGTAGGTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGAAGAAGGTGGA
GAGAGAGACAGAGACAGATCCATTCGATTAGTGAACGGATCTCGACGGTATCGGTTAACTTTTAAAAGAAAA
GGGGGGATTGGGGGGTACAGTGCAGGGGAAAGAATAGTAGACATAATAGCAACAGACATACAAACTAAAGAA
TTACAAAAACAAATTACAAAAATTCAAAATTTTATCGATCACGAGACTAGCCTCGAGCACGCGAGTTAATAA
TTACCAGCGCGGGCCAAATAAATAATCCGCGAGGGGCAGGTGACGTTTGCCCAGCGCGCGCTGGTAATTATT
AACCTCGCGAATATTGATTCGAGGCCGCGATTGCCGCAATCGCGAGGGGCAGGTGACCTTTGCCCAGCGCGC
GTTCGCCCCGCCCCGGACGGTATCGATAAGCTTAGGAGCTTGGGCTGCAGGTCGAGGGCACTGGGAGGATGT
TGAGTAAGATGGAAAACTACTGATGACCCTTGCAGAGACAGAGTATTAGGACATGTTTGAACAGGGGCCGGG
CGATCAGCAGGTAGCTCTAGAGGATCCCCGTCTGTCTGCACATTTCGTAGAGCGAGTGTTCCGATACTCTAA
TCTCCCTAGGCAAGGTTCATATTTGTGTAGGTTACTTATTCTCCTTTTGTTGACTAAGTCAATAATCAGAAT
CAGCAGGTTTGGAGTCAGCTTGGCAGGGATCAGCAGCCTGGGTTGGAAGGAGGGGGTATAAAAGCCCCTTCA
CCAGGAGAAGCCGTCACACAGATCCACAAGCTCCTGGCTAGCGTACGCCACCATGCTGAGGGCCAAGAACCA
GCTGTTCCTGCTGTCCCCACACTACCTGCGCCAGGTGAAGGAGAGCTCCGGCTCTAGGCTGATCCAGCAGCG
CCTGCTGCACCAGCAGCAGCCACTGCACCCTGAGTGGGCCGCCCTGGCCAAGAAGCAGCTGAAGGGCAAGAA
TCCCGAGGACCTGATCTGGCACACCCCAGAGGGCATCAGCATCAAGCCCCTGTATTCCAAGAGGGACACCAT
GGATCTGCCAGAGGAGCTGCCAGGAGTGAAGCCTTTCACACGCGGCCCTTACCCAACCATGTATACATTTCG
GCCCTGGACCATCAGACAGTACGCCGGCTTCAGCACAGTGGAGGAGTCCAACAAGTTTTATAAGGACAATAT
CAAGGCAGGACAGCAGGGCCTGAGCGTGGCCTTTGATCTGGCCACACACCGCGGCTACGACTCCGATAACCC
CAGGGTGCGCGGCGACGTGGGAATGGCAGGAGTGGCAATCGACACCGTGGAGGATACAAAGATCCTGTTCGA
TGGCATCCCTCTGGAGAAGATGTCCGTGTCTATGACCATGAACGGCGCCGTGATCCCCGTGCTGGCCAACTT
CATCGTGACAGGCGAGGAGCAGGGCGTGCCTAAGGAGAAGCTGACCGGCACAATCCAGAACGACATCCTGAA
GGAGTTCATGGTGCGGAATACCTATATCTTTCCCCCTGAGCCATCCATGAAGATCATCGCCGACATCTTCGA
GTACACAGCCAAGCACATGCCCAAGTTTAATAGCATCTCCATCTCTGGCTATCACATGCAGGAGGCAGGAGC
AGACGCAATCCTGGAGCTGGCCTACACCCTGGCCGATGGCCTGGAGTATTCCCGGACCGGCCTGCAGGCAGG
CCTGACAATCGACGAGTTCGCCCCTAGACTGTCTTTCTTTTGGGGCATCGGCATGAACTTTTACATGGAGAT
CGCCAAGATGAGGGCAGGCCGGAGACTGTGGGCTCACCTGATCGAGAAGATGTTCCAGCCTAAGAATAGCAA
GTCCCTGCTGCTGAGAGCCCACTGCCAGACCTCCGGATGGAGCCTGACAGAGCAGGACCCATATAACAATAT
CGTGAGAACCGCCATCGAGGCAATGGCAGCCGTGTTCGGAGGAACCCAGTCTCTGCACACAAACAGCTTTGA
TGAGGCCCTGGGCCTGCCTACCGTGAAGAGCGCCCGGATCGCCAGAAATACACAGATCATCATCCAGGAGGA
GTCTGGCATCCCAAAGGTGGCAGATCCATGGGGAGGAAGCTACATGATGGAGTGCCTGACAAACGACGTGTA
TGATGCCGCCCTGAAGCTGATCAATGAGATCGAGGAGATGGGAGGAATGGCAAAGGCAGTGGCAGAGGGAAT
CCCAAAGCTGCGGATCGAGGAGTGTGCAGCAAGGCGCCAGGCAAGAATCGACTCTGGCAGCGAAGTGATCGT
GGGCGTGAACAAGTACCAGCTGGAGAAGGAGGACGCCGTGGAGGTGCTGGCCATCGATAACACCTCCGTGCG
GAATAGACAGATCGAGAAGCTGAAGAAGATCAAGTCTAGCCGGGATCAGGCCCTGGCCGAGAGATGCCTGGC
CGCCCTGACAGAGTGTGCAGCCTCTGGCGACGGAAACATCCTGGCCCTGGCCGTGGATGCCAGCCGGGCCAG
ATGTACCGTGGGAGAGATCACAGACGCCCTGAAGAAGGTGTTCGGCGAGCACAAGGCCAATGATAGGATGGT
GAGCGGCGCCTATCGCCAGGAGTTCGGCGAGTCCAAGGAGATCACCTCTGCCATCAAGAGGGTGCACAAGTT
TATGGAGAGGGAGGGCAGACGGCCCAGGCTGCTGGTGGCAAAGATGGGACAGGACGGACACGATAGGGGAGC
CAAAGTGATCGCAACCGGCTTCGCAGACCTGGGATTTGACGTGGACATCGGCCCTCTGTTCCAGACACCACG
GGAGGTGGCACAGCAGGCAGTGGACGCAGATGTGCACGCAGTGGGCATCAGCACCCTGGCCGCAGGACACAA
GACACTGGTGCCTGAGCTGATCAAGGAGCTGAACTCCCTGGGCAGACCAGACATCCTGGTCATGTGCGGCGG
CGTGATCCCACCCCAGGATTACGAGTTCCTGTTTGAAGTGGGCGTGTCTAACGTGTTCGGCCCTGGAACCAG
GATCCCTAAGGCAGCCGTGCAGGTGCTGGACGACATCGAGAAGTGTCTGGAGAAGAAGCAGCAGAGCGTGTG
ATGAGTCGACTCGACAATCAACCTCTGGATTACAAAATTTGTGAAAGATTGACTGGTATTCTTAACTATGTT
GCTCCTTTTACGCTATGTGGATACGCTGCTTTAATGCCTTTGTATCATGCTATTGCTTCCCGTATGGCTTTC
ATTTTCTCCTCCTTGTATAAATCCTGGTTGCTGTCTCTTTATGAGGAGTTGTGGCCCGTTGTCAGGCAACGT
GGCGTGGTGTGCACTGTGTTTGCTGACGCAACCCCCACTGGTTGGGGCATTGCCACCACCTGTCAGCTCCTT
TCCGGGACTTTCGCTTTCCCCCTCCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGAAATCATCGTCCTTTCCTTGGCTG
CTCGCCTGTGTTGCCACCTGGATTCTGCGCGGGACGTCCTTCTGCTACGTCCCTTCGGCCCTCAATCCAGCG
GACCTTCCTTCCCGCGGCCTGCTGCCGGCTCTAGATAATCCATAAAGTAGGAAACACTACACGATTCCATAA
AGTAGGAAACACTACAACCGGTTCCATAAAGTAGGAAACACTACATCACTCCATAAAGTAGGAAACACTACA
CCCGGGTCGAGCTCGGTACCTTTAAGACCAATGACTTACAAGGCAGCTGTAGATCTTAGCCACTTTTTAAAA
GAAAAGGGGGGACTGGAAGGGCTAATTCACTCCCAACGAAGACAAGATCTGCTTTTTGCTTGTACTGGGTCT
CTCTGGTTAGACCAGATCTGAGCCTGGGAGCTCTCTGGCTAACTAGGGAACCCACTGCTTAAGCCTCAATAA
AGCTTGCCTTGAGTGCTTCAAGTAGTGTGTGCCCGTCTGTTGTGTGACTCTGGTAACTAGAGATCCCTCAGA
CCCTTTTAGTCAGTGTGGAAAATCTCTAGCA

### Variants, Derivatives, Analogues, and Fragments

In addition to the specific polypeptides and polynucleotides mentioned herein, the invention also encompasses variants, derivatives, and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one or all of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one or all of its endogenous functions.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Polypeptides used in the invention may also have deletions, insertions or substitutions of amino acid residues which produce a silent change and result in a functionally equivalent polypeptide. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The effect of additions, deletions, substitutions, modifications, replacements and/or variations may be predicted using any suitable prediction tool e.g. SIFT (Vaser, R., et al., 2016. Nature protocols, 11(1), pp. 1-9), PolyPhen-2 (Adzhubei, I., et al., 2013. Current protocols in human genetics, 76(1), pp. 7-20), CADD (Rentzsch, P., et al., 2021. Genome medicine, 13(1), pp. 1-12), REVEL (Ioannidis, N.M., et al., 2016. The American Journal of Human Genetics, 99(4), pp. 877-885), MetaLR (Dong, C., et al., 2015. Human molecular genetics, 24(8), pp. 2125-2137), and/or MutationAssessor (Reva, B., et al., 2011. Nucleic acids research, 39(17), pp.e118-e118) or based on clinical data e.g. ClinVar (Landrum, M. J., et al., 2016. Nucleic acids research, 44(D1), pp.D862-D868). Suitable additions, deletions, substitutions, modifications, replacements and/or variations may be considered tolerated, benign, and/or likely benign.

Typically, a variant may have a certain identity with the wild type amino acid sequence or the wild type nucleotide sequence.

In the present context, a variant sequence is taken to include an amino acid sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs detailed herein refers to a sequence which has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (see e.g. Devereux, J., et al., 1984. Nucleic acids research, 12(1), pp. 387-395). Examples of other software that can perform sequence comparisons include, but are not limited to, the BLAST package (see e.g. Altschul, S. F., et al., 1990. Journal of molecular biology, 215(3), pp. 403-410), BLAST 2 (see e.g. Tatusova, T. A. and Madden, T. L., 1999. FEMS microbiology letters, 174(2), pp. 247-250), FASTA (see e.g. Pearson, W. R. and Lipman, D. J., 1988. PNAS, 85(8), pp. 2444-2448.), EMBOSS Needle (Madeira, F., et al., 2019. Nucleic acids research, 47(W1), pp.W636-W641) and the GENEWORKS suite of comparison tools. For some applications, it is preferred to use EMBOSS Needle.

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives, and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded polypeptide. These methods are only illustrative of the numerous standard techniques known in the art for manipulation of DNA sequences and other known techniques may also be used.

### Method of Production

In one aspect, the present invention provides a method of producing a lentiviral vector according to the present invention.

Suitable methods to produce lentiviral vectors will be well known to those of skill in the art (see e.g. Merten, O. W., et al., 2016. Molecular Therapy-Methods & Clinical Development, 3, p.16017).

The method of production may comprise: (a) introducing a transfer vector and one or more helper vectors into a host cell; (b) culturing the host cell under conditions suitable to produce lentiviral vectors according to the present invention; and (c) obtaining the lentiviral vectors from the host cell.

As used herein, a “transfer vector” may encode the lentiviral genome of the present invention. Suitably, the transfer vector used to produce the lentiviral viral genome within a host cell/packaging cell will have sufficient lentiviral genetic information to allow packaging of an RNA genome, in the presence of packaging components (e.g. gag-pol, rev, env), into a viral particle which is capable of infecting a target cell, but is incapable of independent replication to produce infectious viral particles within the final target cell.

The transfer vector used to produce the viral genome within a host cell/packaging cell may include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter). The transfer vector may be a plasmid.

As used herein, a “helper vector” may encode one or more packaging components (e.g. gag-pol, rev, env). The nucleotide sequence encoding the packaging component(s) may be operably linked to a promoter (e.g. a CMV promoter or a RSV promoter) and/or a polyadenylation signal. The term “helper vector” may include “packaging vectors” (e.g. encoding gag-pol or rev) and “envelope vectors” (e.g. encoding an env gene, such as VSV-g). The helper vectors, packaging vectors, and/or envelope vectors may be plasmids.

The transfer vector and one or more helper vectors may be introduced into the host cell by any suitable technique known in the art, such as transfection, transduction and/or transformation. Suitably, the helper vectors may be transiently transfected or transduced into the host cell or may be stably maintained (e.g. stably integrated into the cell genome or episomally maintained) within the host cell. Alternatively, a combination of transient transfection or transduction and stable maintenance may be used to introduce the helper vectors into the host cell.

Suitably, the transfer vector and/or the helper vectors may be plasmids and introduced by transfection. Suitably, a four plasmid system may be used consisting of a transfer plasmid and three helper plasmids. The three helper plasmids may consist of: a first helper plasmid encoding a gag-pol gene; a second helper plasmid encoding a rev gene; and a third helper plasmid encoding an env gene. Alternatively, a three plasmid system may be used which consists of a transfer plasmid, one helper plasmid encoding a gag-pol gene and a rev gene; and one helper plasmid encoding an env gene. Alternatively, a two plasmid system may be used in which all helper functions (e.g. gag-pol, rev, and env) are encoded by one helper plasmid.

Any suitable host cell may be used to produce the lentiviral vector. Suitable host cells include producer cells and packaging cells, such as those described below (e.g. HEK 293, or derivatives thereof). Suitable conditions for culturing the host cell will be well known to the skilled person. For example, the host cells may be incubated in chemically defined medium for from about 1 day to about 5 days (e.g. about 48 hours, about 72 hours, or about 96 hours).

The lentiviral vector may be obtained using in any suitable methods known in the art. For example the culture supernatant may be harvested and lentiviral vector subsequently purified from the culture supernatant (e.g. by centrifugation, membrane filtration and/or chromatography). The method of production may further comprise any other suitable process steps e.g. DNA reduction, concentration, formulation and/or sterilization.

### Vectors, Kits and Systems

In one aspect, the present invention provides a vector encoding the lentiviral genome of the present invention. The vector may be a transfer vector, as described herein. For example, the vector may be a plasmid and/or the lentiviral genome may be operably linked to a promoter (e.g. a viral promoter, such as a CMV promoter).

In one aspect, the present invention provides a kit or system for producing the lentiviral vector of the present invention.

The kit or system may be a lentivirus packaging kit or system or a lentivirus production kit or system. As used herein, a “lentivirus packaging kit or system” may comprise one or more components, and optionally instructions, for packaging the lentiviral vector of the present invention. As used herein, a “lentivirus production kit or system” may comprise one or more components, and optionally instructions, for producing the lentiviral vector of the present invention.

The kit or system may comprise a transfer vector encoding the lentivirus genome of the present invention and optionally one or more helper vectors. The kit or system may further comprise host cells (e.g. packaging cells or producer cells) and/or other reagents (e.g. transfection reagent, culture medium, etc.). The kit or system may further comprise any other suitable components, and optionally instructions for packaging and/or producing the lentiviral vector of the present invention.

### Cells

In one aspect, the present invention provides a cell comprising the lentiviral vector of the present invention. The cell may be an isolated cell. Suitably, the cell is a mammalian cell, for example a human cell. The cell may be an isolated human cell.

Suitably, the cell may be a producer cell. The term “producer cell” includes a cell that produces viral particles, after transient transfection, stable transfection or vector transduction of all the elements necessary to produce the viral particles or any cell engineered to stably comprise the elements necessary to produce the viral particles. Suitable producer cells will be well known to those of skill in the art and may include HEK293, COS-1, COS-7, CV-1, HeLa, CHO, and A549 cell lines. In some embodiments, the producer cell is a HEK293 cell, or a derivative thereof (e.g. a HEK293T cell, a HEK293T Lenti-X, a HEK293T-Rex cell, a HEK293FT cell, a HEK293SF-3F6 cell, a HEK293SF-3F9 cell, a HEK293-EBNA1 cell, or a SJ293TS cell).

Suitably, the cell may be a packaging cell. The term “packaging cell” includes a cell which contains some or all of the elements necessary for packaging a recombinant virus genome. Typically, such packaging cells contain one or more vectors which are capable of expressing viral structural proteins (e.g. gag-pol, rev, env) and/or one or more genes encoding the viral structural proteins have been integrated into the genome of the packaging cell. Cells comprising only some of the elements required for the production of enveloped viral particles are useful as intermediate reagents in the generation of viral particle producer cell lines, through subsequent steps of transient transfection, transduction or stable integration of each additional required element. These intermediate reagents are encompassed by the term “packaging cell”. Suitable packaging cells will be well known to those of skill in the art (see e.g. Merten, O. W., et al., 2016. Molecular Therapy-Methods & Clinical Development, 3, p.16017).

Suitably, the cell may be a liver cell, for example a hepatocyte. Suitably, the cell may be an immortalized liver cell, for example an immortalized hepatocyte. Suitable cell lines will be well known to those of skill in the art, for example HepG2, Hep3B, HBG, and HepaRG cell lines. Methods to generate immortalized liver cells (e.g. immortalized hepatocytes) will be well known to those of skill in the art (see e.g. Ramboer, E., et al., 2015. Methods Mol Biol, 1250, pp. 53-76). Suitably, the cell may be a stem cell.

### Pharmaceutical Compositions

In one aspect, the present invention provides pharmaceutical composition comprising the lentiviral vector or cell of the present invention. In preferred embodiments, the pharmaceutical composition comprises the lentiviral vector of the present invention in the form of a lentiviral particle.

A pharmaceutical composition is a composition that comprises or consists of a therapeutically effective amount of a pharmaceutically active agent e.g. the lentiviral vector. A pharmaceutical composition preferably includes a pharmaceutically acceptable carrier, diluent or excipient (including combinations thereof).

By “pharmaceutically acceptable” is included that the formulation is sterile and pyrogen free. The carrier, diluent, and/or excipient must be “acceptable” in the sense of being compatible with the lentiviral vector and not deleterious to the recipients thereof. Typically, the carriers, diluents, and excipients will be saline or infusion media which will be sterile and pyrogen free, however, other acceptable carriers, diluents, and excipients may be used.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as (or in addition to) the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The lentiviral vector, cell, or pharmaceutical composition according to the present invention may be administered in a manner appropriate for treating and/or preventing the diseases described herein. Suitable administration routes will be known to the skilled person (see e.g. Fumoto, S., et al., 2013. Novel Gene Therapy Approaches, pp. 3-31). The quantity and frequency of administration may be determined by the skilled person, for example depending by such factors as the condition of the subject, and the type and severity of the subject's disease. The pharmaceutical composition may be formulated accordingly.

The lentiviral vector, cell or pharmaceutical composition according to the present invention may be administered parenterally, (e.g. intravenous, intra-arterial, intramuscular, intrathecal, subcutaneous), or by infusion techniques. The lentiviral vector, cell or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The lentiviral vector, cell or pharmaceutical composition according to the present invention may be administered systemically, for example by intravenous injection or intraperitoneal injection. In some embodiments, the lentiviral vector, cell or pharmaceutical composition according to the present invention is administered by intravenous injection. The pharmaceutical composition may be formulated accordingly.

The lentiviral vector, cell or pharmaceutical composition according to the present invention may be administered locally, for example by direct injection, intra-arterial injection, or intraportal injection. In some embodiments, the lentiviral vector, cell or pharmaceutical composition according to the present invention is administered locally to the liver. In some embodiments, the lentiviral vector, cell or pharmaceutical composition according to the present invention is administered by intrahepatic injection, intrahepatic arterial injection, or intraportal injection. The pharmaceutical composition may be formulated accordingly.

The pharmaceutical compositions may comprise lentiviral vectors or cells of the invention in infusion media, for example sterile isotonic solution. The pharmaceutical composition may be enclosed in ampoules, disposable syringes or multiple dose vials made of glass or plastic.

The lentiviral vector, cell or pharmaceutical composition may be administered in a single or in multiple doses. Suitably, the lentiviral vector, cell or pharmaceutical composition may be administered in a single, one off dose. The pharmaceutical composition may be formulated accordingly.

The lentiviral vector, cell or pharmaceutical composition may be administered at varying doses (e.g. measured in Transducing Units (TU) per kg). The physician in any event may determine the actual dosage which will be most suitable for any individual subject and the dosage may, for example, vary with the age, weight and response of the particular subject. Suitably, the lentiviral vector of the present invention is administered at a dose of at least about 10⁸ TU/kg, at least about 10⁹ TU/kg, or at least about 10¹⁰ TU/kg. Suitably, the lentiviral vector of the present invention is administered at a dose of about 10¹³ TU/kg or less, about 10¹² TU/kg or less, or about 10¹¹ TU/kg or less. Suitably, the lentiviral vector of the present invention is administered in a dose of from about 10⁸ to about 10¹³ TU/kg, from about 10⁹ to about 10¹³ TU/kg, or from about 10¹⁰ to about 10¹³ TU/kg. Suitably, the lentiviral vector of the present invention is administered in a dose of from about 10⁸ to about 10¹² TU/kg, from about 10⁹ to about 10¹² TU/kg, or from about 10¹⁰ to about 10¹² TU/kg. Suitably, the lentiviral vector of the present invention is administered in a dose of from about 10⁸ to about 10¹¹ TU/kg, from about 10⁹ to about 10¹¹ TU/kg, or from about 10¹⁰ to about 10¹¹ TU/kg. In some embodiments, the lentiviral vector of the present invention is administered in a dose of from about 10⁸ to about 10¹¹ TU/kg, from about 10⁸ to about 10¹⁰ TU/kg, or from about 10⁹ to about 10¹⁰ TU/kg. In some embodiments, the lentiviral vector of the present invention is administered in a dose of from about 10⁹ to about 10¹⁰ TU/kg. The pharmaceutical composition may be formulated accordingly.

The pharmaceutical composition may further comprise one or more other therapeutic agents.

The invention further includes kits comprising the lentiviral vector, cell and/or pharmaceutical composition of the present invention. Preferably said kits are for use in the methods and used as described herein, e.g., the therapeutic methods as described herein. Preferably said kits comprise instructions for use of the kit components.

Methods for Treating and/or Preventing Disease

In one aspect, the present invention provides the lentiviral vector, cell or pharmaceutical composition according to the present invention for use as a medicament.

In one aspect, the present invention provides use of the lentiviral vector, cell or pharmaceutical composition according to the present invention in the manufacture of a medicament.

In one aspect, the present invention provides a method of administering a therapeutically effective amount of the lentiviral vector, cell or pharmaceutical composition according to the present invention to a subject in need thereof.

The lentiviral vector mediated gene therapy described herein may allow for a stable gene transfer even in paediatric patients at the first disease stages by virtue of lentiviral vector genomic integration.

Following administration of the lentiviral vector of the present invention, the lentiviral vector may integrate into the genome of liver cells (e.g. hepatocyte cells). Subsequently, the lentiviral vector may be maintained in the genome of liver cells (e.g. hepatocyte cells) as they duplicate. The integration of the lentiviral vector in the genome of liver cells may be determined by integration site (IS) analysis (e.g. quantitative high-throughput vector IS analysis). Suitable methods are known in the art (see e.g. Cantore, A., et al., 2015. Science translational medicine, 7(277), p.277ra28).

The lentiviral vector, cell or pharmaceutical composition may be administered to any subject in need thereof. The subject may be a mammal (e.g. a human). In some embodiments, the subject is male. In some embodiments, the subject is female. In preferred embodiments, the lentiviral vector is administered in the form of a lentiviral particle.

In some embodiments, the subject is a juvenile, an adolescent, or a child. The term “juvenile” may refer to an individual that has not yet reached adulthood. The term “adolescent” may refer to an individual during the period from the onset of puberty to adulthood. The term “child” may refer an individual between the stages of birth and puberty.

In some embodiments, the subject is a young child, a toddler, on an infant. The term “young child” may refer to a human subject aged from 3 years to 5 years. The term “toddler” may refer to a human subject aged from 1 year to 3 years. The term “infant” may mean refer to a human subject under the age of 12 months.

In some embodiments, the subject is a paediatric patient. The term “paediatric patient” may refer to a human subject until about 18-21 years of age (see e.g. Sawyer, S. M., et al., 2019. The Lancet Child & Adolescent Health, 3(11), pp. 822-830).

In some embodiments, the subject is a neonatal patient or an infantile patient. The term “neonatal patient” may refer to a human subject who is aged about 4 weeks old or younger. The term “infantile patient” may refer to a human subject who is aged from about 4 weeks to about 1 year.

In other embodiments, the subject is an adult. Human liver is expected to completely renew every 5 years in humans, so integrating vectors are expected to be more persisting compared to mostly episomal vectors (e.g. AAV).

The lentiviral vector mediated gene therapy described herein may allow for long-term treatment and/or prevention (e.g. following administration in a single, one off dose). For example, the vector mediated gene therapy described herein may be effective over a period of at least about 6 months or more, at least about 7 months or more, at least about 8 months or more, at least about 9 months or more, at least about 10 months or more, at least about 11 months or more, or at least about 1 year or more.

### Methylmalonic Acidemia (MMA)

The vector, cell or pharmaceutical composition according to the present invention may be used to prevent and/or treat methylmalonic acidemia (MMA).

In one aspect, the present invention provides the lentiviral vector, cell or pharmaceutical composition according to the present invention for use in preventing or treating methylmalonic acidemia (MMA).

In one aspect, the present invention provides use of the lentiviral vector, cell or pharmaceutical composition according to the present invention in the manufacture of a medicament for preventing or treating methylmalonic acidemia (MMA).

In one aspect, the present invention provides a method of preventing or treating methylmalonic acidemia (MMA), the method comprising administering a therapeutically effective amount of the lentiviral vector, cell or pharmaceutical composition according to the present invention to a subject in need thereof.

As described above, methylmalonic acidemia (MMA), also called methylmalonic aciduria, is a group of inborn errors of metabolism associated with elevated methylmalonic acid concentration in the blood and urine that result from the failure to convert methylmalonyl-CoA into succinyl-CoA, during propionyl-CoA metabolism in the mitochondrial matrix

In preferred embodiments, the MMA is isolated MMA (iMMA). As used herein, “isolated MMA” or “iMMA” may refer to MMA without hyperhomocysteinemia or homocystinuria, hypomethioninemia, or variations in other metabolites, such as malonic acid (see e.g. Manoli I, et al. 2005. Isolated Methylmalonic Acidemia. In GeneReviews).

MMA and iMMA may be sub-grouped according to the genetic defect, clinical presentation, and/or laboratory findings (see e.g. Hörster, F., et al., 2007. Pediatric research, 62(2), pp. 225-230; and Keyfi, F., et al., 2016. Reports of biochemistry & molecular biology, 5(1), pp. 1-14). Sub-types may include mut type MMA (mut-MMA), cblA type MMA (cblA-MMA), cblB type MMA (cblB-MMA), cblD type MMA (cblD-MMA), and MCEE deficiency.

In general, the mut forms of MMA is unresponsive to vitamin B12 therapy. Patients with defects in the synthesis of AdoCbl are usually responsive to vitamin B12 therapy and are classified as ‘cbl’ type. A definitive diagnosis of MMA may be based on urine organic acid analysis using gas chromatography/mass spectrometry. Patients with mut-MMA may have urine methylmalonic acid concentrations of from about 1000 to about 10000 mmol/mol creatinine; patients with cbl-MMA may have urine methylmalonic acid concentrations of from about 10 to about 100 mmol/mol creatinine; and/or patients with MCEE deficiency may have urine methylmalonic acid concentrations of from about 50 to about 1500 mmol/mol creatinine. MMA subtypes may be further diagnosed by enzyme assay analysis and/or molecular studies (see e.g. Keyfi, F., et al., 2016. Reports of biochemistry & molecular biology, 5(1), pp. 1-14).

In some embodiments, the MMA is selected from mut-MMA, cblA-MMA, cblB-MMA, cblD-MMA, and MCEE deficiency. In preferred embodiments, the MMA is mut-MMA.

Following administration of the lentiviral vector of the present invention to a subject in need thereof, plasma methylmalonic acid levels may be prevented from increasing or reduced. Suitably, plasma methylmalonic acid levels may be reduced by at least 10%, at least 20%, at least 30%, at least 40%, or at least 50%. Suitably, following administration, the subject's plasma methylmalonic acid level may be less than about 500 μmol/L, or less than about 200 μmol/L. Following administration of the lentiviral vector of the present invention to a subject in need thereof, plasma methylmalonic acid levels may be normalised. A normal plasma methylmalonic acid level may be <0.27 μmol/L (see e.g. Manoli I, et al. 2005. Isolated Methylmalonic Acidemia. In GeneReviews). Plasma methylmalonic acid levels may be determined by any method known in the art, for example as described in Rizzo, C., et al., 2014. Clinica chimica acta, 429, pp. 30-33.

Following administration of the lentiviral vector of the present invention to a subject in need thereof, urine methylmalonic acid levels may be prevented from increasing or reduced. Suitably, urine methylmalonic acid levels may be reduced by at least 10%, at least 20%, at least 30%, at least 40%, or at least 50%. Suitably, following administration, the subject's urine methylmalonic acid level may be less than about 5000 mmol/mol Cr, less than about 2000 mmol/mol Cr, or less than about 1000 mmol/mol Cr. Following administration of the lentiviral vector of the present invention to a subject in need thereof, urine methylmalonic acid levels may be normalised. A normal urine methylmalonic acid level may be <4 mmol/mol Cr (see e.g. Manoli I, et al. 2005. Isolated Methylmalonic Acidemia. In GeneReviews). Urine methylmalonic acid levels may be determined by any method known in the art, for example as described in Rasmussen, K., 1989. Clinical chemistry, 35(2), pp. 260-264.

Following administration of the lentiviral vector of the present invention to a subject in need thereof, liver methylmalonic acid levels may be prevented from increasing or reduced. Suitably, liver methylmalonic acid levels may be reduced by at least 10%, at least 20%, at least 30%, at least 40%, or at least 50%. Suitably, following administration, the subject's liver methylmalonic acid level may be less than about 50 μmol/L, less than about 10 μmol/L, or less than about 5 μmol/L. Following administration of the lentiviral vector of the present invention to a subject in need thereof, liver methylmalonic acid levels may be normalised. Liver methylmalonic acid levels may be determined by any method known in the art, for example as disclosed herein.

Following administration of the lentiviral vector of the present invention to a subject in need thereof, kidney methylmalonic acid levels may be prevented from increasing or reduced. Suitably, kidney methylmalonic acid levels may be reduced by at least 10%, at least 20%, at least 30%, at least 40%, or at least 50%. Suitably, following administration, the subject's kidney methylmalonic acid level may be less than about 100 μmol/L, or less than about 50 μmol/L. Following administration of the lentiviral vector of the present invention to a subject in need thereof, kidney methylmalonic acid levels may be normalised. Kidney methylmalonic acid levels may be determined by any method known in the art, for example as disclosed herein.

Following administration of the lentiviral vector of the present invention to a subject in need thereof, brain methylmalonic acid levels may be prevented from increasing or reduced. Suitably, brain methylmalonic acid levels may be reduced by at least 10%, at least 20%, at least 30%, at least 40%, or at least 50%. Suitably, following administration, the subject's brain methylmalonic acid level may be less than about 25 μmol/L, or less than about 20 μmol/L. Following administration of the lentiviral vector of the present invention to a subject in need thereof, brain methylmalonic acid levels may be normalised. Brain methylmalonic acid levels may be determined by any method known in the art, for example as disclosed herein.

Following administration of the lentiviral vector of the present invention to a subject in need thereof the mitochondrial function of the subject may be improved. Mitochondrial function may be determined by any suitable method known in the art, for example by measuring biochemical markers such as mitochondrial membrane potential, lactate, pyruvate, creatine kinase, acylcarnitine, and amino acids (e.g. alanine, glycine, proline and threonine), see e.g. Hubens, W. H. G., et al., 2021. Mitochondrion, 62, pp. 187-204. Suitably, following administration of the lentiviral vector of the present invention to a subject in need thereof, plasma FGF21 levels may be prevented from increasing or reduced. Suitably, plasma FGF21 levels may be reduced by at least 10%, at least 20%, at least 30%, at least 40%, or at least 50%. Suitably, following administration, the subject's plasma FGF21 levels may be less than about 1000 μg/ml. Following administration of the lentiviral vector of the present invention to a subject in need thereof, plasma FGF21 levels may be normalised. A normal plasma FGF21 level may be <200 μg/ml. Plasma FGF21 levels may be determined by any method known in the art, for example as described in Manoli, I., et al., 2018. JCI insight, 3(23), e124351.

Following administration of the lentiviral vector of the present invention to a subject in need thereof hepatic mitochondriopathy, renal mitochondriopathy, and/or hepatorenal mitochondriopathy may be ameliorated. Following administration of the lentiviral vector of the present invention to a subject in need thereof megamitochondria formation (e.g. in the liver) may be reduced and/or prevented. The progression of hepatic, renal, and/or hepatorenal mitochondriopathy and/or the formation of megamitochondria may be determined by any suitable method known in the art (see e.g. Chandler, R. J., et al., 2009. The FASEB Journal, 23(4), pp. 1252-1261), for example, by electron microscopy analysis. Suitably, following administration of the lentiviral vector of the present invention to a subject in need thereof, the mitochondrial area, perimeter and/or shape of mitochondria in the liver is normalised (e.g. any difference compared to a phenotypically normal subject may be statistically insignificant). Suitably, following administration of the lentiviral vector of the present invention to a subject in need thereof, the number of ring-shaped structures in mitochondria in the kidney is normalised (e.g. any difference compared to a phenotypically normal subject may be statistically insignificant).

Following administration of the lentiviral vector of the present invention to a subject in need thereof glutathione deficiency (e.g. in the liver) may be reduced and/or prevented. Following administration of the lentiviral vector of the present invention to a subject in need thereof, glutathione levels (e.g. hepatic glutathione levels) may be normalised. Glutathione levels may be determined by any suitable method known in the art (see e.g. Chandler, R. J., et al., 2009. The FASEB Journal, 23(4), pp. 1252-1261).

Following administration of the lentiviral vector of the present invention to a subject in need thereof poor growth may be reduced and/or prevented. Following administration of the lentiviral vector of the present invention to a subject in need thereof, growth may be normalised. Growth may be determined by any suitable method known in the art, e.g. using weight-for-age z-score and/or height-for-age z-scores.

mut type MMA (mut-MMA)

mut-MMA (MCID: MTH076, OMIM: 251000) is also called methylmalonic aciduria due to methylmalonyl-CoA mutase deficiency and is caused by a variety of mutations in MMUT, the gene coding for mitochondrial methylmalonyl-CoA mutase.

mut-MMA may be caused by a complete (mut⁰ enzymatic subtype) deficiency or a partial (mut⁻ enzymatic subtype) deficiency of the enzyme methylmalonyl-CoA mutase encoded by MMUT. OH-Cbl responsiveness may be used to differentiate between mut⁰ and mut⁻ subtypes (see e.g. Hörster, F., et al., 2007. Pediatric research, 62(2), pp. 225-230). For example, cells of patients showing an at least 1.5-fold increase in propionate incorporation after administration of OH-Cbl may be classified as mut⁻, whereas those with less or no increase may be classified as mut°.

In some embodiments, the MMA is mut-MMA and the MMA-associated polypeptide is MMUT, or fragment and/or variant thereof. The mut-MMA may be mut⁰ subtype or mut⁻ subtype. In some embodiments, the mut-MMA is mut⁰ subtype.

In one aspect, the present invention provides the lentiviral vector, cell or pharmaceutical composition according to the present invention for use in preventing or treating mut-MMA.

In one aspect, the present invention provides use of the lentiviral vector, cell or pharmaceutical composition according to the present invention in the manufacture of a medicament for preventing or treating mut-MMA.

In one aspect, the present invention provides a method of preventing or treating mut-MMA, the method comprising administering a therapeutically effective amount of the lentiviral vector, cell or pharmaceutical composition according to the present invention to a subject in need thereof.

cblA Type MMA (cblA-MMA)

cblA-MMA (MCID: MTH077, OMIM: 251100) is also called methylmalonic aciduria, CblA type and is caused by a variety of mutations in MMAA, the gene coding for mitochondrial methylmalonic aciduria type A protein.

In some embodiments, the MMA is cblA-MMA and the MMA-associated polypeptide is MMAA, or a fragment and/or variant thereof.

In one aspect, the present invention provides the lentiviral vector, cell or pharmaceutical composition according to the present invention for use in preventing or treating cblA-MMA.

In one aspect, the present invention provides use of the lentiviral vector, cell or pharmaceutical composition according to the present invention in the manufacture of a medicament for preventing or treating cblA-MMA.

In one aspect, the present invention provides a method of preventing or treating cblA-MMA, the method comprising administering a therapeutically effective amount of the lentiviral vector, cell or pharmaceutical composition according to the present invention to a subject in need thereof.

cblB Type MMA (cblB-MMA)

cblB-MMA (MCID: MTH078, OMIM: 251110) is also called methylmalonic aciduria, CblB type and is caused by a variety of mutations in MMAB, the gene coding for mitochondrial methylmalonic aciduria type B protein.

In some embodiments, the MMA is cblB-MMA and the MMA-associated polypeptide is MMAB, or a fragment and/or variant thereof.

In one aspect, the present invention provides the lentiviral vector, cell or pharmaceutical composition according to the present invention for use in preventing or treating cblB-MMA.

In one aspect, the present invention provides use of the lentiviral vector, cell or pharmaceutical composition according to the present invention in the manufacture of a medicament for preventing or treating cblB-MMA.

In one aspect, the present invention provides a method of preventing or treating cblB-MMA, the method comprising administering a therapeutically effective amount of the lentiviral vector, cell or pharmaceutical composition according to the present invention to a subject in need thereof.

cblD Type MMA (cblD-MMA)

cblD-MMA (MCID: MTH055, OMIM: 277410) is also called methylmalonic aciduria and homocystinuria, cblD type and is caused by a variety of mutations in MMADHC, the gene coding for mitochondrial methylmalonic aciduria and homocystinuria type D protein.

In some embodiments, the MMA is cblD-MMA and the MMA-associated polypeptide is MMADHC, or a fragment and/or variant thereof.

In one aspect, the present invention provides the lentiviral vector, cell or pharmaceutical composition according to the present invention for use in preventing or treating cblD-MMA.

In one aspect, the present invention provides use of the lentiviral vector, cell or pharmaceutical composition according to the present invention in the manufacture of a medicament for preventing or treating cblD-MMA.

In one aspect, the present invention provides a method of preventing or treating cblD-MMA, the method comprising administering a therapeutically effective amount of the lentiviral vector, cell or pharmaceutical composition according to the present invention to a subject in need thereof.

### MCEE Deficiency

MCEE deficiency (MCID: MTH040, OMIM: 251120) is also called methylmalonyl-CoA epimerase deficiency and is caused by a variety of mutations in MCEE, the gene coding for mitochondrial methylmalonyl-CoA epimerase.

In some embodiments, the MMA is MCEE deficiency and the MMA-associated polypeptide is MCEE, or a fragment and/or variant thereof.

In one aspect, the present invention provides the lentiviral vector, cell or pharmaceutical composition according to the present invention for use in preventing or treating MCEE deficiency.

In one aspect, the present invention provides use of the lentiviral vector, cell or pharmaceutical composition according to the present invention in the manufacture of a medicament for preventing or treating MCEE deficiency.

In one aspect, the present invention provides a method of preventing or treating MCEE deficiency, the method comprising administering a therapeutically effective amount of the lentiviral vector, cell or pharmaceutical composition according to the present invention to a subject in need thereof.

### EXAMPLES

Preferred features and embodiments of the invention will now be described by way of non-limiting examples.

### Example 1—Lentiviral Vector (LV) Encoding a MUT Transgene

We generated and produced a lentiviral vector (LV) encoding a MUT transgene and evaluated in vitro in hepatocyte cell lines and in vivo both in wild-type (WT) mice and in a mouse model of MMA, the Mut^{-/-};Tg^{INS-MCK-Mut} mice (MCK-Mut −/−).

### Results

We generated and produced LV encoding a human wild type or codon optimized MUT transgene under the control of a hepatocyte-specific cassette based on the enhanced transthyretin promoter (ET.MUT) or the human alpha-1 antitrypsin promoter (hAAT.MUT) (FIG. 1a).

To compare between the different MUT transgenes, we transduced Huh7 cells with LV-ET.MUTwt and LV-ET.MUTco_1 at increasing multiplicity of infection (MOI). We detected higher MUT expression in cells transduced with LV-ET.MUTco_1 compared to LV-ET.MUTwt, as detected by western blot analysis (FIG. 1b). The same result was obtained by transducing Huh7 cells with LV-hAAT.MUTwt or LV-hAAT.MUTco_1 at different MOI (FIG. 1c).

By MUT protein quantification and normalization on the GAPDH housekeeper, we confirmed a higher transgene expression (˜2 fold) obtained from the expression cassette driven by the ET promoter compared to the hAAT and by the codon optimized transgene compared to the wt form at similar vector DNA copies per cell (vector copy number, VCN) (FIG. 1d-f).

To directly compare the in vivo liver transduction efficiency of the LV-ET.MUTwt and LV-ET.MUTco, we administered them to 2-week old C57Bl/6 WT mice at a dose of 2.5×10¹⁰ transducing units (TU)/kg by i.v. injection. At the end of the experiment, 4 weeks post LV administration, the VCN in the liver, was ˜4 in the group injected with LV-ET.MUTwt and ˜2 in the group injected with LV-ET.MUTco (FIG. 2a). The MUT transgene expression was higher in the group injected with LV-ET.MUTco compared to LV-ET.MUTwt in terms of both mRNA expression (FIG. 2b) and protein output in the liver (FIG. 2c-d).

We performed a gene therapy experiment in MCK-Mut −/− mice, a model displaying clinical and biochemical features of MMA, including severe growth retardation, fragility, massively elevated serum MMA and hepatorenal mitochondrial disease (Manoli, I., et al., 2018. JCI insight, 3(23), e12435). The administration of LV-ET.MUTwt at a dose of 5×10¹⁰ TU/kg into juvenile MCK-Mut −/− mice resulted in 100% survival and complete normalization of their growth curve (FIG. 3a,b) and in a significant reduction in the circulating MMA levels until 12 months post-LV administration (FIG. 3c), thus indicating a substantial therapeutic benefit by LV-mediated liver gene therapy.

### Materials and Methods

### Plasmid Construction

The MUT coding sequences (WT or codon optimized) used in the study were synthetized by GeneScript and cloned into a third-generation self-inactivating (SIN) LV transfer plasmid (Milani, M., et al., 2019. Science Translational Medicine, 11(493), p.eaav7325) under the control of the enhanced transthyretin promoter (ET) or the human alpha-1 antitrypsin promoter (hAAT).

### Vector Production

Lab-grade VSV.G-pseudotyped third-generation SIN LV were produced by calcium phosphate transient transfection into 293T cells or 293T MHC-negative CD47-high, to obtain MHCfree CD47hi LV. Cells were transfected with a solution containing a mix of the selected LV genome transfer plasmid, the packaging plasmids pMDLg/pRRE and pCMV.REV, pMD2.G and pAdvantage, as previously described (Milani, M., et al., 2017. EMBO molecular medicine, 9(11), pp. 1558-1573; and Milani, M., et al., 2019. Science Translational Medicine, 11(493), p.eaav7325).

Medium was replaced 14-16 hours post transfection and supernatant was collected around 30 hours after medium change. LV-containing supernatants were sterilized through a 0.22 μm filter (Millipore) and transferred into sterile poliallomer tubes (Beckman) and centrifuged at 20,000 g for 120 min at 20° C. (Beckman Optima XL-100K Ultracentrifuge). LV pellet was resuspended in the appropriate volume of PBS to allow 500-1000× concentration.

### LV Titration

For LV titration, 1×10⁵ 293T cells were transduced with serial LV dilutions in the presence of polybrene (8 μg/ml). Genomic DNA (gDNA) was extracted 14 days after transduction, using Maxwell 16 Cell DNA Purification Kit (Promega), following manufacturer's instructions. VCN was determined was determined by ddPCR, starting from 5-20 ng of template gDNA using primers (HIV fw: 5′-T ACTGACGCTCTCGCACC-3′; HIV rv: 5′-TCTCGACGCAGGACTCG-3′) and a probe (FAM 5′-ATCTCTCTCCTTCTAGCCTC-3′) designed on the primer binding site region of LV. The amount of endogenous DNA was quantified by a primers/probe set designed on the human telomerase gene (Telo fw: 5′-GGCACACGTGGCTTTTCG-3′; Telo rv: 5′-GGTGAACCTCGTAAGTTTATGCAA-3′; Telo probe: VIC 5′-TCAGGACGTCGAGTGGACACGGTG-3′ TAMRA) or the human GAPDH gene (Applied Biosystems HS00483111_cm). The PCR reaction was performed with each primer (900 nM) and the probe (250 nM, 500 nM for Telo) following manufacturer's instructions (Biorad), read with QX200 reader and analyzed with QuantaSoft software (Biorad). Infectious titer, expressed as TU/mL, was calculated using the formula TU/mL=(VCN×100,000×(1/dilution factor). LV physical particles were measured by HIV-1 Gag p24 antigen immunocapture assay (Perkin Elmer) following manufacturer's instructions. LV specific infectivity was calculated as the ratio between infectious titer and physical particles.

### Cell Culture and In Vitro Transduction Experiments

HuH7 cells were maintained under 37° C., 5% CO₂ condition in Iscove's Modified Dulbecco's Medium (IMDM) supplemented with 10% FBS (Thermo Fisher Scientific, Waltham, MA). Cells were seeded into 12-well plates (2×10⁵ cells/well) and transduced with LV variants at different MOI in the presence of polybrene (8 μg/ml). Ten days post-LV transduction, cells were harvested.

### Mice Experiments

All animal experiments were performed in strict accordance with good animal practices following Italian and European legislation on animal care and experimentation (2010/63/EU). Wild-type C57Bl/6 or Mut^{-/-};Tg^{INS-MCK-Mut} 2-week old mice were used in these studies. Animals were administered with LV at a dose of 2.5×10¹⁰ TU/kg via the retro-orbital plexus. Blood samples were collected monthly throughout the duration of the study for analysis of the metabolites (see “Blood MMA determination”). For the experiment conducted in WT C57Bl/6 mice, animals were killed 4 weeks post LV administration and liver samples were collected and snap-frozen for vector copy number, mRNA and protein analyses.

### RNA Analysis

RNA samples were extracted from mouse livers using Maxwell 16 simplyRNA Tissue Kit (Promega). cDNA was synthetized starting from 1 μg of total RNA using the SuperScript IV VILO Master Mix (Invitrogen) according to manufacturer's instructions. LV gene expression was assessed by ddPCR starting from 25-50 ng of template cDNA using a primers/probe set designed on the WPRE region of LV (WPRE: primer fw 5′-GGCTGTTGGGCACTGACAAT-3′; primer rv 5′-ACGTCCCGCGCAGAATC-3′; probe FAM 5′-TTTCCTTGGCTGCTCGCCTGTGT-3′ NGB). Murine HPRT was used as reference gene (Bio Rad, Mmu 10031256 dMmu CPE5095493). The PCR reaction was performed with each primer (900 nM) and the probe (250 nM) following manufacturer's instructions (Biorad), read with QX200 reader and analyzed with QuantaSoft software (Biorad).

### Western Blot Analysis

Protein extracts from HuH7 cell or from mouse liver samples were prepared using RIPA buffer (EMD Millipore) and protease inhibitors (Roche). Protein concentration was determined using the DC Protein Assay (Bio Rad Laboratories). SDS-page electrophoresis was performed in a Bis-Tris 4-12% gradient polyacrylamide gel. After transfer, the membrane was blocked with 1×TBS+5% milk and incubated with an anti-MUT antibody (Abcam ab67869) or anti-actin (Sigma Aldrich A2228). The membrane was incubated with an anti-Mouse IgG secondary antibody (Jackson Immunoresearch 115-035-003), then with clarity Western ECL substrate (Bio Rad) and visualized by Uvitec Imaging System (Cleaver Scientific). For Western blot quantification, we used the Image J software.

### Plasmatic and Tissue MMA Determination

Blood samples were collected via the retro-orbital plexus in 0,5M EDTA-filled tubes. Dried blood spots (DBSs) were obtained from spotted blood on filter paper (903; Whatman GmbH, Dassel, Germany) after withdrawal of the samples. MMA measurement in DBSs was performed by Triple-Quad Mass Spectrometry, as previously described (Rizzo, C., et al., 2014. Clinica chimica acta, 429, pp. 30-33). Tissue MMA was determined in protein extracts from liver, kidney or brain samples and quantified by Triple-Quad Mass Spectrometry, as above.

### VCN Determination

DNA was extracted from cells or liver samples using Maxwell 16 Cell or Tissue DNA Purification Kits (Promega). VCN was determined in human macrophages as described above (see “LV titration”). VCN in murine DNA was determined by ddPCR, starting from 5-20 ng of template gDNA using a primers/probe set designed on the primer binding site region of LV (see “LV titration” above). The amount of endogenous murine DNA was quantified by a primers/probe set designed on the murine sema3a gene (Sema3A fw: 5′-ACCGATTCCAGATGATTGGC-3′; Sema3A rv: 5′-TCCATATTAATGCAGTGCTTGC-3′; Sema3A probe: HEX 5′-AGAGGCCTGTCCTGCAGCTCATGG-3′ BHQ1). The PCR reaction was performed with each primer (900 nM) and the probe (250 nM) following manufacturer's instructions (Biorad), read with QX200 reader and analyzed with QuantaSoft software (Biorad).

### Transmission Electron Microscopy (TEM) Analysis

For TEM analysis, tissues were fixed by needle injection perfusion as reported (Wisse E et al., World J Gastroenterol. 2010) with a fixative containing 2% formaldehyde, 2.5% glutaraldehyde in 0.15M NaCacodylate buffer pH 7.4. After the initial fixation samples were left in the same fixative at 4C.° overnight. Tissues were then chopped into 1 mm³ pieces and post-fixed in 1% osmium tetroxide, 1.5% potassium ferricyanide in 100 mM NaCacodylate buffer for 2 hours on ice. Samples were dehydrated with a crescent concentration of ethanol, washed with propylene oxide and infiltrated in a mixture of propylene oxide/epoxy resin overnight. The resin was then substituted with fresh epoxy resin and samples were embedded in silicone molds. Then, after being cured for 48 hours at 60C°, resin blocks were cut into ultrathin sections (70-90 nm) using an ultramicrotome (UC7, Leica microsystem, Vienna, Austria), collected on copper grids, stained with uranyl acetate and Sato's lead solutions and observed in a Transmission Electron Microscope Talos L120C (FEI, Thermo Fisher Scientific) operating at 120 kV. Images were acquired with a Ceta CCD camera ((FEI, Thermo Fisher Scientific).

### Liver Collection for Histopathology Analysis

Livers were harvested from mice and fixed in buffered formalin by immersion. Livers were trimmed, embedded in paraffin wax, sectioned, mounted on glass slides and stained with hematoxylin and eosin (H&E).

### Electron Microscopy Analysis of Mitochondria in Liver and Kidney

For transmission electron microscopy analysis tissues were fixed by needle injection perfusion (Wisse et al. (2010) World J Gastroenterol 16: 2851-66) with a fixative containing 2% formaldehyde, 2.5% glutaraldehyde in 0,15M NaCacodylate buffer pH 7.4. After the initial fixation samples were left in the same fixative at 4° C. overnight. Tissues were then chopped into 1 mm³ pieces and post-fixed in 1% osmium tetroxide, 1,5% potassium ferricyanide in 100 mM NaCacodylate buffer for 2 hours on ice. Samples were dehydrated with a crescent concentration of ethanol, washed with propylene oxide and infiltrated in a mixture of propylene oxide/epoxy resin overnight. The resin was then substituted with fresh epoxy resin and samples were embedded in silicone moulds. Then, after being cured for 48 hours at 60° C., resin blocks were cut into ultrathin sections (70-90 nm) using an ultramicrotome (UC7, Leica microsystem, Vienna, Austria), collected on copper grids, stained with uranyl acetate and Sato's lead solutions and observed in a Transmission Electron Microscope Talos L120C (FEI, Thermo Fisher Scientific) operating at 120 kV. Images were acquired with a Ceta CCD camera (FEI, Thermo Fisher Scientific). For each tissue area, corresponding to a single cell, 15 mitochondria were measured, 15 areas were measured, for a total of >200 mitochondria measured/mouse.

### Statistical Analysis

Statistical analyses were performed by Prism 9 software. Two-way ANOVA with Dunnet's multiple comparisons test was performed when comparing 2 or more independent groups over time.

### Example 2—Long Term Phenotypic Correction of Methylmalonic Acidemia

We administered 2-week old MCK-Mut −/− mice with LV.ET-MUT at a dose of 5E10 TU/kg and monitored them over a period of 12 months. LV treatment resulted in a complete rescue of the survival curve (FIG. 3a) and the normalization of the growth rate, comparable to that of heterozygous littermates (FIG. 3b). Importantly, we observed a rapid and significant decrease in circulating MMA compared to KO untreated controls, which was maintained long-term (1 year post LV) (FIG. 3c). At the end of the experiment, we measured the liver MMA, which was significantly reduced in MCK-Mut treated animals compared to KO untreated controls (FIG. 3d).

We confirmed the persistence of the vector and MUT transgene expression in the liver of treated mice 12 months post LV administration by vector copy number (VCN) and mRNA analyses (FIGS. 4a and b) and MUT protein expression in tissue lysates (FIG. 4c).

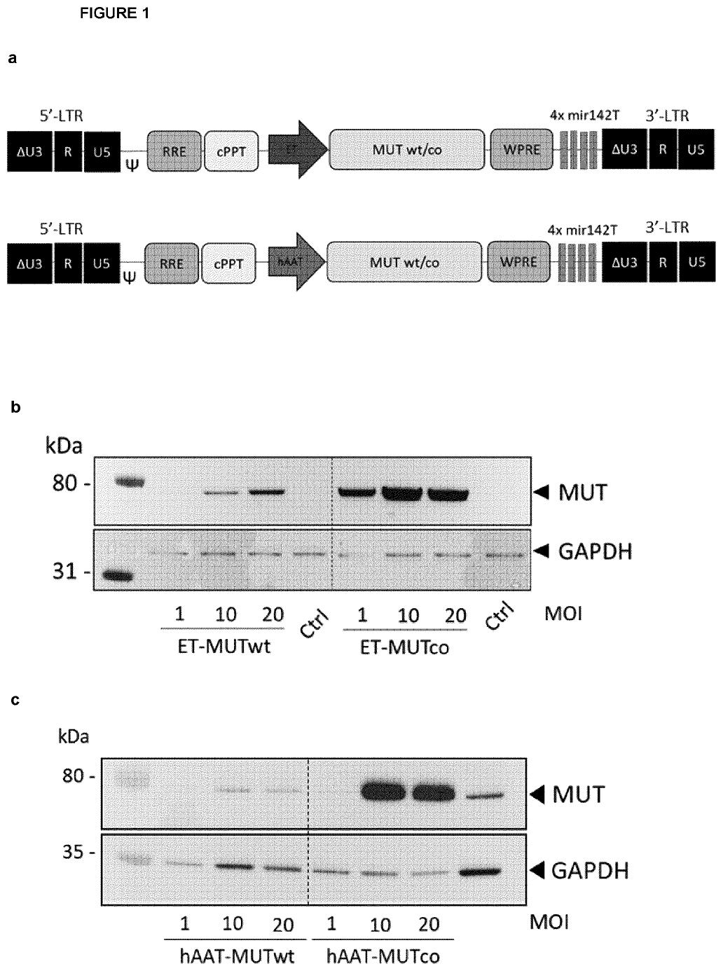

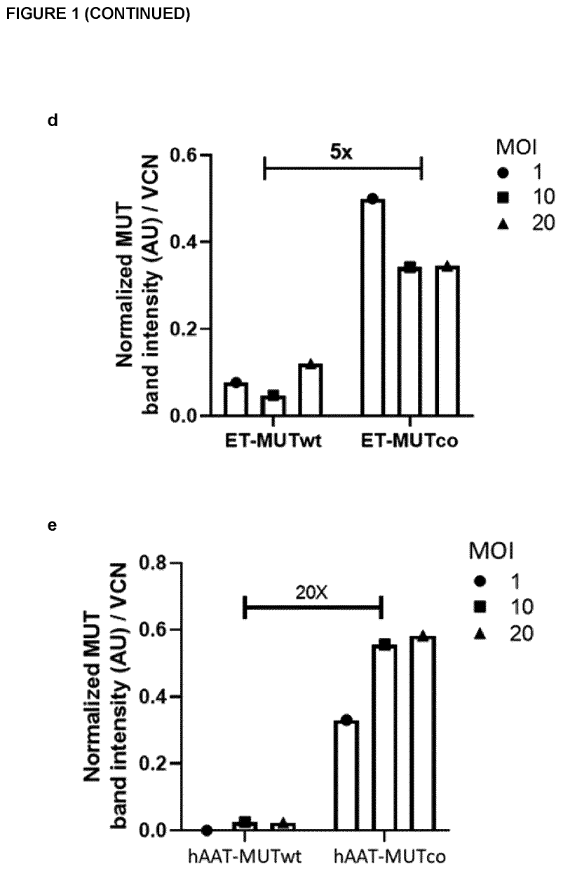

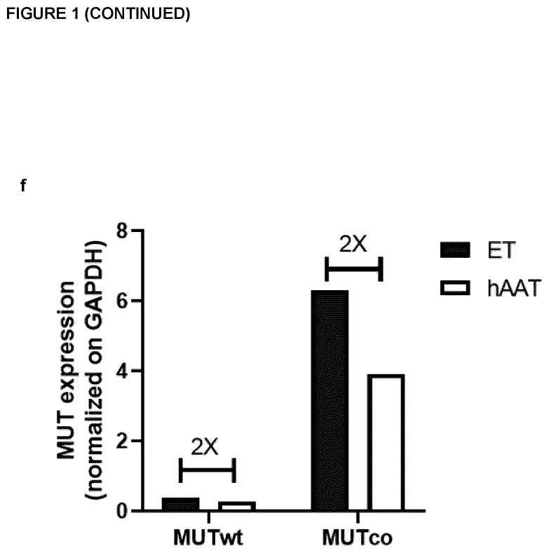

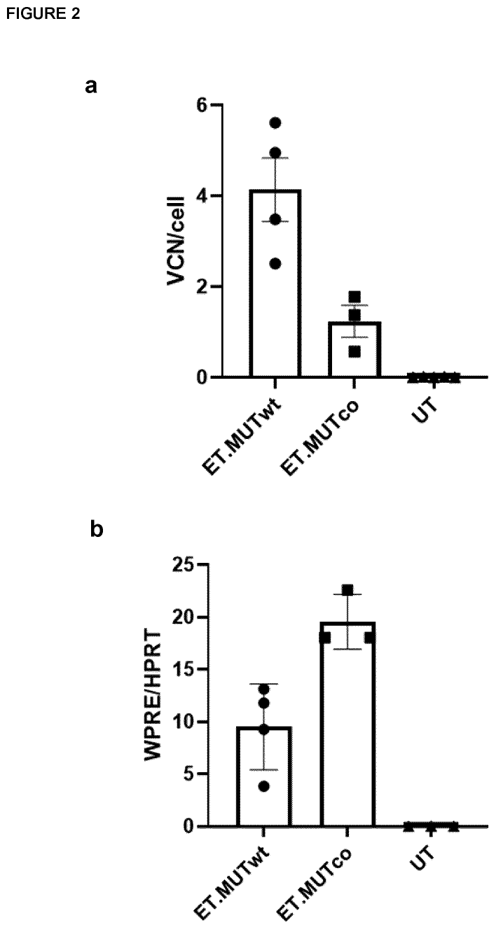

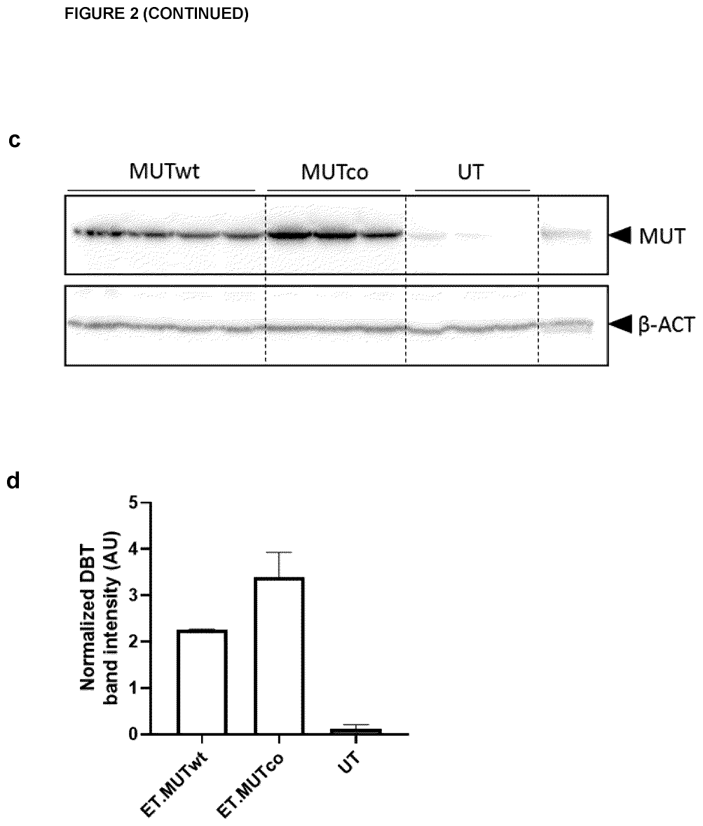

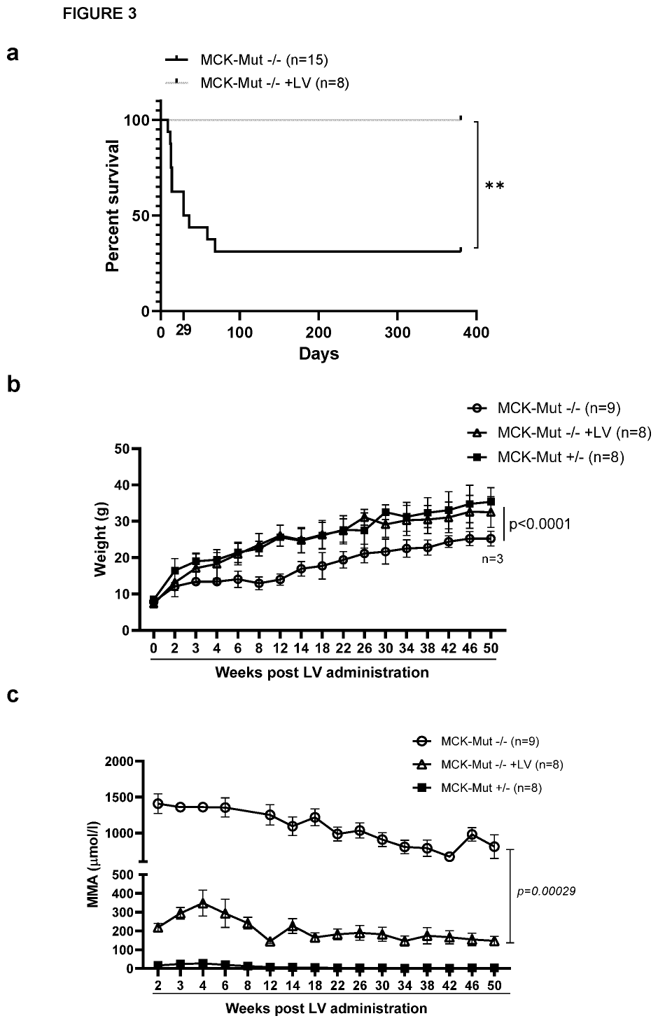

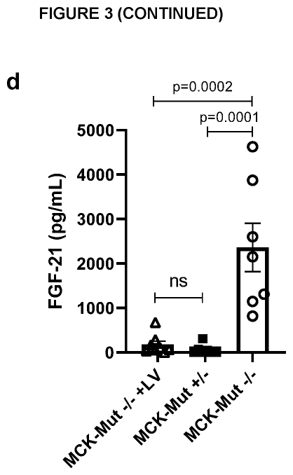

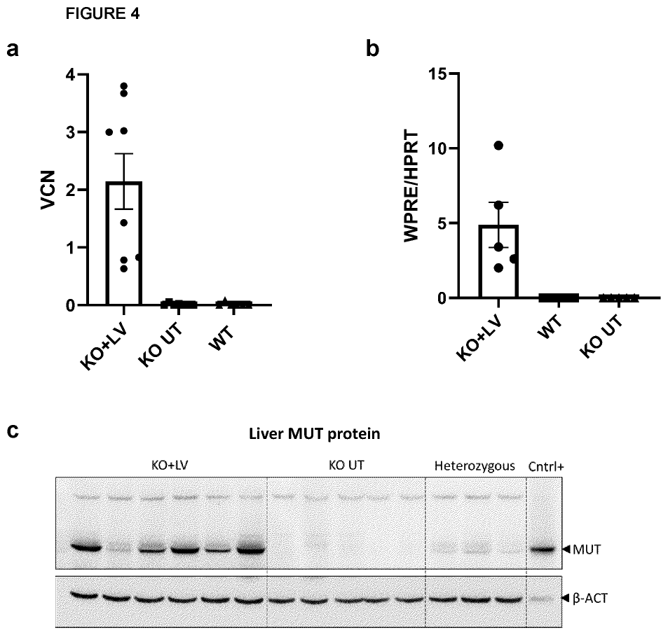

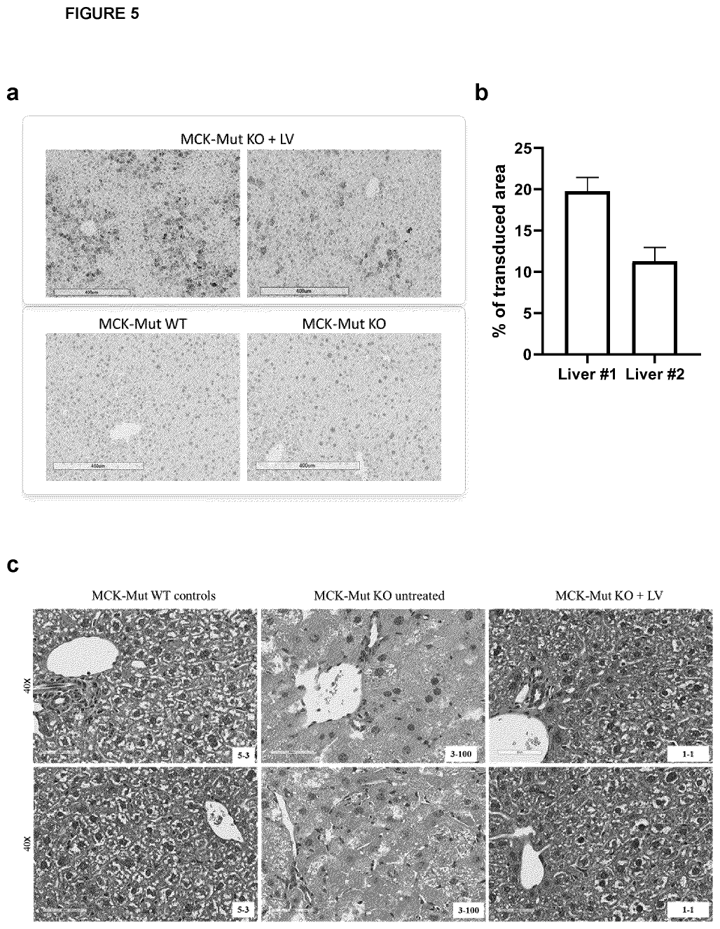

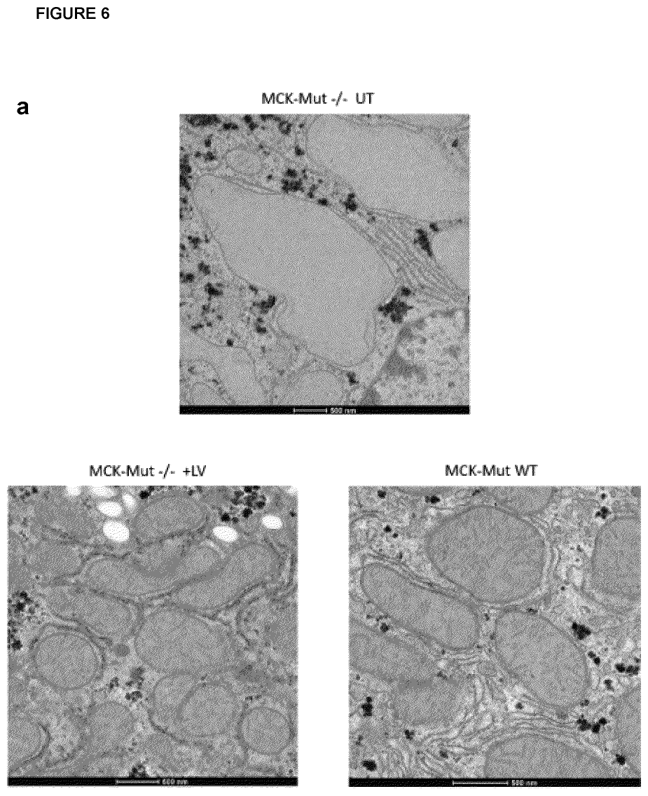

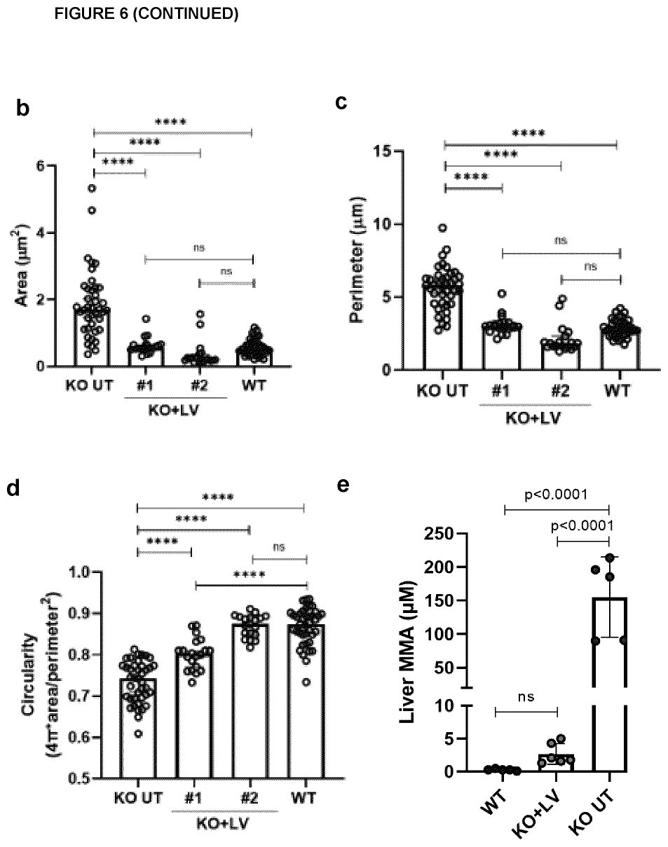

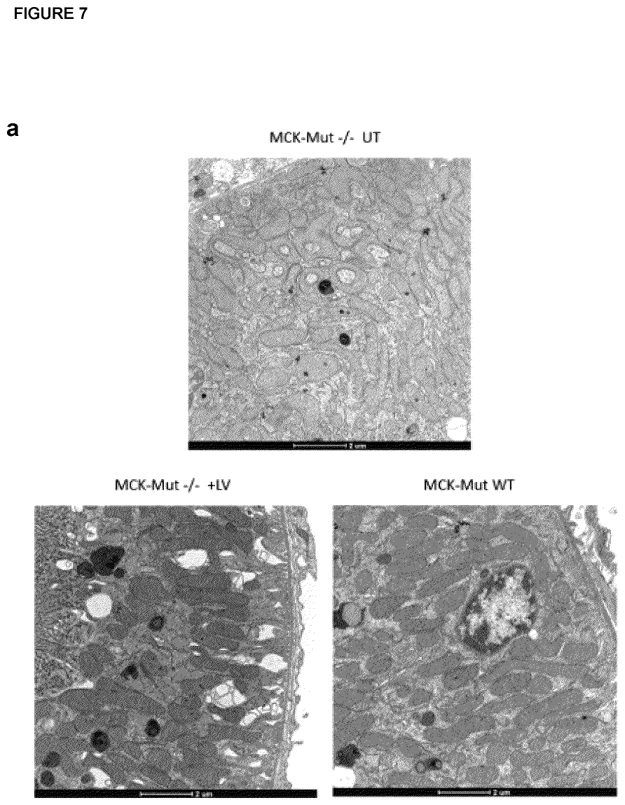

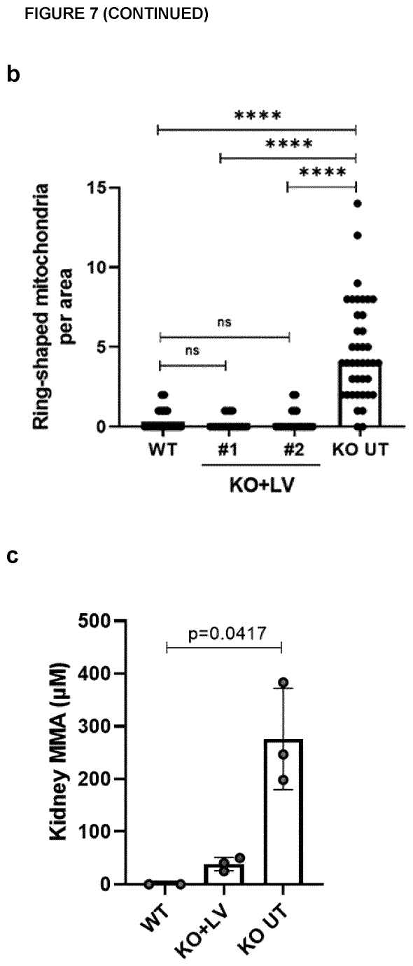

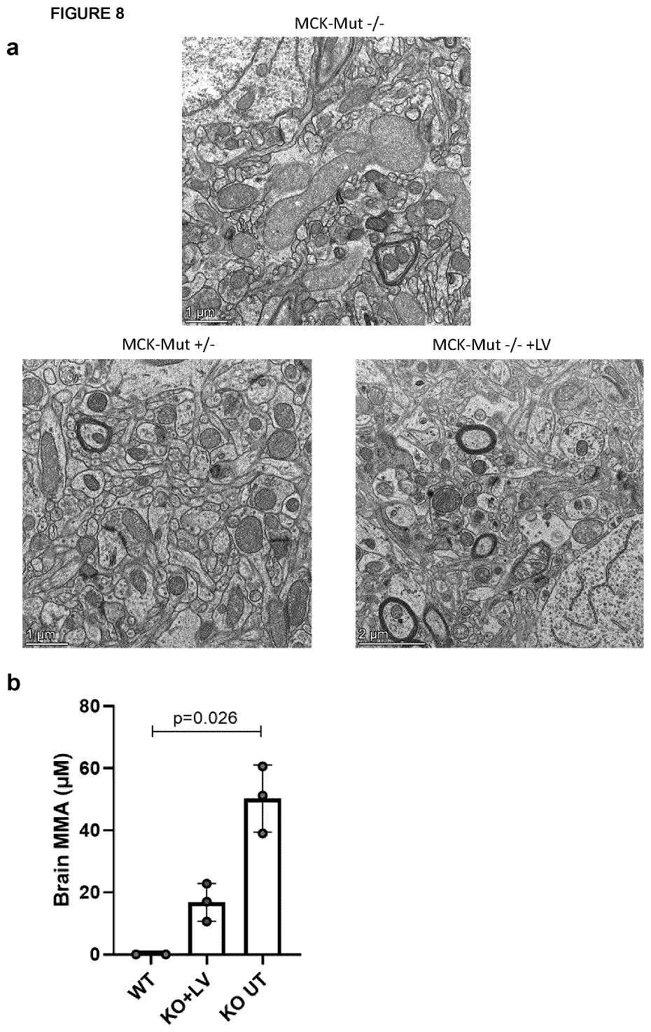

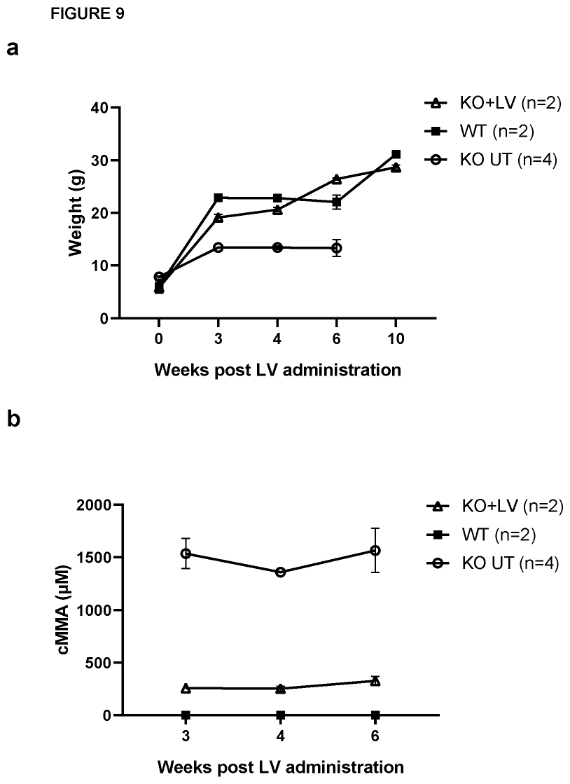

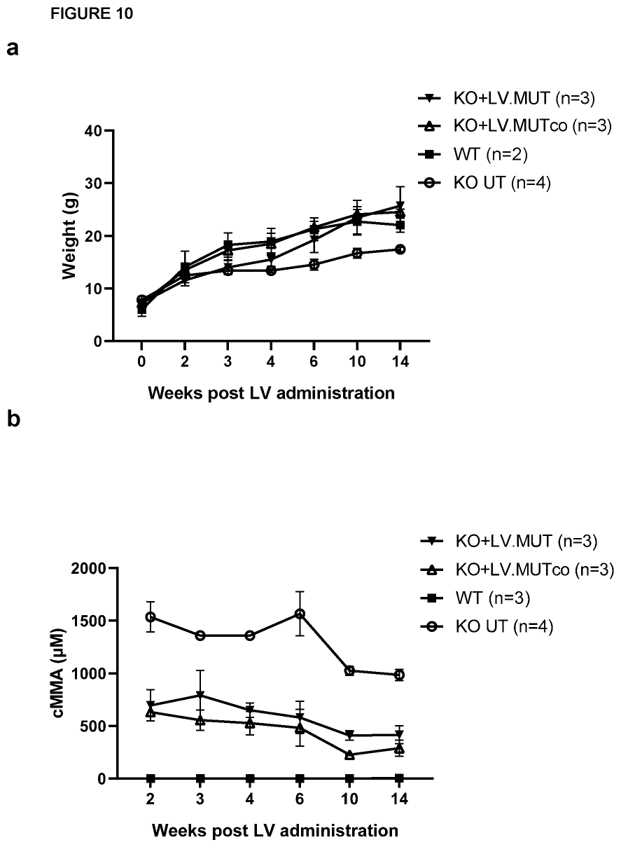

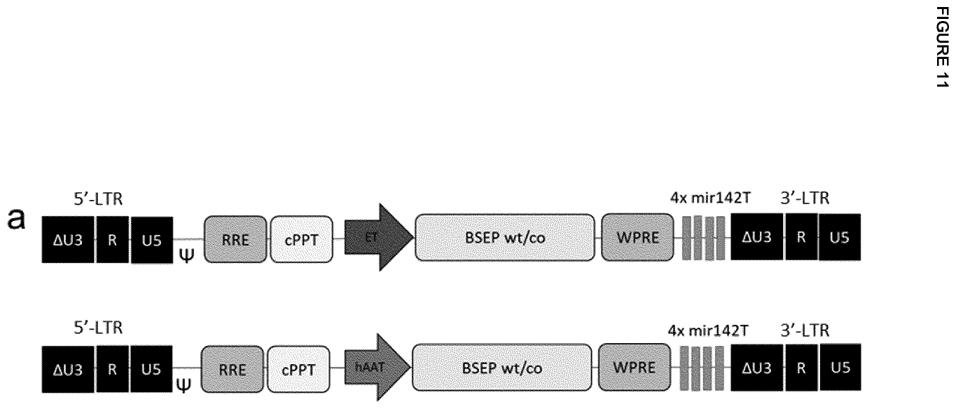

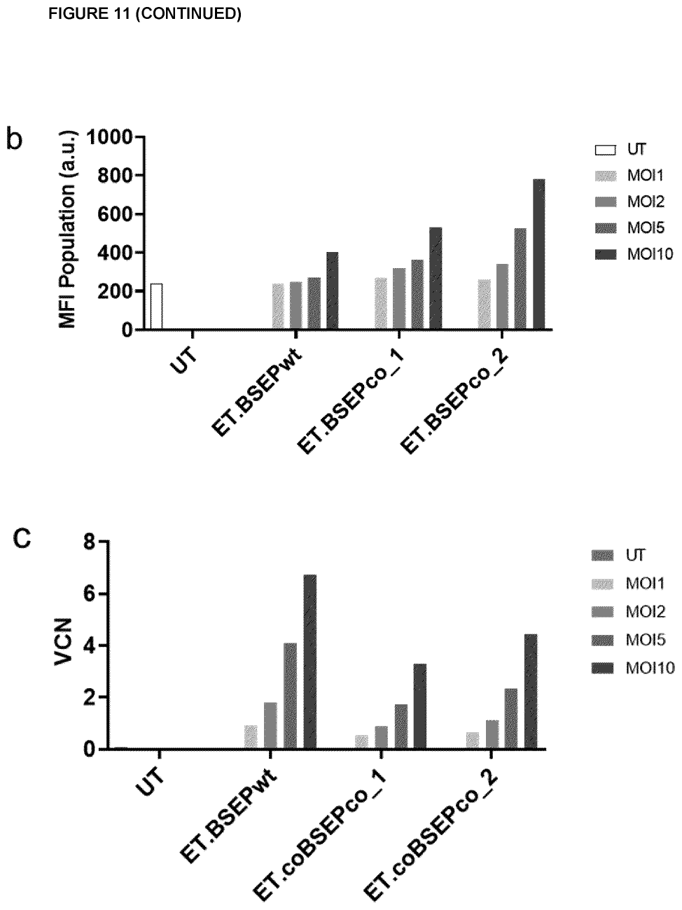

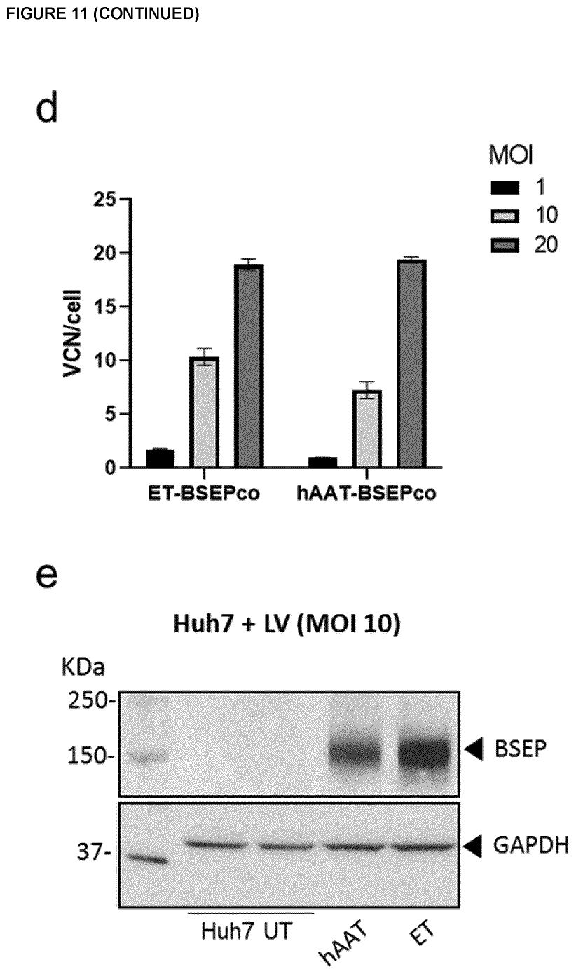

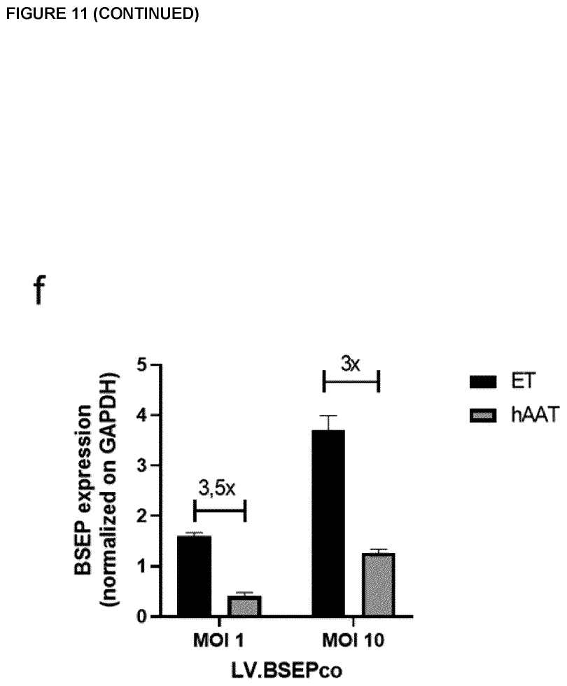

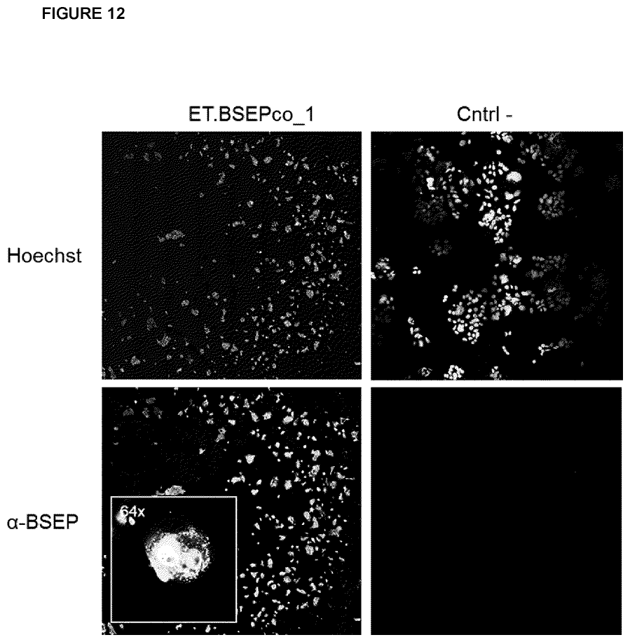

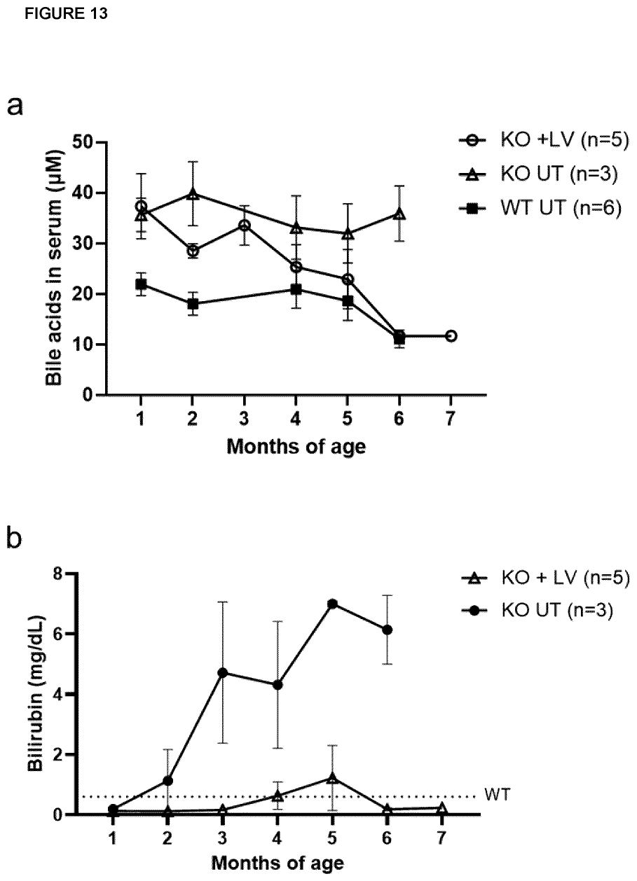

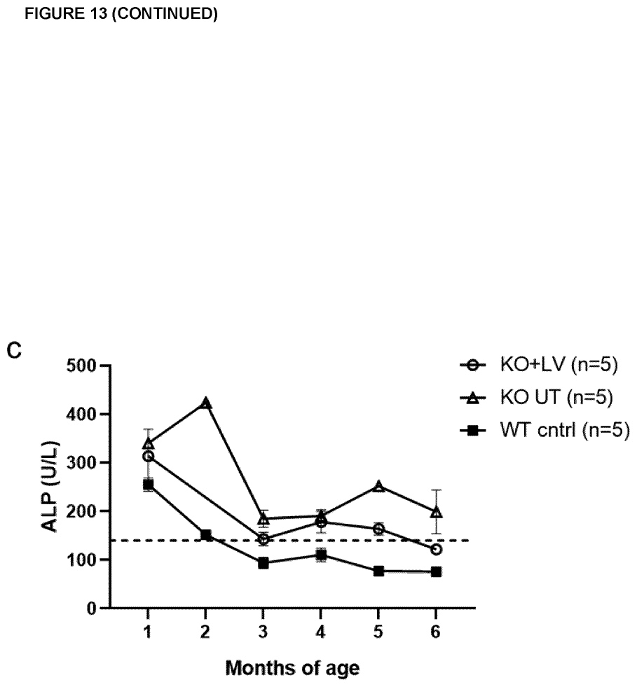

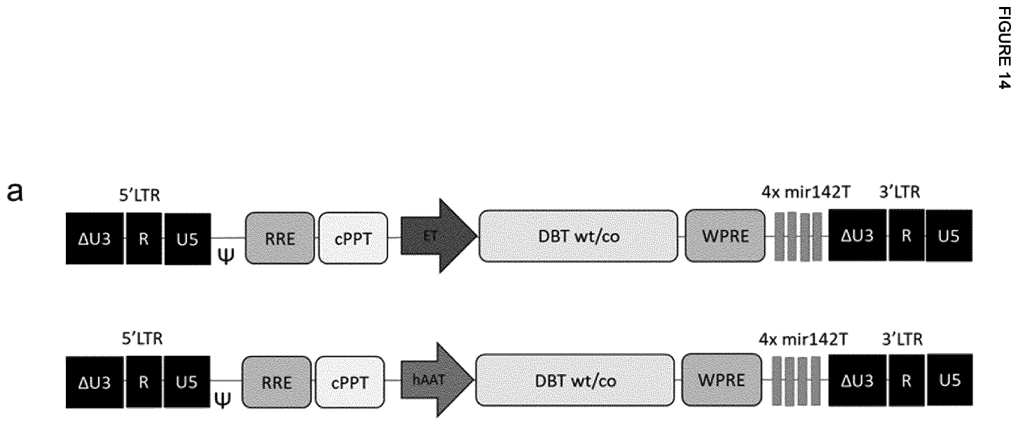

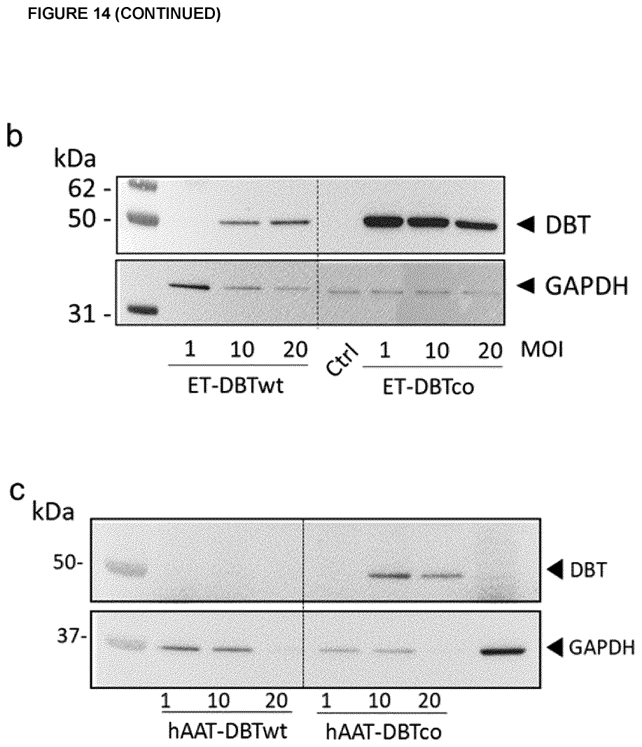

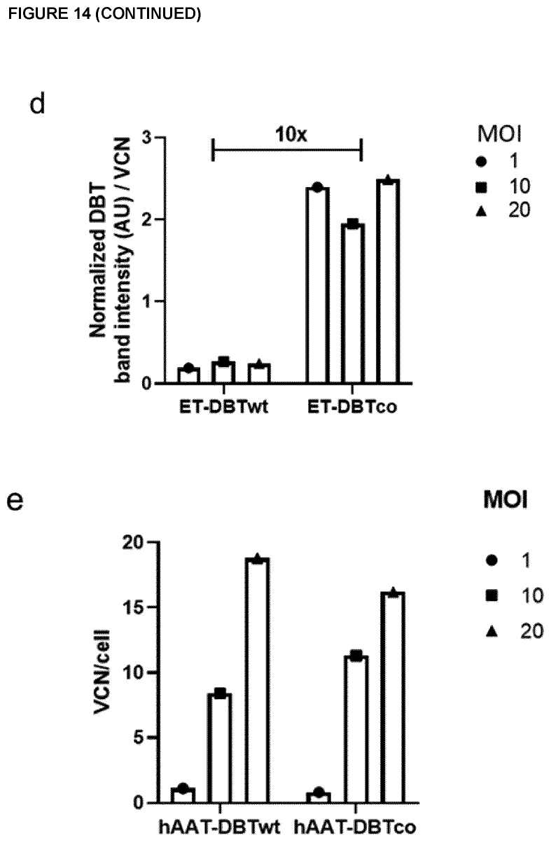

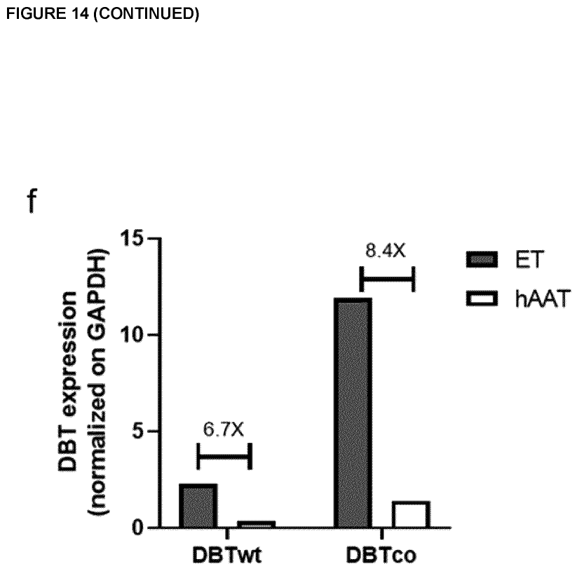

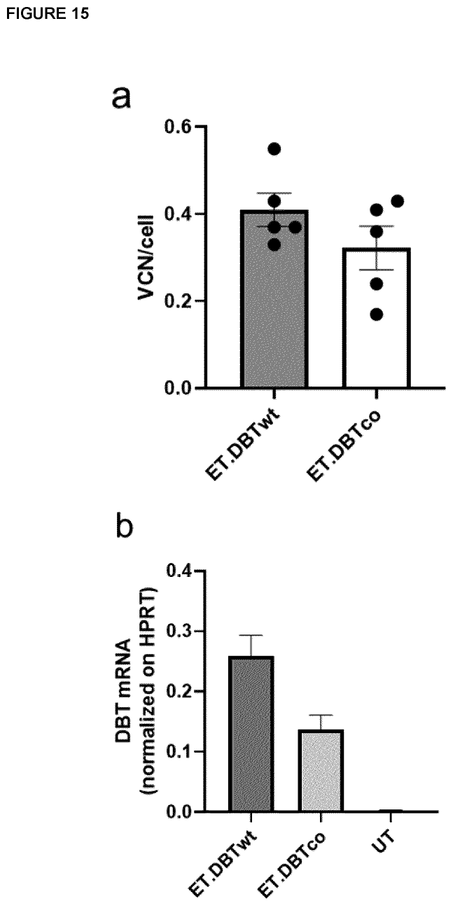

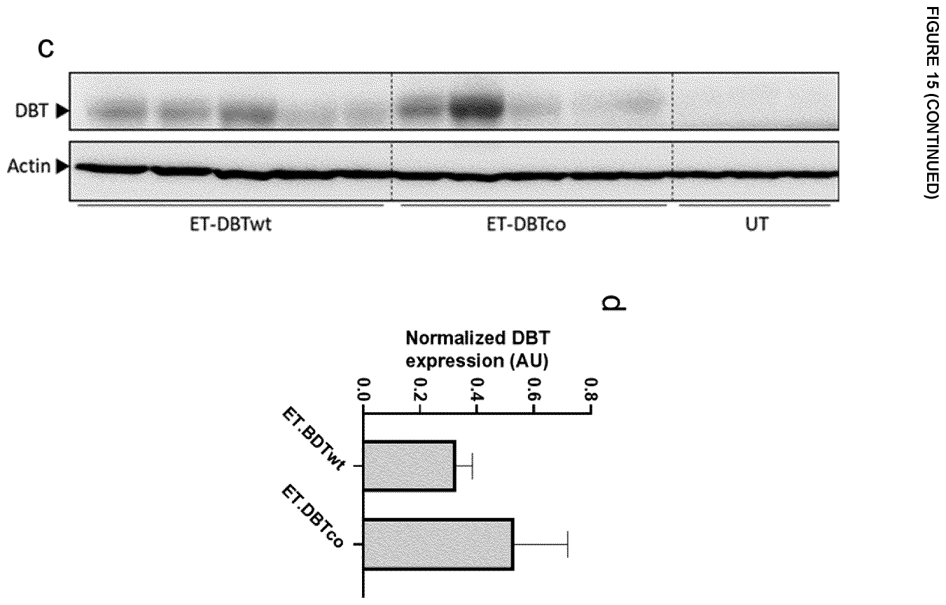

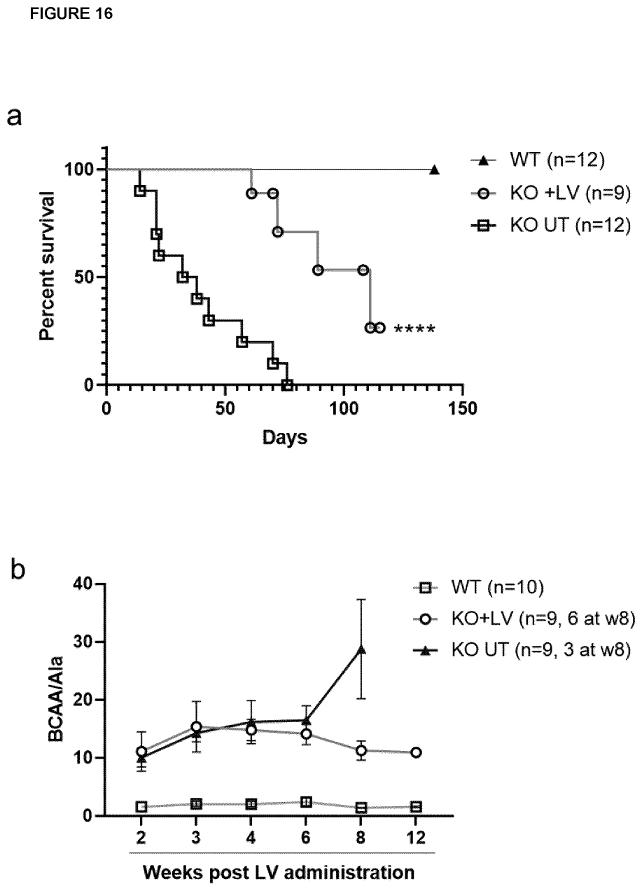

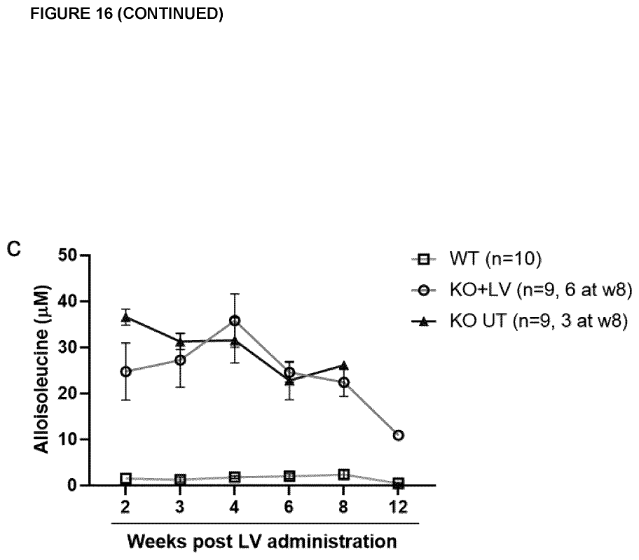

The liver transduction efficiency was quantified in two MCK-Mut treated mice by RNA ISH and resulted in around 10-20% of liver area (FIG. 5a-b). This data correlated with a global correction of the liver pathology of MCK-Mut mice, which were comparable to WT controls in terms of histopathological features (FIG. 5c).

Electron microscopy analysis of MCKMut −/− livers confirmed the presence of giant, abnormally shaped mitochondria with shortened or no cristae. LV treated mice showed a rescue of the mitochondrial structure, which was comparable to that of mitochondria of WT control animals (FIG. 6a). Image quantification showed a rescue of the mitochondrial area, perimeter and shape, measured as circularity index (FIGS. 6b-d). In line with the mitochondriopathy correction, the levels of toxic MMA in the liver were significantly reduced, almost normalized, compared to KO untreated controls (FIG. 6e).

In the proximal tubules of MCK-Mut−/− kidney, mitochondria appeared to be disorganized, ring-shaped and engulfed with cytosolic material. In LV treated MCK-Mut −/− mice, there was no evidence of renal mitochondriopathy (FIG. 7a). Image quantification showed that the number of ring-shaped structures was comparable to that of WT control mice (FIG. 7b). In line with the mitochondriopathy correction, the levels of toxic MMA in the kidney were significantly reduced compared to KO untreated controls (FIG. 7c).

Electron microscopy analysis of the cerebral cortex of MCK-Mut−/− showed some minor alteration of the mitochondrial structures. LV treated mice showed mitochondria ultrastructure comparable to that of WT control animals (FIG. 8a). In terms of MMA in the brain tissue, we showed accumulation of the toxic metabolite in MCK-Mut −/− untreated animals and a 3-fold decrease in brain MMA in LV treated mice, mediated by liver detoxification (FIG. 8b).

Administration of LV.ET-MUT to juvenile MCK-Mut −/− mice resulted in a complete rescue of the survival and growth curves and in a long-term significant reduction in circulating MMA levels, as well as liver, kidney and brain MMA, with amelioration of the hepatorenal mitochondriopathy.

Decrease of circulating MMA and rescue of the growth curve were confirmed in gene therapy experiments in which juvenile MCK-Mut −/− mice were administered with CD47^{high}-MCH^{free} LV.ET-MUT at a dose of 5E10 TU/kg (FIG. 9).

Administration of LV.ET-MUTco to juvenile MCK-Mut −/− mice resulted in similar or slightly improved rescue of the growth curve and reduction of circulating MMA compared to LV.ET.MUTwt at the same LV dose, 2.5E10 TU/kg (FIG. 10).

### Example 3—Lentiviral Vector (LV) Encoding a BSEP Transgene

We generated a lentiviral vector (LV) encoding a BSEP transgene and evaluated in vitro in hepatocyte cell lines and in vivo in a mouse model of PFIC-2, the Abcb11−/− mice.

### Results

We generated and produced LV encoding a human wild type or codon optimized BSEP transgenes under the control of a hepatocyte-specific cassette based on the enhanced transthyretin promoter (ET.BSEP) or the human alpha-1 antitrypsin promoter (hAAT.BSEP) (FIG. 11a).

To compare between the different BSEP transgenes, we first transduced Huh7 cells with LV-ET.BSEPwt, LV-ET.BSEPco_1 or LV-ET.BSEPco_2 at increasing multiplicity of infection (MOI). We performed flow cytometry analysis 10 days after transduction, detecting BSEP overexpression in LV-ET.BSEP transduced cells. Mean fluorescence intensity increased proportionally to the MOI employed and was higher for the BSEPco_1 and BSEPco_2 transgenes compared to BSEPwt at similar vector copy number (VCN) percell (FIG. 11b,c).

We then compared the activities of the ET and the hAAT promoters by transducing Huh7 cells with LV-ET.BSEPco_1 or LV-hAAT.BSEP_co1. VCN/cell was comparable in cells transduced with the two vectors at different MOI (FIG. 11d), while BSEP protein expression was higher in LV-ET.BSEPco_1 compared to LV-hAAT.BSEPco_1 transduced cells as detected by western blot analysis (FIG. 11e). By BSEP protein quantification and normalization on the GAPDH housekeeper, we confirmed a higher transgene expression (3-3.5 fold) obtained from the expression cassette driven by the ET promoter compared to the hAAT (FIG. 11f).

Immunofluorescence analysis on Huh7 cells transduced with LV-ET.BSEPco_1 confirmed proper BSEP membrane and cytoplasmic localization while no signal was detectable in Huh7 untransduced cells (FIG. 12).

We administered LV-ET.BSEPco_1 to juvenile Abcb11−/− mice at a dose of 3×10¹⁰ transducing units (TU)/kg by i.v. injection. The treatment resulted in a significant decrease of serum BA, bilirubin and ALP biomarkers until 7 months of age, indicating prevention of the progressive cholestatic damage (FIG. 13a-c).

### Materials and Methods

### Plasmid Construction

The BSEP coding sequences (WT or codon optimized) used in the study were synthetized by GeneScript and cloned into a third-generation self-inactivating (SIN) LV transfer plasmid (Milani, M., et al., 2019. Science Translational Medicine, 11(493), p.eaav7325) under the control of the enhanced transthyretin promoter (ET) or the human alpha-1 antitrypsin promoter (hAAT).

### Vector Production

Lab-grade VSV.G-pseudotyped third-generation SIN LV were produced by calcium phosphate transient transfection into 293T cells. 293T cells were transfected with a solution containing a mix of the selected LV genome transfer plasmid, the packaging plasmids pMDLg/pRRE and pCMV.REV, pMD2.G and pAdvantage, as previously described (Milani, M., et al., 2017. EMBO molecular medicine, 9(11), pp. 1558-1573; and Milani, M., et al., 2019. Science Translational Medicine, 11(493), p.eaav7325).

Medium was replaced 14-16 hours post transfection and supernatant was collected around 30 hours after medium change. LV-containing supernatants were sterilized through a 0.22 μm filter (Millipore) and transferred into sterile poliallomer tubes (Beckman) and centrifuged at 20,000 g for 120 min at 20° C. (Beckman Optima XL-100K Ultracentrifuge). LV pellet was resuspended in the appropriate volume of PBS to allow 500-1000× concentration.

### LV Titration

For LV titration, 1×10⁵ 293T cells were transduced with serial LV dilutions in the presence of polybrene (8 μg/ml). Genomic DNA (gDNA) was extracted 10 days after transduction, using Maxwell 16 Cell DNA Purification Kit (Promega), following manufacturer's instructions. VCN was determined by digital droplet PCR (ddPCR) starting from 5-20 ng of template gDNA using primers (HIV fw: 5′-T ACTGACGCTCTCGCACC-3′; HIV rv: 5′-TCTCGACGCAGGACTCG-3′) and a probe (FAM 5′-ATCTCTCTCCTTCTAGCCTC-3′) designed on the primer binding site region of LV. The amount of endogenous DNA was quantified by a primers/probe set designed on the human GAPDH gene (Applied Biosystems HS00483111_cm). The PCR reaction was performed with each primer (900 nM) and the probe (250 nM) following manufacturer's instructions (Biorad), read with QX200 reader and analyzed with QuantaSoft software (Biorad). Infectious titer, expressed as TU/mL, was calculated using the formula TU/mL=(VCN×100,000×(1/dilution factor)). LV physical particles were measured by HIV-1 Gag p24 antigen immunocapture assay (Perkin Elmer) following manufacturer's instructions. LV specific infectivity was calculated as the ratio between infectious titer and physical particles.

### Cell Culture and In Vitro Transduction Experiments

HuH7 cells were maintained under 37° C., 5% CO₂ condition in Iscove's Modified Dulbecco's Medium (IMDM) supplemented with 10% FBS (Thermo Fisher Scientific, Waltham, MA). Cells were seeded into 12-well plates (2×10⁵ cells/well) and transduced with LV variants at different MOI in the presence of polybrene (8 μg/ml). Ten days post-LV transduction, cells were harvested for molecular analyses.

### Mice Experiments

All animal experiments were performed in strict accordance with good animal practices following Italian and European legislation on animal care and experimentation (2010/63/EU). Abcb11−/− mice (Jackson strain n.004125) 2-week old were used in these studies. Animals were administered with LV at a dose of 3×10¹⁰ TU/kg via the retro-orbital plexus. Blood samples were collected monthly throughout the duration of the study for analysis of the metabolites.

### VCN Determination

DNA was extracted from cells or liver samples using Maxwell 16 Cell or Tissue DNA Purification Kits (Promega). VCN was determined in Huh7 samples as described above (see “LV titration”). VCN in murine DNA was determined by ddPCR, starting from 5-20 ng of template gDNA using a primers/probe set designed on the primer binding site region of LV (see “LV titration” above). The amount of endogenous murine DNA was quantified by a primers/probe set designed on the murine sema3a gene (Sema3A fw: 5′-ACCGATTCCAGATGATTGGC-3′; Sema3A rv: 5′-TCCATATTAATGCAGTGCTTGC-3′; Sema3A probe: HEX 5′-AGAGGCCTGTCCTGCAGCTC

ATGG-3′ BHQ1). The PCR reaction was performed with each primer (900 nM) and the probe (250 nM) following manufacturer's instructions (Biorad), read with QX200 reader and analyzed with QuantaSoft software (Biorad).

### Western Blot Analysis

Protein extracts from HuH7 cell or from mouse liver samples were prepared using RIPA buffer (EMD Millipore) and protease inhibitors (Roche). Protein concentration was determined using the DC Protein Assay (Bio Rad Laboratories). SDS-page electrophoresis was performed in a Bis-Tris 4-12% gradient polyacrylamide gel. After transfer, the membrane was blocked with 1×TBS+5% milk and incubated with an anti-Abcb11 antibody (Sigma-Aldrich HPA019035) or anti-GAPDH (Biolegend 649201). The membrane was incubated with an anti-Mouse and an anti-Rabbit IgG secondary antibodies (Jackson Immunoresearch 115-035-003 and 111-035-144), then with clarity Western ECL substrate (Bio Rad) and visualized by Uvitec Imaging System (Cleaver Scientific). For Western blot quantification, we used the Image J software.

### BSEP Immunofluorescence Analysis

Huh7 LV-transduced cells were seeded in an 8-well slide (Nunc® Lab-Tek® Chamber Slide™ system) to reach 40-50% confluency. 24 hours later, cells were fixed in 4% PFA and permeabilized in PBS+Triton X-100 0.1% for 10 minutes at room temperature (RT). Cells were blocked in in PBST+1% BSA 1 hour at RT and stained with a rabbit anti-BSEP (ab255605, dil.1:100) primary antibody, overnight at 4° C. After an incubation with a goat anti rabbit IgG 488 secondary antibody (Invitrogen, dil.1:1000), the nuclear staining was performed with Hoechst (diluted at 2 ug/mL in PBS), 5 minutes at RT. The staining slide was acquired at Leica TCS SP8 SMD confocal microscope.

### Bile Acids, ALP, Bilirubin Determination

Total bile acids were determined in serum samples by using the Mouse Total Bile Acid Assay Kit (Crystal Chem, 80471) according to manufacturer's instructions. Serum ALP activity was measured with an International Federation of Clinical Chemistry and Laboratory Medicine optimized kinetic UV method in a SABA chemical analyzer (Seac-Radim), and it is expressed as units per liter (U/L).

### Example 4—Lentiviral Vector (LV) Encoding a DBT Transgene

We generated and produced a lentiviral vector (LV) encoding a containing DBT transgene encoding the E2 subunit of BCKDC and evaluated in vitro in hepatocyte cell lines and in vivo both in wild-type (WT) mice and in an intermediate mouse model of MSUD due to E2 deficiency, the iMSUD mice.

### Results

We generated and produced LV carrying human wild type or codon optimized DBT transgenes under the control of a hepatocyte-specific cassette based on the enhanced transthyretin promoter (ET.DBT) or the human alpha-1 antitrypsin promoter (hAAT.DBT) (FIG. 14a).

To compare between the different DBT transgenes, we transduced Huh7 cells with LV-ET.DBTwt and LV-ET.DBTco_1 at increasing multiplicity of infection (MOI). We detected higher DBT expression in cells transduced with LV-ET.DBTco_1 compared to LV-ET.DBTwt, as detected by western blot analysis (FIG. 14b). The same result was obtained by transducing Huh7 cells with LV-hAAT.DBTwt or LV-hAAT.DBTco_1 at different MOI (FIG. 14c).

By DBT protein quantification and normalization on the GAPDH housekeeper, we confirmed a higher transgene expression (˜7-8 fold) obtained from the expression cassette driven by the ET promoter compared to the hAAT and by the codon optimized transgene compared to the wt form at similar vector DNA copies per cell (vector copy number, VCN) (FIG. 14d-f).

To directly compare the in vivo liver transduction efficiency of the LV-ET.DBTwt and LV-ET.DBTco, we administered them to juvenile C57Bl/6 WT mice at a dose of 2.5×10¹⁰ transducing units (TU)/kg by i.v. injection. At the end of the experiment, 4 weeks post LV administration, the vector copy number (VCN) in the liver, was ˜0.4 in both treated groups (FIG. 15a). The DBT mRNA was slightly higher in the group injected with LV-ET.DBTwt while the DBT protein output in the liver was slightly higher for the LV-ET.DBTco group (FIG. 15b-d).

We administered LV-ET.DBTco_1 to juvenile iMSUD mice at a dose of 5×10¹⁰ transducing units (TU)/kg by i.v. injection. The treatment resulted in an improved survival in treated mice compared to knock out littermates (50% survival probability at day 111 and 35, respectively) (FIG. 16a). The measurement of circulating BCAAs and alloisoleucine showed that these biomarkers remained stable in LV-treated animals up week 12 post LV (FIG. 16b,c). At the last time points, these parameters could not be evaluated in iMSUD untreated animals, which had previously died.

### Materials and Methods

### Plasmid Construction

The DBT coding sequences (WT or codon optimized) used in the study were synthetized by GeneScript and cloned into a third-generation self-inactivating (SIN) LV transfer plasmid (Milani, M., et al., 2019. Science Translational Medicine, 11(493), p.eaav7325) under the control of the enhanced transthyretin promoter (ET) or the human alpha-1 antitrypsin promoter (hAAT).

### Vector Production

Lab-grade VSV.G-pseudotyped third-generation SIN LV were produced by calcium phosphate transient transfection into 293T cells. 293T cells were transfected with a solution containing a mix of the selected LV genome transfer plasmid, the packaging plasmids pMDLg/pRRE and pCMV.REV, pMD2.G and pAdvantage, as previously described (Milani, M., et al., 2017. EMBO molecular medicine, 9(11), pp. 1558-1573; and Milani, M., et al., 2019. Science Translational Medicine, 11(493), p.eaav7325).

Medium was replaced 14-16 hours post transfection and supernatant was collected around 30 hours after medium change. LV-containing supernatants were sterilized through a 0.22 μm filter (Millipore) and transferred into sterile poliallomer tubes (Beckman) and centrifuged at 20,000 g for 120 min at 20° C. (Beckman Optima XL-100K Ultracentrifuge). LV pellet was resuspended in the appropriate volume of PBS to allow 500-1000× concentration.

### LV Titration

For LV titration, 1×10⁵ 293T cells were transduced with serial LV dilutions in the presence of polybrene (8 μg/ml). Genomic DNA (gDNA) was extracted 10 days after transduction, using Maxwell 16 Cell DNA Purification Kit (Promega), following manufacturer's instructions. VCN was determined by digital droplet PCR (ddPCR) starting from 5-20 ng of template gDNA using primers (HIV fw: 5′-T ACTGACGCTCTCGCACC-3′; HIV rv: 5′-TCTCGACGCAGGACTCG-3′) and a probe (FAM 5′-ATCTCTCTCCTTCTAGCCTC-3′) designed on the primer binding site region of LV. The amount of endogenous DNA was quantified by a primers/probe set designed on the human GAPDH gene (Applied Biosystems HS00483111_cm). The PCR reaction was performed with each primer (900 nM) and the probe (250 nM) following manufacturer's instructions (Biorad), read with QX200 reader and analyzed with QuantaSoft software (Biorad). Infectious titer, expressed as TU/mL, was calculated using the formula TU/mL=(VCN×100000×(1/dilution factor)). LV physical particles were measured by HIV-1 Gag p24 antigen immunocapture assay (Perkin Elmer) following manufacturer's instructions. LV specific infectivity was calculated as the ratio between infectious titer and physical particles.

### Cell Culture and In Vitro Transduction Experiments

HuH7 cells were maintained under 37° C., 5% CO₂ condition in Iscove's Modified Dulbecco's Medium (IMDM) supplemented with 10% FBS (Thermo Fisher Scientific, Waltham, MA). Cells were seeded into 12-well plates (2×10⁵ cells/well) and transduced with LV variants at different MOI in the presence of polybrene (8 μg/ml). Ten days post-LV transduction, cells were harvested for molecular analyses.

### Mice Experiments

All animal experiments were performed in strict accordance with good animal practices following Italian and European legislation on animal care and experimentation (2010/63/EU). Wild-type C57Bl/6 or iMSUD juvenile mice were used in these studies. iMSUD is an intermediate MSUD mouse model, which is DBT KO and show low activity of BCKAD in the liver (Homanics, G. E., et al., 2006. BMC medical genetics, 7(1), pp. 1-13). Animals were administered with LV at a dose of 2.5-5×10¹⁰ TU/kg via the retro-orbital plexus. Blood samples were collected monthly throughout the duration of the study for analysis of the metabolites. For the experiment conducted in WT C57Bl/6 mice, animals were killed 4 weeks post LV administration and liver samples were collected and snap-frozen for vector copy number, mRNA and protein analyses.

### RNA Analysis

RNA samples were extracted from mouse livers using Maxwell 16 simplyRNA Tissue Kit (Promega). cDNA was synthetized starting from 1 μg of total RNA using the SuperScript IV VILO Master Mix (Invitrogen) according to manufacturer's instructions. LV gene expression was assessed by ddPCR starting from 25-50 ng of template cDNA using a primers/probe set designed on the WPRE region of LV (WPRE: primer fw 5′-GGCTGTTGGGCACTGACAAT-3′; primer rv 5′-ACGTCCCGCGCAGAATC-3′; probe FAM 5′-TTTCCTTGGCTGCTCGCCTGTGT-3′ NGB). Murine HPRT was used as reference gene (Bio Rad, Mmu 10031256 dMmu CPE5095493). The PCR reaction was performed with each primer (900 nM) and the probe (250 nM) following manufacturer's instructions (Biorad), read with QX200 reader and analyzed with QuantaSoft software (Biorad).

### VCN Determination

DNA was extracted from cells or liver samples using Maxwell 16 Cell or Tissue DNA Purification Kits (Promega). VCN was determined in Huh7 samples as described above (see “LV titration”). VCN in murine DNA was determined by ddPCR, starting from 5-20 ng of template gDNA using a primers/probe set designed on the primer binding site region of LV (see “LV titration” above). The amount of endogenous murine DNA was quantified by a primers/probe set designed on the murine sema3a gene (Sema3A fw: 5′-ACCGATTCCAGATGATTGGC-3′; Sema3A rv: 5′-TCCATATTAATGCAGTGCTTGC-3′; Sema3A probe: HEX 5′-AGAGGCCTGTCCTGCAGCTC

ATGG-3′ BHQ1). The PCR reaction was performed with each primer (900 nM) and the probe (250 nM) following manufacturer's instructions (Biorad), read with QX200 reader and analyzed with QuantaSoft software (Biorad).

### Western Blot Analysis

Protein extracts from HuH7 cell or from mouse liver samples were prepared using RIPA buffer (EMD Millipore) and protease inhibitors (Roche). Protein concentration was determined using the DC Protein Assay (Bio Rad Laboratories). SDS-page electrophoresis was performed in a Bis-Tris 4-12% gradient polyacrylamide gel. After transfer, the membrane was blocked with 1×TBS+5% milk and incubated with an anti-DBT antibody (Sigma-Aldrich HPA026481) or anti-β actin (Sigma Aldrich A2228). The membrane was incubated with an anti-Mouse and an anti-Rabbit IgG secondary antibodies (Jackson Immunoresearch 115-035-003 and 111-035-144), then with clarity Western ECL substrate (Bio Rad) and visualized by Uvitec Imaging System (Cleaver Scientific). For Western blot quantification, we used the Image J software.

### Blood BCAA and Alloisoleucine Determination

Blood samples were collected via the retro-orbital plexus in 0,5M EDTA-filled tubes. Dried blood spots (DBS) were obtained from spotted blood on filter paper (903; Whatman GmbH, Dassel, Germany) after withdrawal of the samples. BCAA and alloisoleucine measurement in DBS was performed by liquid chromatography-tandem mass spectrometry, as previously described.

### Statistical Analysis

Statistical analyses were performed by using Prism 9 software. Comparison of survival curves was performed by applying a Long-rank (Mantel-Cox) test.

### EMBODIMENTS

Various preferred features and embodiments of the present invention will now be described with reference to the following numbered paragraphs (paras).

1. A lentiviral vector comprising a nucleotide sequence encoding a methylmalonic acidemia (MMA)-associated polypeptide, optionally wherein the lentiviral vector is an immune-shielded lentiviral vector.

2. The lentiviral vector according to para 1, wherein the MMA-associated polypeptide is selected from methylmalonyl-CoA mutase (MMUT), or a fragment thereof; methylmalonic aciduria type A (MMAA), or a fragment thereof; methylmalonic aciduria type B (MMAB), or a fragment thereof; methylmalonic aciduria and homocystinuria type D (MMADHC), or a fragment thereof; or methylmalonyl-CoA epimerase (MCEE) or a fragment thereof.

3. The lentiviral vector according to para 1 or 2, wherein the MMA-associated polypeptide is MMUT, or a fragment thereof.

4. The lentiviral vector according to any preceding para, wherein the MMA-associated polypeptide comprises or consists of an amino acid sequence which is at least 70% identical to one of SEQ ID NOs: 37, 40, 42, 44 or 46, or a fragment thereof, preferably wherein the MMA-associated polypeptide comprises or consists of an amino acid sequence which is at least 70% identical to SEQ ID NO: 37 or a fragment thereof.

5. The lentiviral vector according to any preceding para, wherein the nucleotide sequence encoding a MMA-associated polypeptide comprises or consists of a nucleotide sequence which is at least 70% identical to one of SEQ ID NOs: 38, 39, 41, 43, 45 or 47, or a fragment thereof, preferably wherein the nucleotide sequence encoding a MMA-associated polypeptide comprises or consists of a nucleotide sequence which is at least 70% identical to one of SEQ ID NOs: 38 or 39, or a fragment thereof.

6. The lentiviral vector according to any preceding para, wherein the nucleotide sequence encoding a MMA-associated polypeptide is codon-optimised, preferably wherein the nucleotide sequence encoding a MMA-associated polypeptide comprises or consists of a nucleotide sequence which is at least 70% identical to SEQ ID NO: 39, or a fragment thereof.

7. The lentiviral vector according to any preceding para, wherein the lentiviral vector is a CD47^{high} lentiviral vector.

8. The lentiviral vector according to any preceding para, wherein the lentiviral vector is obtained from a CD47^{high} host cell, optionally wherein the host cell is genetically engineered to increase expression of CD47 on the cell surface.

9. The lentiviral vector according to any preceding para, wherein the lentiviral vector has at least about 2-fold more CD47 on its surface than a lentiviral vector obtained from an unmodified host cell.

10. The lentiviral vector according to any preceding para, wherein the lentiviral vector is a MHC-I^{free} lentiviral vector.

11. The lentiviral vector according to any preceding para, wherein the lentiviral vector is obtained from a MHC-I^{free} host cell, optionally wherein the host cell is genetically engineered to disrupt expression of MHC-I on the cell surface.

12. The lentiviral vector according to any preceding para, wherein MHC-I is not detectable on the surface of the lentiviral vector.

13. The lentiviral vector according to any preceding para, wherein the lentiviral vector is a CD47^{high}/MHC-I^{free} lentiviral vector.

14. The lentiviral vector according to any preceding para, wherein the lentiviral vector is obtained from a CD47^{high}/MHC-I^{free} host cell.

15. The lentiviral vector according to any preceding para, wherein the nucleotide sequence encoding a MMA-associated polypeptide is operably linked to one or more miRNA target sequences.

16. The lentiviral vector according to para 15, wherein the one or more miRNA target sequences suppress transgene expression in one or more cells other than hepatocytes, preferably wherein the one or more miRNA target sequence suppress transgene expression in hematopoietic-lineage cells and/or antigen-presenting cells.

17. The lentiviral vector according to para 15 or 16, wherein the one or more miRNA target sequences are selected from miR-181, miR-142, miR-223, and miR-155 target sequences.

18. The lentiviral vector according to any preceding para, wherein the nucleotide sequence encoding a MMA-associated polypeptide is operably linked to one or more mir-142 target sequence, two or more mir-142 target sequences, three or more mir-142 target sequences, or four or more mir-142 target sequences.

19. The lentiviral vector according to any preceding para, wherein the nucleotide sequence encoding a MMA-associated polypeptide is operably linked to four mir-142 target sequences.

20. The lentiviral vector according to any of paras 15-19, wherein the one or more miRNA target sequences comprise or consist of a nucleotide sequence which is at least 90% identical to SEQ ID NO: 17, preferably wherein the one or more miRNA target sequences comprise or consist of a nucleotide sequence which is at least 90% identical to SEQ ID NO: 18.

21. The lentiviral vector according to any preceding para, wherein the nucleotide sequence encoding a MMA-associated polypeptide is operably linked to a liver-specific promoter, preferably wherein the nucleotide sequence encoding a MMA-associated polypeptide is operably linked to a hepatocyte-specific promoter.

22. The lentiviral vector according to any preceding para, wherein the nucleotide sequence encoding a MMA-associated polypeptide is operably linked to a transthyretin (TTR) promoter, an alpha-1-antityrpsin (AAT) promoter, a thyroxine-binding globulin (TBG) promoter, a APoE/hAAT promoter, a HCR-hAAT promoter, a LP1 promoter, or a HLP promoter.

23. The lentiviral vector according to any preceding para, wherein the nucleotide sequence encoding a MMA-associated polypeptide is operably linked to a transthyretin (TTR) promoter, preferably wherein the nucleotide sequence encoding a MMA-associated polypeptide is operably linked to a Enh1mTTR (ET) promoter.

24. The lentiviral vector according to any preceding para, wherein the nucleotide sequence encoding a MMA-associated polypeptide is operably linked to a promoter which comprises or consists of a nucleotide sequence which is at least 70% identical to SEQ ID NO: 19.

25. The lentiviral vector according to any preceding para, wherein the lentiviral vector is pseudotyped, preferably wherein the lentiviral vector is VSV.G-pseudotyped.

26. The lentiviral vector according to any preceding para, wherein the lentiviral vector is a self-inactivating (SIN) lentiviral vector, preferably wherein the lentiviral vector comprises self-inactivating (SIN) LTRs which comprise or consist of a nucleotide sequence which is at least 70% identical to SEQ ID NO: 23, or a fragment thereof.

27. The lentiviral vector according to any preceding para, wherein the lentiviral vector is an integrating lentiviral vector and/or a replication-defective lentiviral vector.

28. The lentiviral vector according to any preceding para, wherein the lentiviral vector comprises a nucleotide sequence which is at least 70% identical to SEQ ID NO: 36.

29. An isolated cell comprising a lentiviral vector according to any of paras 1-28.

30. A pharmaceutical composition comprising a lentiviral vector according to any of paras 1-28, in combination with a pharmaceutically acceptable carrier, diluent or excipient.

31. The lentiviral vector according to any of paras 1-28 or pharmaceutical composition according to para 30, for use as a medicament.

32. Use of a lentiviral vector according to any of paras 1-28 or a pharmaceutical composition according to para 30, for the manufacture of a medicament.

33. A method comprising administering a therapeutically effective amount of a lentiviral vector according to any of paras 1-28 or a pharmaceutical composition according to para 30, to a subject in need thereof.

34. The lentiviral vector according to any of paras 1-28 or pharmaceutical composition according to para 30, for use in preventing or treating methylmalonic acidemia (MMA).

35. Use of a lentiviral vector according to any of paras 1-28 or a pharmaceutical composition according to para 30, for the manufacture of a medicament for preventing or treating methylmalonic acidemia (MMA).

36. A method of preventing or treating methylmalonic acidemia (MMA), comprising administering a therapeutically effective amount of a lentiviral vector according to any of paras 1-28 or a pharmaceutical composition according to para 30, to a subject in need thereof.

37. The lentiviral vector or pharmaceutical composition for use according to para 34, the use according to para 35, or the method according to para 36, wherein the MMA-associated polypeptide is MMUT, or a fragment thereof, and the MMA is mut type MMA (mut-MMA); wherein the MMA-associated polypeptide is MMAA, or a fragment thereof, and the MMA is cblA type MMA (cblA-MMA); wherein the MMA-associated polypeptide is MMAB, or a fragment thereof, and the MMA is cblB type MMA (cblB-MMA); wherein the MMA-associated polypeptide is MMADHC, or a fragment thereof, and the MMA is cblD type MMA (cblD-MMA); or wherein the MMA-associated polypeptide is MCEE, or a fragment thereof, and the MMA is due to MCEE deficiency.

38. The lentiviral vector or pharmaceutical composition for use according to para 34 or 37, the use according to para 35 or 37, or the method according to para 36 or 37, wherein the MMA-associated polypeptide is MMUT, or a fragment thereof, and the MMA is mut-MMA, preferably wherein the mut-MMA is mut⁰ subtype or mut⁻ subtype.

39. The lentiviral vector or pharmaceutical composition for use according to any of paras para 31, 34, 37-38, the use according to any of paras 32, 35, 37-38, or the method according to any of paras 33, 36-39, wherein the subject is a human subject.

40. The lentiviral vector or pharmaceutical composition for use according to any of paras para 31, 34, 37-39, the use according to any of paras 32, 35, 37-39, or the method according to any of paras 33, 36-39, wherein the subject is a juvenile.

41. The lentiviral vector or pharmaceutical composition for use according to any of paras para 31, 34, 37-39, the use according to any of paras 32, 35, 37-39, or the method according to any of paras 33, 36-39, wherein the subject is a paediatric patient, preferably wherein the subject is a neonatal patient or an infantile patient.

42. The lentiviral vector or pharmaceutical composition for use according to any of paras 31, 34, 37-41, the use according to any of paras 32, 35, 37-41, or the method according to any of paras 33, 36-41, wherein said lentiviral vector or said pharmaceutical composition is administered systemically, preferably wherein said lentiviral vector or said pharmaceutical composition is administered by intravenous injection or intraperitoneal injection.

43. The lentiviral vector or pharmaceutical composition for use according to any of paras 31, 34, 37-41, the use according to any of paras 32, 35, 37-41, or the method according to any of paras 33, 36-41, wherein said lentiviral vector or said pharmaceutical composition is administered locally, preferably wherein said lentiviral vector or said pharmaceutical composition is administered by direct injection, intra-arterial injection, or intraportal injection.

44. The lentiviral vector or pharmaceutical composition for use according to para 43, the use according to para 43, or the method according to para 43, wherein said lentiviral vector or said pharmaceutical composition is administered locally to the liver, preferably wherein said lentiviral vector or said pharmaceutical composition is administered by intrahepatic injection, intrahepatic arterial injection, or intraportal injection.

45. The lentiviral vector or pharmaceutical composition for use according to any of paras 31, 34, 37-44, the use according to any of paras 32, 35, 37-43, or the method according to any of paras 33, 36-44, wherein the lentiviral vector is administered at a dose of at least about 10⁸ TU/kg, at least about 10⁹ TU/kg, or at least about 10¹⁰ TU/kg.

46. The lentiviral vector or pharmaceutical composition for use according to any of paras 31, 34, 37-45, the use according to any of paras 32, 35, 37-45, or the method according to any of paras 33, 36-45, wherein the lentiviral vector is administered in a dose of from about 10⁸ to about 10¹¹ TU/kg, from about 10⁸ to about 10¹⁰ TU/kg, or from about 10⁹ to about 10¹⁰ TU/kg.

47. The lentiviral vector or pharmaceutical composition for use according to any of paras 31, 34, 37-46, the use according to any of paras 32, 35, 37-46, or the method according to any of paras 33, 36-46, wherein the lentiviral vector integrates into the genome of liver cells and is maintained as the liver cells duplicate, preferably wherein the lentiviral vector integrates into the genome of hepatocytes and is maintained as the hepatocytes duplicate.

48. The lentiviral vector or pharmaceutical composition for use according to any of paras 34, 37-47, the use according to any of paras 35, 37-47, or the method according to any of paras 36-47, wherein plasma methylmalonic acid levels and/or urine methylmalonic acid levels are reduced and/or normalised.

49. The lentiviral vector or pharmaceutical composition for use according to any of paras 34, 37-48, the use according to any of paras 35, 37-48, or the method according to any of paras 36-48, wherein mitochondrial function is improved, preferably wherein the plasma levels of Fgf21 are reduced.

All publications mentioned in the above specification are herein incorporated by reference. Various modifications and variations of the disclosed vectors, cells, compositions, kits and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. An immune-shielded lentiviral vector comprising a nucleotide sequence encoding a methylmalonic acidemia (MMA)-associated polypeptide.

2. The lentiviral vector according to claim 1, wherein the MMA-associated polypeptide is selected from methylmalonyl-CoA mutase (MMUT), or a fragment thereof; methylmalonic aciduria type A (MMAA), or a fragment thereof; methylmalonic aciduria type B (MMAB), or a fragment thereof; methylmalonic aciduria and homocystinuria type D (MMADHC), or a fragment thereof; or methylmalonyl-CoA epimerase (MCEE) or a fragment thereof.

3. The lentiviral vector according to claim 1 or 2, wherein the MMA-associated polypeptide is MMUT, or a fragment thereof.

4. The lentiviral vector according to any preceding claim, wherein the MMA-associated polypeptide comprises or consists of an amino acid sequence which is at least 70% identical to one of SEQ ID NOs: 37, 40, 42, 44 or 46, or a fragment thereof, preferably wherein the MMA-associated polypeptide comprises or consists of an amino acid sequence which is at least 70% identical to SEQ ID NO: 37 or a fragment thereof.

5. The lentiviral vector according to any preceding claim, wherein the nucleotide sequence encoding a MMA-associated polypeptide comprises or consists of a nucleotide sequence which is at least 70% identical to one of SEQ ID NOs: 38, 39, 41, 43, 45 or 47, or a fragment thereof, preferably wherein the nucleotide sequence encoding a MMA-associated polypeptide comprises or consists of a nucleotide sequence which is at least 70% identical to one of SEQ ID NOs: 38 or 39, or a fragment thereof.

6. The lentiviral vector according to any preceding claim, wherein the nucleotide sequence encoding a MMA-associated polypeptide is codon-optimised, preferably wherein the nucleotide sequence encoding a MMA-associated polypeptide comprises or consists of a nucleotide sequence which is at least 70% identical to SEQ ID NO: 39, or a fragment thereof.

7. The lentiviral vector according to any preceding claim, wherein the lentiviral vector is a CD47^{high} lentiviral vector.

8. The lentiviral vector according to any preceding claim, wherein the lentiviral vector is obtained from a CD47^{high} host cell, optionally wherein the host cell is genetically engineered to increase expression of CD47 on the cell surface.

9. The lentiviral vector according to any preceding claim, wherein the lentiviral vector has at least about 2-fold more CD47 on its surface than a lentiviral vector obtained from an unmodified host cell.

10. The lentiviral vector according to any preceding claim, wherein the lentiviral vector is a MHC-I^{free} lentiviral vector.

11. The lentiviral vector according to any preceding claim, wherein the lentiviral vector is obtained from a MHC-I^{free} host cell, optionally wherein the host cell is genetically engineered to disrupt expression of MHC-I on the cell surface.

12. The lentiviral vector according to any preceding claim, wherein MHC-I is not detectable on the surface of the lentiviral vector.

13. The lentiviral vector according to any preceding claim, wherein the lentiviral vector is a CD47^{high}/MHC-I^{free} lentiviral vector.

14. The lentiviral vector according to any preceding claim, wherein the lentiviral vector is obtained from a CD47^{high}/MHC-I^{free} host cell.

15. The lentiviral vector according to any preceding claim, wherein the nucleotide sequence encoding a MMA-associated polypeptide is operably linked to one or more miRNA target sequences.

16. The lentiviral vector according to claim 15, wherein the one or more miRNA target sequences suppress transgene expression in one or more cells other than hepatocytes, preferably wherein the one or more miRNA target sequence suppress transgene expression in hematopoietic-lineage cells and/or antigen-presenting cells.

17. The lentiviral vector according to claim 15 or 16, wherein the one or more miRNA target sequences are selected from miR-181, miR-142, miR-223, and miR-155 target sequences.

18. The lentiviral vector according to any preceding claim, wherein the nucleotide sequence encoding a MMA-associated polypeptide is operably linked to one or more mir-142 target sequence, two or more mir-142 target sequences, three or more mir-142 target sequences, or four or more mir-142 target sequences.

19. The lentiviral vector according to any preceding claim, wherein the nucleotide sequence encoding a MMA-associated polypeptide is operably linked to four mir-142 target sequences.

20. The lentiviral vector according to any of claims 15-19, wherein the one or more miRNA target sequences comprise or consist of a nucleotide sequence which is at least 90% identical to SEQ ID NO: 17, preferably wherein the one or more miRNA target sequences comprise or consist of a nucleotide sequence which is at least 90% identical to SEQ ID NO: 18.

21. The lentiviral vector according to any preceding claim, wherein the nucleotide sequence encoding a MMA-associated polypeptide is operably linked to a liver-specific promoter, preferably wherein the nucleotide sequence encoding a MMA-associated polypeptide is operably linked to a hepatocyte-specific promoter.

22. The lentiviral vector according to any preceding claim, wherein the nucleotide sequence encoding a MMA-associated polypeptide is operably linked to a transthyretin (TTR) promoter, an alpha-1-antityrpsin (AAT) promoter, a thyroxine-binding globulin (TBG) promoter, a APoE/hAAT promoter, a HCR-hAAT promoter, a LP1 promoter, or a HLP promoter.

23. The lentiviral vector according to any preceding claim, wherein the nucleotide sequence encoding a MMA-associated polypeptide is operably linked to a transthyretin (TTR) promoter, preferably wherein the nucleotide sequence encoding a MMA-associated polypeptide is operably linked to a Enh1mTTR (ET) promoter.

24. The lentiviral vector according to any preceding claim, wherein the nucleotide sequence encoding a MMA-associated polypeptide is operably linked to a promoter which comprises or consists of a nucleotide sequence which is at least 70% identical to SEQ ID NO: 19.

25. The lentiviral vector according to any preceding claim, wherein the lentiviral vector is pseudotyped, preferably wherein the lentiviral vector is VSV.G-pseudotyped.

26. The lentiviral vector according to any preceding claim, wherein the lentiviral vector is a self-inactivating (SIN) lentiviral vector, preferably wherein the lentiviral vector comprises self-inactivating (SIN) LTRs which comprise or consist of a nucleotide sequence which is at least 70% identical to SEQ ID NO: 23, or a fragment thereof.

27. The lentiviral vector according to any preceding claim, wherein the lentiviral vector is an integrating lentiviral vector and/or a replication-defective lentiviral vector.

28. The lentiviral vector according to any preceding claim, wherein the lentiviral vector comprises a nucleotide sequence which is at least 70% identical to SEQ ID NO: 36.

29. An isolated cell comprising a lentiviral vector according to any of claims 1-28.

30. A pharmaceutical composition comprising a lentiviral vector according to any of claims 1-28, in combination with a pharmaceutically acceptable carrier, diluent or excipient.

31. The lentiviral vector according to any of claims 1-28 or pharmaceutical composition according to claim 30, for use as a medicament.

32. Use of a lentiviral vector according to any of claims 1-28 or a pharmaceutical composition according to claim 30, for the manufacture of a medicament.

33. A method comprising administering a therapeutically effective amount of a lentiviral vector according to any of claims 1-28 or a pharmaceutical composition according to claim 30, to a subject in need thereof.

34. The lentiviral vector according to any of claims 1-28 or pharmaceutical composition according to claim 30, for use in preventing or treating methylmalonic acidemia (MMA).

35. Use of a lentiviral vector according to any of claims 1-28 or a pharmaceutical composition according to claim 30, for the manufacture of a medicament for preventing or treating methylmalonic acidemia (MMA).

36. A method of preventing or treating methylmalonic acidemia (MMA), comprising administering a therapeutically effective amount of a lentiviral vector according to any of claims 1-28 or a pharmaceutical composition according to claim 30, to a subject in need thereof.

37. The lentiviral vector or pharmaceutical composition for use according to claim 34, the use according to claim 35, or the method according to claim 36, wherein the MMA-associated polypeptide is MMUT, or a fragment thereof, and the MMA is mut type MMA (mut-MMA); wherein the MMA-associated polypeptide is MMAA, or a fragment thereof, and the MMA is cblA type MMA (cblA-MMA); wherein the MMA-associated polypeptide is MMAB, or a fragment thereof, and the MMA is cblB type MMA (cblB-MMA); wherein the MMA-associated polypeptide is MMADHC, or a fragment thereof, and the MMA is cblD type MMA (cblD-MMA); or wherein the MMA-associated polypeptide is MCEE, or a fragment thereof, and the MMA is due to MCEE deficiency.

38. The lentiviral vector or pharmaceutical composition for use according to claim 34 or 37, the use according to claim 35 or 37, or the method according to claim 36 or 37, wherein the MMA-associated polypeptide is MMUT, or a fragment thereof, and the MMA is mut-MMA, preferably wherein the mut-MMA is mut⁰ subtype or mut⁻ subtype.

39. The lentiviral vector or pharmaceutical composition for use according to any of claims claim 31, 34, 37-38, the use according to any of claims 32, 35, 37-38, or the method according to any of claims 33, 36-39, wherein the subject is a human subject.

40. The lentiviral vector or pharmaceutical composition for use according to any of claims claim 31, 34, 37-39, the use according to any of claims 32, 35, 37-39, or the method according to any of claims 33, 36-39, wherein the subject is a juvenile.

41. The lentiviral vector or pharmaceutical composition for use according to any of claims claim 31, 34, 37-39, the use according to any of claims 32, 35, 37-39, or the method according to any of claims 33, 36-39, wherein the subject is a paediatric patient, preferably wherein the subject is a neonatal patient or an infantile patient.

42. The lentiviral vector or pharmaceutical composition for use according to any of claims 31, 34, 37-41, the use according to any of claims 32, 35, 37-41, or the method according to any of claims 33, 36-41, wherein said lentiviral vector or said pharmaceutical composition is administered systemically, preferably wherein said lentiviral vector or said pharmaceutical composition is administered by intravenous injection or intraperitoneal injection.

43. The lentiviral vector or pharmaceutical composition for use according to any of claims 31, 34, 37-41, the use according to any of claims 32, 35, 37-41, or the method according to any of claims 33, 36-41, wherein said lentiviral vector or said pharmaceutical composition is administered locally, preferably wherein said lentiviral vector or said pharmaceutical composition is administered by direct injection, intra-arterial injection, or intraportal injection.

44. The lentiviral vector or pharmaceutical composition for use according to claim 43, the use according to claim 43, or the method according to claim 43, wherein said lentiviral vector or said pharmaceutical composition is administered locally to the liver, preferably wherein said lentiviral vector or said pharmaceutical composition is administered by intrahepatic injection, intrahepatic arterial injection, or intraportal injection.

45. The lentiviral vector or pharmaceutical composition for use according to any of claims 31, 34, 37-44, the use according to any of claims 32, 35, 37-43, or the method according to any of claims 33, 36-44, wherein the lentiviral vector is administered at a dose of at least about 10⁸ TU/kg, at least about 10⁹ TU/kg, or at least about 10¹⁰ TU/kg.

46. The lentiviral vector or pharmaceutical composition for use according to any of claims 31, 34, 37-45, the use according to any of claims 32, 35, 37-45, or the method according to any of claims 33, 36-45, wherein the lentiviral vector is administered in a dose of from about 10⁸ to about 10¹¹ TU/kg, from about 10⁸ to about 10¹⁰ TU/kg, or from about 10⁹ to about 10¹⁰ TU/kg.

47. The lentiviral vector or pharmaceutical composition for use according to any of claims 31, 34, 37-46, the use according to any of claims 32, 35, 37-46, or the method according to any of claims 33, 36-46, wherein the lentiviral vector integrates into the genome of liver cells and is maintained as the liver cells duplicate, preferably wherein the lentiviral vector integrates into the genome of hepatocytes and is maintained as the hepatocytes duplicate.

48. The lentiviral vector or pharmaceutical composition for use according to any of claims 34, 37-47, the use according to any of claims 35, 37-47, or the method according to any of claims 36-47, wherein plasma methylmalonic acid levels and/or urine methylmalonic acid levels are reduced and/or normalised.

49. The lentiviral vector or pharmaceutical composition for use according to any of claims 34, 37-48, the use according to any of claims 35, 37-48, or the method according to any of claims 36-48, wherein mitochondrial function is improved, preferably wherein the plasma levels of Fgf21 are reduced.

50. An immune-shielded lentiviral vector for use in a method of therapy, wherein the method comprises administration of the immune-shielded lentiviral vector to a juvenile or paediatric subject.

51. A cell for use in a method of therapy, wherein the cell comprises an immune-shielded lentiviral vector, and wherein the method comprises administration of the cell to a juvenile or paediatric subject.

52. The immune-shielded lentiviral vector or cell for use according to claim 50 or 51, wherein the subject is a neonatal subject or an infantile subject.


SEQUENCE LISTING (59 sequences)

SEQ ID NO: 1 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 2 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 292 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 4 - protein, 311 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 5 - protein, 287 aa, synthetic construct
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 6 - protein, 305 aa, synthetic construct
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 7 - protein, 274 aa, synthetic construct
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 8 - protein, 293 aa, synthetic construct
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 9 - protein, 140 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPN

SEQ ID NO: 10 - protein, 122 aa, synthetic construct
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPN

SEQ ID NO: 11 - protein, 365 aa, synthetic construct
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 12 - protein, 371 aa, synthetic construct
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSGGEGVKDRKGGSYTQAASSDSAQGSDVSLTACKV

SEQ ID NO: 13 - protein, 362 aa, synthetic construct
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 14 - protein, 366 aa, synthetic construct
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 15 - protein, 326 aa, synthetic construct
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEHLRSCTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLRWGGKGGSCSQAACSNSAQGSDESLITCKA

SEQ ID NO: 16 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 17 - DNA, 23 bp, synthetic construct
  source 1..23: mol_type = other DNA
tccataaagtaggaaacactaca

SEQ ID NO: 18 - DNA, 106 bp, synthetic construct
  source 1..106: mol_type = other DNA
tccataaagtaggaaacactacacgattccataaagtaggaaacactacaaccggttccataaagtaggaaacactacatcactccataaagtaggaaacactaca

SEQ ID NO: 19 - DNA, 553 bp, synthetic construct
  source 1..553: mol_type = other DNA
cgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 20 - DNA, 347 bp, synthetic construct
  source 1..347: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacag

SEQ ID NO: 21 - DNA, 504 bp, synthetic construct
  source 1..504: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgc

SEQ ID NO: 22 - skipped

SEQ ID NO: 23 - DNA, 235 bp, synthetic construct
  source 1..235: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 24 - DNA, 182 bp, synthetic construct
  source 1..182: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 25 - DNA, 235 bp, synthetic construct; identical to SEQ ID NO: 23
  source 1..235: mol_type = other DNA

SEQ ID NO: 26 - DNA, 154 bp, synthetic construct
  source 1..154: mol_type = other DNA
tggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagag

SEQ ID NO: 27 - DNA, 151 bp, synthetic construct
  source 1..151: mol_type = other DNA
tggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagag

SEQ ID NO: 28 - DNA, 365 bp, synthetic construct
  source 1..365: mol_type = other DNA
atgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgat

SEQ ID NO: 29 - DNA, 241 bp, synthetic construct
  source 1..241: mol_type = other DNA
ggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggattt

SEQ ID NO: 30 - DNA, 241 bp, synthetic construct
  source 1..241: mol_type = other DNA
ggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggattt

SEQ ID NO: 31 - DNA, 124 bp, synthetic construct
  source 1..124: mol_type = other DNA
aacttttaaaagaaaaggggggattggggggtacagtgcaggggaaagaatagtagacataatagcaacagacatacaaactaaagaattacaaaaacaaattacaaaaattcaaaattttatc

SEQ ID NO: 32 - DNA, 146 bp, synthetic construct
  source 1..146: mol_type = other DNA
tcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaata

SEQ ID NO: 33 - DNA, 468 bp, synthetic construct
  source 1..468: mol_type = other DNA
gggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatc

SEQ ID NO: 34 - DNA, 902 bp, synthetic construct
  source 1..902: mol_type = other DNA
tggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggattt

SEQ ID NO: 35 - DNA, 1510 bp, synthetic construct
  source 1..1510: mol_type = other DNA
tggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatc

SEQ ID NO: 36 - DNA, 5503 bp, synthetic construct
  source 1..5503: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctggctagcgtacgccaccatgctgagggccaagaaccagctgttcctgctgtccccacactacctgcgccaggtgaaggagagctccggctctaggctgatccagcagcgcctgctgcaccagcagcagccactgcaccctgagtgggccgccctggccaagaagcagctgaagggcaagaatcccgaggacctgatctggcacaccccagagggcatcagcatcaagcccctgtattccaagagggacaccatggatctgccagaggagctgccaggagtgaagcctttcacacgcggcccttacccaaccatgtatacatttcggccctggaccatcagacagtacgccggcttcagcacagtggaggagtccaacaagttttataaggacaatatcaaggcaggacagcagggcctgagcgtggcctttgatctggccacacaccgcggctacgactccgataaccccagggtgcgcggcgacgtgggaatggcaggagtggcaatcgacaccgtggaggatacaaagatcctgttcgatggcatccctctggagaagatgtccgtgtctatgaccatgaacggcgccgtgatccccgtgctggccaacttcatcgtgacaggcgaggagcagggcgtgcctaaggagaagctgaccggcacaatccagaacgacatcctgaaggagttcatggtgcggaatacctatatctttccccctgagccatccatgaagatcatcgccgacatcttcgagtacacagccaagcacatgcccaagtttaatagcatctccatctctggctatcacatgcaggaggcaggagcagacgcaatcctggagctggcctacaccctggccgatggcctggagtattcccggaccggcctgcaggcaggcctgacaatcgacgagttcgcccctagactgtctttcttttggggcatcggcatgaacttttacatggagatcgccaagatgagggcaggccggagactgtgggctcacctgatcgagaagatgttccagcctaagaatagcaagtccctgctgctgagagcccactgccagacctccggatggagcctgacagagcaggacccatataacaatatcgtgagaaccgccatcgaggcaatggcagccgtgttcggaggaacccagtctctgcacacaaacagctttgatgaggccctgggcctgcctaccgtgaagagcgcccggatcgccagaaatacacagatcatcatccaggaggagtctggcatcccaaaggtggcagatccatggggaggaagctacatgatggagtgcctgacaaacgacgtgtatgatgccgccctgaagctgatcaatgagatcgaggagatgggaggaatggcaaaggcagtggcagagggaatcccaaagctgcggatcgaggagtgtgcagcaaggcgccaggcaagaatcgactctggcagcgaagtgatcgtgggcgtgaacaagtaccagctggagaaggaggacgccgtggaggtgctggccatcgataacacctccgtgcggaatagacagatcgagaagctgaagaagatcaagtctagccgggatcaggccctggccgagagatgcctggccgccctgacagagtgtgcagcctctggcgacggaaacatcctggccctggccgtggatgccagccgggccagatgtaccgtgggagagatcacagacgccctgaagaaggtgttcggcgagcacaaggccaatgataggatggtgagcggcgcctatcgccaggagttcggcgagtccaaggagatcacctctgccatcaagagggtgcacaagtttatggagagggagggcagacggcccaggctgctggtggcaaagatgggacaggacggacacgataggggagccaaagtgatcgcaaccggcttcgcagacctgggatttgacgtggacatcggccctctgttccagacaccacgggaggtggcacagcaggcagtggacgcagatgtgcacgcagtgggcatcagcaccctggccgcaggacacaagacactggtgcctgagctgatcaaggagctgaactccctgggcagaccagacatcctggtcatgtgcggcggcgtgatcccaccccaggattacgagttcctgtttgaagtgggcgtgtctaacgtgttcggccctggaaccaggatccctaaggcagccgtgcaggtgctggacgacatcgagaagtgtctggagaagaagcagcagagcgtgtgatgagtcgactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctagataatccataaagtaggaaacactacacgattccataaagtaggaaacactacaaccggttccataaagtaggaaacactacatcactccataaagtaggaaacactacacccgggtcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 37 - protein, 750 aa, synthetic construct
MLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 38 - DNA, 2253 bp, synthetic construct
  source 1..2253: mol_type = other DNA
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggtattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgcaagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaatcccaaaggctgccgttcaggtgcttgatgatattgagaagtgtttggaaaagaagcagcaatctgtataa

SEQ ID NO: 39 - DNA, 2253 bp, synthetic construct
  source 1..2253: mol_type = other DNA
atgctgagggccaagaaccagctgttcctgctgtccccacactacctgcgccaggtgaaggagagctccggctctaggctgatccagcagcgcctgctgcaccagcagcagccactgcaccctgagtgggccgccctggccaagaagcagctgaagggcaagaatcccgaggacctgatctggcacaccccagagggcatcagcatcaagcccctgtattccaagagggacaccatggatctgccagaggagctgccaggagtgaagcctttcacacgcggcccttacccaaccatgtatacatttcggccctggaccatcagacagtacgccggcttcagcacagtggaggagtccaacaagttttataaggacaatatcaaggcaggacagcagggcctgagcgtggcctttgatctggccacacaccgcggctacgactccgataaccccagggtgcgcggcgacgtgggaatggcaggagtggcaatcgacaccgtggaggatacaaagatcctgttcgatggcatccctctggagaagatgtccgtgtctatgaccatgaacggcgccgtgatccccgtgctggccaacttcatcgtgacaggcgaggagcagggcgtgcctaaggagaagctgaccggcacaatccagaacgacatcctgaaggagttcatggtgcggaatacctatatctttccccctgagccatccatgaagatcatcgccgacatcttcgagtacacagccaagcacatgcccaagtttaatagcatctccatctctggctatcacatgcaggaggcaggagcagacgcaatcctggagctggcctacaccctggccgatggcctggagtattcccggaccggcctgcaggcaggcctgacaatcgacgagttcgcccctagactgtctttcttttggggcatcggcatgaacttttacatggagatcgccaagatgagggcaggccggagactgtgggctcacctgatcgagaagatgttccagcctaagaatagcaagtccctgctgctgagagcccactgccagacctccggatggagcctgacagagcaggacccatataacaatatcgtgagaaccgccatcgaggcaatggcagccgtgttcggaggaacccagtctctgcacacaaacagctttgatgaggccctgggcctgcctaccgtgaagagcgcccggatcgccagaaatacacagatcatcatccaggaggagtctggcatcccaaaggtggcagatccatggggaggaagctacatgatggagtgcctgacaaacgacgtgtatgatgccgccctgaagctgatcaatgagatcgaggagatgggaggaatggcaaaggcagtggcagagggaatcccaaagctgcggatcgaggagtgtgcagcaaggcgccaggcaagaatcgactctggcagcgaagtgatcgtgggcgtgaacaagtaccagctggagaaggaggacgccgtggaggtgctggccatcgataacacctccgtgcggaatagacagatcgagaagctgaagaagatcaagtctagccgggatcaggccctggccgagagatgcctggccgccctgacagagtgtgcagcctctggcgacggaaacatcctggccctggccgtggatgccagccgggccagatgtaccgtgggagagatcacagacgccctgaagaaggtgttcggcgagcacaaggccaatgataggatggtgagcggcgcctatcgccaggagttcggcgagtccaaggagatcacctctgccatcaagagggtgcacaagtttatggagagggagggcagacggcccaggctgctggtggcaaagatgggacaggacggacacgataggggagccaaagtgatcgcaaccggcttcgcagacctgggatttgacgtggacatcggccctctgttccagacaccacgggaggtggcacagcaggcagtggacgcagatgtgcacgcagtgggcatcagcaccctggccgcaggacacaagacactggtgcctgagctgatcaaggagctgaactccctgggcagaccagacatcctggtcatgtgcggcggcgtgatcccaccccaggattacgagttcctgtttgaagtgggcgtgtctaacgtgttcggccctggaaccaggatccctaaggcagccgtgcaggtgctggacgacatcgagaagtgtctggagaagaagcagcagagcgtgtga

SEQ ID NO: 40 - protein, 418 aa, synthetic construct
MPMLLPHPHQHFLKGLLRAPFRCYHFIFHSSTHLGSGIPCAQPFNSLGLHCTKWMLLSDGLKRKLCVQTTLKDHTEGLSDKEQRFVDKLYTGLIQGQRACLAEAITLVESTHSRKKELAQVLLQKVLLYHREQEQSNKGKPLAFRVGLSGPPGAGKSTFIEYFGKMLTERGHKLSVLAVDPSSCTSGGSLLGDKTRMTELSRDMNAYIRPSPTRGTLGGVTRTTNEAILLCEGAGYDIILIETVGVGQSEFAVADMVDMFVLLLPPAGGDELQGIKRGIIEMADLVAVTKSDGDLIVPARRIQAEYVSALKLLRKRSQVWKPKVIRISARSGEGISEMWDKMKDFQDLMLASGELTAKRRKQQKVWMWNLIQESVLEHFRTHPTVREQIPLLEQKVLIGALSPGLAADFLLKAFKSRD

SEQ ID NO: 41 - DNA, 1257 bp, synthetic construct
  source 1..1257: mol_type = other DNA
atgcccatgctgctaccacatcctcaccagcatttcctaaaaggccttttaagagcacctttccgatgttaccacttcatctttcactcaagtactcatctcggatcaggaatcccatgtgctcagccgtttaattctcttggactccattgtacaaagtggatgctgctgtcagatggcttaaagagaaaattatgtgtacaaacaaccttaaaggaccacacagaaggactttctgataaagagcaaagatttgtggataaactttatactggtttaatccaagggcaaagggcctgtttagcagaggccataactcttgtagaatcaactcacagcaggaaaaaggagttagcccaggtgcttcttcagaaagtattactttaccacagagaacaagaacaatcaaataaaggaaaaccactagcatttcgagtaggattgtctgggccccctggtgctggaaaatcaacatttatagaatattttggaaaaatgcttactgagagagggcacaaattatctgtgctagctgtggacccttcttcttgtactagtggtggatcactcttaggtgataaaacccgaatgactgagttatcaagagatatgaatgcatacatcaggccatctcctactagaggaactttaggaggcgtgacaaggaccacaaatgaagctattctgttgtgtgaaggagcgggatatgacataattcttattgaaaccgttggtgtgggtcagtcggagtttgctgttgctgacatggttgacatgtttgttttactactgccaccagcaggaggagatgagctgcagggtatcaaaaggggtataatcgagatggcagatctggtagctgtaactaaatctgatggagacttgattgtgccagctcgaaggatacaagcggaatatgtgagtgcactgaaattactccgcaaacgttcacaagtctggaaaccaaaggtaattcgtatttctgcccgaagtggagaggggatctctgaaatgtgggataaaatgaaagatttccaggacctaatgcttgccagtggggagctgactgccaaacgacggaagcaacagaaagtttggatgtggaatctcattcaggaaagtgtgttagagcatttcaggacccaccccacagtccgggaacagattccacttctggaacaaaaggttctcattggggccctgtccccaggactagcagcagacttcttgttaaaagcttttaaaagcagagactaa

SEQ ID NO: 42 - protein, 250 aa, synthetic construct
MAVCGLGSRLGLGSRLGLRGCFGAARLLYPRFQSRGPQGVEDGDRPQPSSKTPRIPKIYTKTGDKGFSSTFTGERRPKDDQVFEAVGTTDELSSAIGFALELVTEKGHTFAEELQKIQCTLQDVGSALATPCSSAREAHLKYTTFKAGPILELEQWIDKYTSQLPPLTAFILPSGGKISSALHFCRAVCRRAERRVVPLVQMGETDANVAKFLNRLSDYLFTLARYAAMKEGNQEKIYMKNDPSAESEGL

SEQ ID NO: 43 - DNA, 753 bp, synthetic construct
  source 1..753: mol_type = other DNA
atggctgtgtgcggcctggggagccgtcttggcctggggagccgtcttggcctgcgcgggtgcttcggcgccgccaggctcctgtatccccgtttccagagccgcggccctcagggcgtggaagacggggacaggccacagccttcctcgaagacacccaggatccccaagatttacaccaaaacgggagacaaagggttttctagtaccttcacaggagaaaggagacccaaagatgaccaagtgtttgaagccgtgggaactacagatgaattaagttcagctattgggtttgctctggaattagtcacagaaaagggccatacatttgccgaagagcttcagaaaatccagtgcacattgcaggacgtcggctcggccctggcgacaccatgctcctcggcccgggaggctcacttaaagtataccacgttcaaggcggggcccatcctggagctggagcagtggatcgacaagtacaccagccagctcccaccactcacggccttcatcctgccttcgggaggcaagatcagctcggcgctgcatttctgccgggccgtgtgccgccgggccgagagacgtgtggtgcctcttgtccagatgggagagaccgatgcgaacgtggccaagttcttaaacagactcagtgactatctcttcacgctagccagatatgcagccatgaaggaggggaatcaagagaaaatatacatgaaaaatgacccatcggccgagtctgagggactctga

SEQ ID NO: 44 - protein, 296 aa, synthetic construct
MANVLCNRARLVSYLPGFCSLVKRVVNPKAFSTAGSSGSDESHVAAAPPDICSRTVWPDETMGPFGPQDQRFQLPGNIGFDCHLNGTASQKKSLVHKTLPDVLAEPLSSERHEFVMAQYVNEFQGNDAPVEQEINSAETYFESARVECAIQTCPELLRKDFESLFPEVANGKLMILTVTQKTKNDMTVWSEEVEIEREVLLEKFINGAKEICYALRAEGYWADFIDPSSGLAFFGPYTNNTLFETDERYRHLGFSVDDLGCCKVIRHSLWGTHVVVGSIFTNATPDSHIMKKLSGN

SEQ ID NO: 45 - DNA, 891 bp, synthetic construct
  source 1..891: mol_type = other DNA
atggccaatgtgctttgtaacagagccagactggtttcctatctcccaggattttgctctttagttaaaagggttgtcaatcccaaagccttttcgactgcaggatcatcaggttcggatgagtctcatgtggctgctgcacctccagatatatgctctcgaacagtgtggcctgatgaaactatgggaccctttggacctcaagatcagaggttccagcttcctgggaacataggttttgattgtcacctcaatgggactgcttcacagaagaaaagcctggttcataaaactttgcctgatgttctagcagaacctttatcaagtgaaagacatgagtttgtgatggcacaatatgtgaatgaatttcagggtaatgatgcacctgttgaacaagaaattaacagtgcagaaacttactttgaaagtgccagagtagagtgtgcaatacagacatgtccagaattgctgcgaaaagattttgaatcactgtttccagaagtagctaatggcaaactaatgattctgactgtaacacaaaaaactaagaatgatatgactgtttggagtgaagaagtagaaattgaaagagaagtgctcttagaaaagttcatcaatggtgctaaggaaatttgctatgctcttcgagctgagggttattgggctgactttattgacccatcatctggtttggcattttttggaccatatacaaacaacactctttttgaaactgatgaacgctaccgacatttaggattctctgttgatgaccttggatgctgtaaagtgattcgtcatagtctctggggtacccatgtagttgtagggagtatcttcactaatgcaacaccagacagccatattatgaagaaattaagtggaaattag

SEQ ID NO: 46 - protein, 176 aa, synthetic construct
MARVLKAAAANAVGLFSRLQAPIPTVRASSTSQPLDQVTGSVWNLGRLNHVAIAVPDLEKAAAFYKNILGAQVSEAVPLPEHGVSVVFVNLGNTKMELLHPLGRDSPIAGFLQKNKAGGMHHICIEVDNINAAVMDLKKKKIRSLSEEVKIGAHGKPVIFLHPKDCGGVLVELEQA

SEQ ID NO: 47 - DNA, 531 bp, synthetic construct
  source 1..531: mol_type = other DNA
atggcgcgggtgctgaaggctgcagccgcgaatgccgtagggcttttttccagacttcaagctcccattccaacagtaagagcttcttccacatcacagcccttggatcaagtgacaggttctgtgtggaacctgggtcgactcaaccatgtagccatagcagtgccagatttggaaaaggctgcagcattttataagaatattctgggggcccaggtaagtgaagcggtccctcttcctgaacatggagtatctgttgtttttgtcaacctgggaaataccaagatggaactgcttcatccattgggacgtgacagtccaattgcaggttttctgcagaaaaacaaggctggaggaatgcatcacatctgcatcgaggtggataatattaatgcagctgtgatggatttgaaaaaaaagaagatccgcagtctaagtgaagaggtcaaaataggagcacatggaaaaccagtgatttttctccatcctaaagactgtggtggagtccttgtggaactggagcaagcttga

SEQ ID NO: 48 - DNA, 17 bp, synthetic construct
  source 1..17: mol_type = other DNA
actgacgctctcgcacc

SEQ ID NO: 49 - DNA, 17 bp, synthetic construct
  source 1..17: mol_type = other DNA
tctcgacgcaggactcg

SEQ ID NO: 50 - DNA, 20 bp, synthetic construct
  source 1..20: mol_type = other DNA
atctctctccttctagcctc

SEQ ID NO: 51 - DNA, 18 bp, synthetic construct
  source 1..18: mol_type = other DNA
ggcacacgtggcttttcg

SEQ ID NO: 52 - DNA, 24 bp, synthetic construct
  source 1..24: mol_type = other DNA
ggtgaacctcgtaagtttatgcaa

SEQ ID NO: 53 - DNA, 24 bp, synthetic construct
  source 1..24: mol_type = other DNA
tcaggacgtcgagtggacacggtg

SEQ ID NO: 54 - DNA, 20 bp, synthetic construct
  source 1..20: mol_type = other DNA
ggctgttgggcactgacaat

SEQ ID NO: 55 - DNA, 17 bp, synthetic construct
  source 1..17: mol_type = other DNA
acgtcccgcgcagaatc

SEQ ID NO: 56 - DNA, 23 bp, synthetic construct
  source 1..23: mol_type = other DNA
tttccttggctgctcgcctgtgt

SEQ ID NO: 57 - DNA, 20 bp, synthetic construct
  source 1..20: mol_type = other DNA
accgattccagatgattggc

SEQ ID NO: 58 - DNA, 22 bp, synthetic construct
  source 1..22: mol_type = other DNA
tccatattaatgcagtgcttgc

SEQ ID NO: 59 - DNA, 24 bp, synthetic construct
  source 1..24: mol_type = other DNA
agaggcctgtcctgcagctcatgg